(19) United States
(12) Patent Application Publication
(10) Pub. No.: US 2021/0207108 A1
(43) Pub. Date: Jul. 8, 2021

(54) TWO PHASE SUSTAINABLE PHOTOPRODUCTION VIA CO-CULTIVATION OF ENCAPSULATED, CARBOHYDRATE-PRODUCING CYANOBACTERIA

(71) Applicant: Board of Trustees of Michigan State University, East Lansing, MI (US)

(72) Inventors: Daniel Christoper Ducat, Lansing, MI (US); Taylor Weiss, Gilbert, AZ (US)

(21) Appl. No.: 17/143,045

(22) Filed: Jan. 6, 2021

Related U.S. Application Data
(63) Continuation of application No. 16/250,672, filed on Jan. 17, 2019.
(60) Provisional application No. 62/618,859, filed on Jan. 18, 2018.

(51) Int. Cl. C12N 9/12; B09C 1/10; C12P 39/00

(52) U.S. Cl. CPC C12N 9/1205; C12Y 207/01069; B09C 1/002; C12P 39/00; B09C 2101/00; B09C 1/10

(57) ABSTRACT

Described herein are cyanobacteria encapsulated in hydrogels. Also described herein are consortia that include encapsulated cyanobacteria and heterotrophic microbes. The encapsulated cyanobacteria can be autotrophic and can provide nutrients to the heterotrophic microbes, which can utilize the nutrients to grow and produce useful products.

### CLAIM OF PRIORITY

This application is a continuation of U.S. patent application Ser. No. 16/250,672, filed Jan. 17, 2019, which claims benefit of priority to the filing date of U.S. Provisional Application Ser. No. 62/618,859, filed Jan. 18, 2018, the contents of which are specifically incorporated herein by reference in their entirety.

### GOVERNMENT SUPPORT

This invention was made with government support under 1437657 and 1144152 awarded by National Science Foundation, and under DE-SC0012658 awarded by the U.S. Department of Energy. The government has certain rights in the invention.

### BACKGROUND OF THE INVENTION

From plastics to pharmaceuticals, synthetic organic compounds are predominately manufactured from petrochemical sources. Plastic production alone already consumes 10% of US oil and gas each year, and the market is continuing to grow at 15% a year (Criddle et al., 2014). Use of biodegradable plastic could mitigate the consumption of fossil byproducts and reduce the persistence of waste materials in the environment, yet its production typically requires the microbial conversion of plant-based carbohydrate feedstocks that could otherwise be used as a food source.

Bioplastic production has a significant carbon footprint and is vulnerable to the economics of volatile energy and food markets (Cheali et al., 2016; Ghatak, 2011; Tegtmeier and Duffy, 2004). Genetic engineering of a single species of cyanobacteria or algae may reroute cellular resources towards overproduction of a target renewable product (Leong et al., 2014; Reddy et al., 2013; Venkata Mohan and Venkateswar Reddy, 2013). However, such a monoculture approach to bioproduction requires extensive research and development to optimize each strain and is vulnerable to economic shifts during the development and application of such strains—such as exposure to volatile petroleum markets when creating a biofuel (Cheali et al., 2016). Furthermore, monocultures of algae and cyanobacteria are vulnerable to many inefficiencies when grown in scaled cultures, including contamination by foreign microbial species (Gavrilescu and Chisti, 2005; Vickers et al., 2012).

### SUMMARY

Described herein are cyanobacteria encapsulated in hydrogels. Encapsulation of cyanobacteria can block predation by grazer microbial species, infection by cyanobacterial viruses, and slows division. Such slower cell division can reduce loss of genetically engineered traits and reduce the incidence of genetic mutation. The encapsulated cyanobacteria can be modified to express a sucrose/proton symporter.

Also described herein are consortia that include encapsulated (e.g., autotrophic) cyanobacteria and heterotrophic microbes. The encapsulated cyanobacteria can provide nutrients (e.g., carbon-based nutrients) and the heterotrophic microbes can utilize the nutrients to grow and produce useful products. In some cases, the encapsulated (e.g., autotrophic) cyanobacteria can be modified to express a sugar transporter that they do not naturally express. In addition, the heterotrophic microbes can be modified to produce products that they do not naturally produce.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1A-1G illustrates axenic characterizations of candidate strains and schematically illustrates processes that the strains can engage in. FIG. 1A is a schematic diagram illustrating engineered microbial community designs. Cyanobacteria engineered to secrete sucrose, CscB⁺S. elongatus (green), captures light and carbon dioxide (CO₂) via photosynthesis. Fixed carbon is secreted as sucrose (black arrows) when induced with IPTG in the presence of osmotic pressure (NaCl). This secreted carbon then supports the growth of B. subtilis (blue), S. cerevisiae (purple), or E. coli (orange) with the final goal of production of target compounds from those heterotrophs. Axenic cscB⁺ S. elongatus was grown in a cyanobacteria/bacteria consortia (abbreviated ^{CoB}BG-11) with IPTG (solid line) and without IPTG (dashed line) to induce sucrose secretion. FIG. 1B graphically illustrates cell density of the cyanobacteria/bacteria consortia (abbreviated ^{CoB}BG-11) with IPTG (solid line) and without IPTG (dashed line). FIG. 1C graphically illustrates sucrose levels in culture supernatants of the cyanobacteria/bacteria consortia (abbreviated ^{CoB}BG-11) with IPTG (solid line) and without IPTG (dashed line). Error bars are standard deviation of 8 biological replicates. FIG. 1D illustrates growth (doubling time) of various heterotrophic species in isolation as characterized via growth rate in co-culture buffer supplemented with 2% sucrose. Error is standard deviation of ≥3 replicates. FIG. 1E-1F illustrate axenic cyanobacteria characterization in cyanobacteria/yeast co-cultures (abbreviated ^{COY}BG-11). FIG. 1E graphically illustrates cell growth of cscB⁺ S. elongatus measured in ^{CoY}BG-11 with IPTG (solid lines) or without (dashed lines) IPTG. FIG. 1F graphically illustrates sucrose levels in culture supernatants measured in cultures of ^{CoY}BG-11 with IPTG (solid lines) or without IPTG (dashed lines). Error bars are standard deviation of five biological replicates. FIG. 1G schematically illustrates an alginate-mediated cyanobacteria/bacteria co-culture. S. elongatus CscB, grown phototrophically under osmotic stress and encapsulated within barium-alginate, can excrete fixed carbon via the heterologously-expressed sucrose transporter (CscB), through the hydrogel, and into the medium. Planktonic (i.e., “free floating”) Halomonas boliviensis heterotrophic cells outside of the alginate bead can uptake secreted sucrose for growth or synthesis of polyhydroxybutyrate (PHB), depending on nitrogen availability.

FIG. 2A-2F illustrate that S. elongatus supports growth of microbial communities in batch culture. Batch cultures of cyanobacteria, cscB⁺ S. elongatus (green), in co-culture with B. subtilis (blue), S. cerevisiae (purple), or E. coli (orange) were grown in constant light. The cscB gene is a proon/sucrose symporter that was engineered into S. elongatus to generate a modified cscB⁺ S. elongatus. FIG. 2A shows the numbers of colony forming units of cscB⁺ S. elongatus cells/mL (green, square symbols and top dashed line) determined by flow cytometry every 12 hours for co-cultures containing B. subtilis (blue lines). The dashed line shows growth without induction of cscB expression (in cscB⁺ S. elongatus) while the solid lines show growth with induced cscB expression (in cscB⁺S. elongatus). Note that the B. subtilis exhibits little or no growth when there is no induction of cscB expression in the cscB⁺S. elongatus. FIG. 2B shows the numbers of colony forming units of S. cerevisiae strain W303 (purple, squares) while the green lines show cscB⁺ S. elongalus growth. Solid lines show growth with induced cscB expression (in cscB⁺ S. elongatus) while dashed lines show growth without induced cscB expression (in cscB⁺ S. elongatis). Note that the of S. cerevisiae exhibits little or no increased growth when there is no induction of cscB expression in the cscB⁺S. elongatus. FIG. 2C illustrates axenic heterotroph growth as tested in defined media with varying sucrose concentrations. The range of sucrose that cscB+S. elongatus can secrete in 48 hours is denoted by a green box in FIG. 2C. FIG. 2D shows the numbers of colony forming units of a strain of S. cerevisiae W303^{Clump} (purple; originally called Recreated02 in Koschwanez et al. 2013) which contain mutations in genes CSE2, IRA1, MTH1, and UBR1 that enhance fitness in dilute sucrose in co-culture with cscB⁺ S. elangatus (green). Note that the of S. cerevisiae exhibits better growth compared to the results shown in FIG. 2B even when there is no induction of cscB expression in the cscB⁺ S. elongatus. FIG. 2E shows the numbers of colony forming units of E. cli (orange lines) while the green lines show cscB⁺ S. elongatus growth. Solid lines show growth with induced cscB expression (in cscB⁺S. elongatus) while dashed lines show growth without induced cscB expression (in cscB⁺S. elongatus). FIG. 2F also shows the numbers of colony forming units of E. coli (orange lines) while the green lines show cscB S. elongatus growth. Solid lines show growth with induced cscB expression (in cscB⁺ S. elongatus) while dashed lines show growth without induced cscB expression (in cscB⁺ S. elangatus). Growth in co-cultures with uninduced (dashed lines) or induced cscB expression (solid lines) are shown. Heterotroph viability was monitored by colony forming unit (CFU) formation for all B. subtilis (in FIG. 2A: blue), S. cerevisiae (FIG. 2B S. cerevisiae strain W303, purple; FIG. 2D S. cerevisiae strain W303Clump, purple), and E. coli (FIG. 2E E. coli strain W, orange; FIG. 2F, E. coli strain WΔcscR, orange) co-cultures. Data for FIGS. 2A, 2B, 2D, 2E, and 2F are representative, same-day experiments where error bars are the standard error in three biological replicates.

FIG. 3A-3M illustrate negative cyanobacteria-heterotroph interactions and solutions thereto. FIG. 3A schematically illustrates engineered consortia with un-engineered interactions where the cyanobacteria can have negative effects on heterotroph growth. FIG. 3B graphically illustrates growth of B. subtilis 3610 co-cultured with various concentrations of S. elongatus (dark green backgrounds indicate increasing S. elongatus concentrations). FIG. 3C graphically illustrates growth of S. cerevisiae W303^{Clump} co-cultured with various concentrations of S. elongatus. FIG. 3D graphically illustrates growth of E. coli WΔcscR co-cultured with various concentrations of S. elongatus. For FIGS. 3B-3D, S. elongatus and heterotroph CFU/mL were determined after 12 hours of cultivation in either light or dark. Ratios of CFU in light compared to CFU in dark are reported. FIG. 3E graphically illustrates that additional heterotrophic prokaryotes, including B. subtilis production strain 168, exhibit sensitivity to S. elongatus in the light. FIG. 3F graphically illustrates that additional heterotrophic prokaryotes, including E. coli strain W, exhibit sensitivity to S. elongatus in the light. Co-cultures were set up with S. elongatus of varying concentrations (increasingly dark green backgrounds) and heterotroph CFUs were determined after 12 hours of exposure to either light or dark. Ratios of CFU in light compared to CFU in dark are reported in FIG. 3B-3F. Thick horizontal lines represent the average measurement for each condition while thin horizontal lines represent one standard deviation from the mean. P-values of two-tailed t-tests with Welch's correction are denoted with asterisks: * 0.01 to 0.05, ** 0.001 to 0.01, *** 0.0001 to 0.001, **** <0.0001. FIG. 3G schematically illustrates engineered consortia with un-engineered interactions where the heterotrophs have positive effects on cyanobacteria. FIG. 3H graphically illustrates positive effects of various heterotrophs on cyanobacteria in liquid culture as evidenced by the number of cyanobacteria cells measured in co-cultures relative to axenic controls after 48 hours in constant light. These co-cultures were inoculated with two orders of magnitude fewer cscB+ S. elongatus (about 1.7×10⁶ cells/mL) than the co-cultures depicted in FIG. 2 (about 1.7×10⁸ cells/mL), and 1 mM IPTG was added to all cultures to induce sucrose export. Thick horizontal lines represent the average measurement for each condition while thin horizontal lines represent one standard deviation from the mean. FIG. 3I graphically illustrates the positive effects of additional heterotrophs on cyanobacteria in previous liquid batch experiments. Averages (thick black lines) and standard deviations (thin black lines) were determined from 10 biological replicates for prokaryotes (B. subtilis 3610 in blue, wild type E. coli W, and E. coli W ΔcscR, in orange) and nine biological replicates for S. cerevisiae (purple). Results from all batch cultures described in FIG. 2 were analyzed by normalizing the number of cyanobacteria in co-cultures to the number of cyanobacteria in axenic control cultures at every data point (every 12 hours for 2 days). Of these points, the maximum value for each culture is plotted (each point is a biological replicate). FIG. 3J graphically illustrates the influence of heterotrophs on cyanobacterial growth on solid media as determined by plating a dilute lawn of cscB+ S. elongatus on ^{CoB}BG-1 agar plates. The cyanobacterial lawn was overlaid with ten-fold serial dilutions of the specified heterotroph strain in constant light with or without IPTG. FIG. 3K-3M illustrate that hyper-oxygenation of shared media contributes to the inhibitory effect of S. elongatus on B. subtilis in co-culture. FIG. 3K graphically illustrates growth of B. subtilis 3610 in co-cultures with high-density S. elongatus (6.6×10 cells/mL) inoculated and grown for 12 hours with supplemented sucrose (2%) in light or dark (grey) when the oxygen evolution Photosystem II inhibitor DCMU (3-(3,4-dichlorophenyl)-1,1-dimethylurea), or vehicle control was added to initial cultures. FIG. 3L graphically illustrates growth of B. subtilis 3610 in co-cultures of B. subtilis 3610 and high-density S. elongatus (6.6×10⁸ cells/mL) inoculated and grown for 12 hours with supplemented sucrose (2%) in light or dark (grey) when antioxidant thiosulfate or vehicle control was added to initial cultures. FIG. 3M graphically illustrates growth of B. subtilis 3610 in co-cultures of B. subtilis 3610 and high-density S. elongatus (6.6×10⁸ cells/mL) under different atmospheric conditions. Co-cultures were split into equal volumes into two sealed containers. One container was sealed with atmospheric oxygen levels. The second was sparged with gas (12:10:82 H₂:CO₂:N₂) to deplete oxygen. Viable B. subtilis CFUs were determined after 12 hours in the light. Biological replicates are represented by points on the graph; hollow circles on the x-axis represent replicates in which no colonies grew. Thick horizontal lines represent the average measurement for each condition while thin horizontal lines represent one standard deviation from the mean. Repeat measure one-way ANOVA was used to determine significant deviation between means. The Holm-Sidak multiple comparison test was used to compare all conditions to the light+vehicle control. P-values are denoted with asterisks: * 0.01 to 0.05, ** 0.001 to 0.01, *** 0.0001 to 0.001.

FIG. 4A-4H illustrate that cyanobacteria-heterotroph co-cultures persist through time and perturbation. FIG. 4A graphically illustrates cell density of representative continuous co-cultures of E. coli W ΔcscR (red, lowest line) and cscB+ S. elongatus (green, middle line) over time. The top line shows total culture optical density at 750 nm. FIG. 4B graphically illustrates cell density of representative continuous co-cultures of W303^{Clump} S. cerevisiae (bottom line) and cscB+ S. elongatus (middle line) over time. The top line shows total culture optical density at 750 nm. The cultures shown in FIGS. 4A-4B were cultured in photobioreactors with 1 mM IPTG to induce CscB expression in S. elongatus. E. coli-containing consortia were grown in constant light while S. cerevisiae communities were exposed to 16:8 hour light/dark photo periods (grey spaces represent darkness). Optical density of the entire culture (black points) as well as counts for the individual cell types were tracked (green S. elongatus, orange E. coli W ΔcscR, purple W303Clump S. cerevisiae). FIG. 4C illustrates the numbers of B. subtilis 3610 (blue) and cscB+ S. elongatus (green) in batch cultures following dilution at 24 hours as monitored over time by viable colony counts and flow cytometry for heterotrophs and cyanobacteria, respectively. FIG. 4D illustrates the numbers of E. coli W ΔcscR (orange) and cscB+ S. elongatus (green) in batch cultures following dilution at 24 hours as monitored by viable colony counts and flow cytometry for heterotrophs and cyanobacteria, respectively. FIG. 4E illustrates an example process for co-culturing a cyanobacteria and a heterotroph. Liquid co-cultures of cscB⁺ S. elongatus with either B. subtilis strain 3610 or E. coli W ΔcscR were inoculated as described for FIG. 2 and grown for 24 hours, then plated on solid ^{CoB}BG-11 agar. Plates were incubated in constant light until visible growth was apparent, whereupon green colonies were picked into individual wells containing liquid media and returned to the incubator. After 3-5 days of growth, cultures were visibly scored for the presence of cyanobacteria (green coloration) and plated on rich media to determine the presence or absence of heterotrophs (e.g., lower left panel: “X” mark wells containing cyanobacteria where heterotrophs were lost). FIG. 4F graphically illustrates that some species persist through co-culture perturbation. The percentages of the wells containing one or both prokaryote(s) from 387 wells and 442 wells are shown. FIG. 4G-4H graphically illustrate extended continuous S. cerevisiae/cscB⁺ S. elongatus co-cultures. For FIG. 4G, cultures of S. elongatus and S. cerevisiae W303^{Clump} were inoculated in photobioreactors under constant light with 1 mM IPTG to induce sucrose export. The number of cyanobacterial and yeast cells within the co-culture were monitored daily by withdrawing samples from the reactor. W303^{Clump} viable cell counts were determined as for other cultures by plating serial dilutions on YEPD media (light purple, lower line). Cell counts for cyanobacteria (top green line) were estimated by measuring OD₇₅₀ of the total culture following a light centrifugation step (100 xg for 30 see) which pelleted >90% of the W303^{Clump} cells but which left smaller cyanobacterial cells in suspension: OD₇₅₀ measurements were compared to a standard curve to estimate cyanobacterial cell number (analysis of cyanobacterial cell density in this experiment is distinct from other, later co-culture data collection—in which FACS analysis was utilized). FIG. 4H shows the standard curve to estimate cyanobacterial cell number for the results shown in FIG. 4G.

FIG. 5A-5D illustrate photoproduction of enzymes and metabolites from co-cultures. FIG. 5A schematically illustrates flexible functionalization of co-cultures accomplished via the addition of heterotrophs capable of producing target compounds. FIG. 5B graphically illustrates natural production and secretion of alpha-amylase by B. subtilis strain 168. Supernatants from 24-hour cultures of B. subtilis 168 alone or in co-culture with cscB+ S. elongatus were as tested for enzymatic activity with (right data points) and without IPTG (middle data points) to induce sucrose secretion. FIG. 5C shows western blots demonstrating the presence of alpha-amylase in co-cultures containing IPTG to induce sucrose secretion by cscB+ S. elongatus. FIG. 5D graphically illustrates that E. coli (orange cells) can produce significant amounts of polyhydroxybutyrate (PHB) when carrying the pAET41 plasmid and co-cultured with cscB+ S. elongatus (green cells). Batch co-cultures of E. coli (with or without pAET41 to enable PHB production) and cscB+ S. elongatus were cultivated for one week with or without IPTG to induce sugar production. Filled circles represent measured values; hollow circles placed on the x-axis represent cultures in which no PHB was formed or was produced at levels below the detection limit. Thick horizontal lines represent the average measurement for each condition while thin horizontal lines represent one standard deviation from the mean.

FIG. 6A-6E graphically illustrates growth of Halomonas boliviensis. FIG. 6A graphically illustrates growth of H. boliviensis in BG11 and M1 media with 140 mM NaCl and variable sucrose (0.05, 0.1, or 0.5% sucrose; w/v) added. FIG. 6B graphically illustrates growth of H. boliviensis in BG11 and M1 media with 0.5% sucrose (w/v) and variable salt (80, 100, 120, or 140 mM NaCl) added (n=3; SEM). FIG. 6C illustrates characteristics of sucrose-fed, rapidly growing H. boliviensis in BG11 media. As shown the H. boliviensis in BG11 media create heterogeneous clumps or flocs, which complicate optical density measurements. FIG. 6D shows images of culture flasks after H. boliviensis was grown for 72 h either in BG11 medium or in M1 medium with sucrose added (0.1, 0.5, 1.0, or 1.5%). While H. boliviensis cells appear to remain suspended in M1 media, cells persistently adhere to the flask surface (as indicated by arrowheads) in BG11 media, complicating quantification by optical density. FIG. 6E shows H. boliviensis grown in BG11 medium with 0.5, 1.0, or 1.5% sucrose, as visualized by light microscopy. As illustrated, increasing sucrose nutrition can increase cell aggregation.

FIG. 7A-7C illustrate S. elongatus CscB planktonic (i.e., “free floating”) cultures. FIG. 7A graphically illustrates S. elongatus CscB planktonic culture as observed by cell density in BG11 or M1 media having different levels of NaCl (80, 100, 120, or 140 mM NaCl) added. FIG. 7B graphically illustrates S. elongatus CscB planktonic culture as observed by sucrose accumulation in BG11 or M1 media having different levels of NaCl (80, 100, 120, or 140 mM NaCl), where the lines are as defined in FIG. 7A. FIG. 7C graphically illustrates S. elongatus CscB planktonic culture as observed by sucrose accumulation per cell density, in BG11 or M1 media with variable salt (80, 100, 120, or 140 mM NaCl) added (n=3; SEM), where the lines are as defined in FIG. 7A.

FIGS. 8A-8K illustrate characteristics of S. elongatus CscB planktonic compared to alginate-encapsulated culture. FIG. 8A illustrates S. elongatus CscB planktonic (solid lines) and encapsulated S. elongatus CscB (dashed lines) cell density after 66 hours incubation in M1 medium with salt (140 mM NaCl (red lines) or 160 mM NaCl (blue lines)) added (n=8; SEM). For the encapsulated condition in FIG. 8A, the OD₇₅₀ is calculated as the sum of the free media measurement plus the OD₇₅₀ relative density that was initially encapsulated. Encapsulated cells do not show significant growth inside beads within the first 72 h (data not shown). FIG. 8B illustrates S. elongatus CscB planktonic (solid lines) and encapsulated S. elongatus CscB (dashed lines) sucrose accumulation after 66 hours incubation in M1 medium with salt (140 mM NaCl (red lines) or 160 mM NaCl (blue lines)) added (n=8; SEM). FIG. 8C graphically illustrates sucrose accumulation per Chla (w/w) after 66 hours of incubation in M1 medium with (140 mM NaCl (red bars) or 160 mM NaCl (blue bars)) added (n=8; SEM). FIG. 8D depicts barium-alginate encapsulated S. elongatus CscB beads. These beads can be suspended in medium containing planktonic H. boliviensis, or other heterotrops for co-culture; inset: ˜2.6 mm beads (10) lined-up, for scale. FIG. 8D shows barium-alginate encapsulated S. elongatus CscB, suspended in medium containing planktonic H. boliviensis, as for a typical co-culture; inset: ˜2.6 mm beads (10) lined-up, for scale. FIG. 8E graphically illustrates Chla concentration of encapsulated and planktonic S. elongatus CscB cultured in M1 medium with 140 mM NaCl (red bars) or 160 mM NaCl (blue bars) after 66 hours of culture. FIG. 8F graphically illustrates planktonic Chla concentration per OD₇₅₀ (n=8; SEM) of encapsulated and planktonic S. elongatus CscB cultured in M1 medium with 140 mM NaCl (red bars) or 160 mM NaCl (blue bars) after 66 hours of culture. FIG. 8G graphically illustrates estimates of sucrose specific productivity of planktonic and encapsulated S. elongatus CscB on a per-cell basis in media containing 140 mM NaCl (red lines) or 160 mM NaCl (blue lines). To estimate the per cell sucrose productivity of S. elongatus CscB between planktonic and encapsulated cultures, sucrose production was evaluated as a function of OD₇₅₀ over time. For planktonic cultures, this was a straightforward comparison of total sucrose secreted per OD₇₅₀. For encapsulated cultures, the quantity of S. elongatus CscB that were originally embedded in Ba-alginate was added to the OD₇₅₀ measurement at each time point sampled (see FIG. 8A-8B). These estimations are based on the assumption that all encapsulated cells remain viable and that no significant cell division occurs within the alginate beads within the first 66 hours after curing. FIG. 8H shows transmission electron micrograph (TEM) of co-cultured, barium-alginate encapsulated S. elongatus CscB beads at day 0 of culture. Scale bar=1 m. FIG. 8I shows transmission electron micrograph (TEM) of co-cultured, barium-alginate encapsulated S. elongatus CscB at day 7 of culture. Scale bar=1 m. FIG. 8J shows transmission electron micrograph (TEM) of co-cultured, barium-alginate encapsulated S. elongatus CscB at day 42 of culture. Scale bar=1 m. FIG. 8K shows transmission electron micrograph (TEM) of co-cultured, barium-alginate encapsulated S. elongatus CscB at day 151 of culture. Scale bar=1 μm.

FIG. 9A-9E graphically illustrates growth and production of PHB and biomass of co-cultures of planktonic H. boliviensis and encapsulated S. elongatus CscB. FIG. 9A graphically illustrates colony forming units (CFU) of planktonic H. boliviensis co-cultured with encapsulated S. elongatus CscB in M1 medium with 140 mM (orange lines) or 160 mM NaCl (blue lines) (n=16; SEM). The CFU of H. boliviensis was measured on 1097 medium. FIG. 9B graphically illustrates growth of the cultures in medium with 140 mM (orange lines) or 160 mM NaCl (blue lines) as measured by OD₆₀₀ measurements. Media was harvested on Day 7 (arrowhead) and Day 14. FIG. 9C graphically illustrates PHB concentration (mg/L) of media on days 7 and 14 of H. boliviensis and encapsulated S. elongatus CscB co-cultures in M1 medium with 140 mM NaCl (orange bars) or 160 mM NaCl (blue bars). FIG. 9D graphically illustrates dry biomass on days 7 and 14 of H. boliviensis and encapsulated S. elongatus CscB co-cultures in M1 medium with 140 mM NaCl (orange bars) or 160 mM NaCl (blue bars) (n=16; SEM). FIG. 9E graphically illustrates titers of H. boliviensis over time in medium with 140 mM NaCl (orange bars) or 160 mM NaCl (blue bars). Measurements of soluble sucrose in co-cultures of S. elongatus CscB and H. boliviensis in the supernatant of the 2-week co-culture shown in FIG. 9A, as determined by the Sucrose/D-Glucose Assay Kit (lines). The amounts of sucrose are plotted as lines for cultures in medium with 140 mM NaCl (orange lines) or 160 mM NaCl (blue lines).

FIG. 10A-10G illustrate features of co-cultures containing planktonic H. boliviensis and encapsulated S. elongatus CscB in M2 medium (nitrogen-limited media) with 170 mM NaCl. Spent medium was periodically removed, for biomass harvesting, and replaced with fresh medium every 3 or 4 days, leaving the encapsulated S. elongatus CscB beads in place. FIG. 10A graphically illustrates H. boliviensis (top orange line) and heterotrophic contamination (bottom blue line) as measured by colony forming units after plating. FIG. 10B graphically illustrates planktonic co-culture cell density as measured by media optical density (n=12; SEM). Dashed lines at Day 133 in both FIGS. 10A-10B are representative of a back-dilution that occurred, but for which incomplete data was recorded. FIG. 10C graphically illustrates H. boliviensis growth rate in the first 24 h after back-dilution as estimated using both optical density and plating during back dilutions on Days 28-49 (n=72; SEM). FIG. 10D shows light microscopy images of centrifuged, Nile red stained H. boliviensis cells from co-cultures as viewed by brightfield and fluorescence. FIG. 10E shows TEM images of H. boliviensis cells that most frequently contain a single, large, ovoid PHB granule visible in both transverse and longitudinal sections. FIG. 10F graphically illustrates PHB amounts (PHB/dry weight % yield) and dry weight biomass in co-cultured medium harvested during back-dilutions across various time points. FIG. 10G graphically illustrates the average PHB and dry weight productivities at Days 25-161 (n=40; SEM).

FIG. 11A-11B illustrate the instability within S. elongatus/H. boliviensis co-cultures when cyanobacteria are grown planktonically (i.e., as a free-floating culture). Co-cultures of S. elongatus CscB and H. boliviensis were set up as described for FIG. 10, except that cyanobacterial cells were suspended in liquid media rather than encapsulated within barium-alginate. FIG. 11A graphically illustrate the numbers of H. boliviensis colony forming units over time. Viable H. boliviensis cells were monitored by observing colony forming units on solid agar plates (1097 medium). A single back-dilution, whereby 75% of the culture was removed and replaced with fresh M2 101 media, occurred after 72 h (blue arrowhead), analogous to back-dilutions routinely performed upon co-102 cultures with alginate-encapsulated S. elongatus CscB (FIG. 10). FIG. 11B graphically illustrates total chlorophyll a within the mixed co-culture as monitored by extraction and quantification, illustrating the visible “bleaching” of cyanobacterial cells caused by prolonged exposure to low nitrogen conditions. Error bars represent standard deviations from the average of 12 independent cultures; 4 biological replicates with 3 technical replicates each.

FIG. 12A-12B graphically illustrates polyhydroxybutyrate (PHB) production from co-cultures of engineered E. coli with alginate-encapsulated S. elongatus CscB. FIG. 12A graphically illustrates colony counts of E. coli plated on LB agar to determine viable heterotrophic cells in co-culture. At the end of Days 3 and 7, all liquid volume was removed from each flask and fresh M2 media was added back to the remaining barium-alginate beads containing S. elongatus CscB (blue arrowheads). Error bars represent standard deviation between 7 independent cultures (biological replicates). FIG. 12B graphically illustrates the total dry cell biomass collected from the liquid media of each collection (blue arrowheads in FIG. 12A) as weighed and analyzed for PHB content. The total productivity over the course of the 7-day co-culture was used to determine a specific daily productivity, indicating 12.8% of the E. coli biomass was 123 attributable to PHB. Co-cultures were prepared as described for FIG. 10 except that ΔcscR E. coli W cells transformed with pAET41 (to express phbABC) were cultivated with alginate encapsulated S. elongatus CscB.

FIG. 13A-13K illustrates that encapsulated S. elongatus CscB+ cells are more tolerant of the toxin dinitrotoluene (DNT) than non-encapsulated S. elongatus CscB+ cells and that co-cultures of these two species can degrade DNT (e.g., 2,4-DNT). FIG. 13A graphically illustrates the growth of non-encapsulated S. elongatus CscB+ cells at different levels of 2,4-DNT when grown in suspension as measured by absorption (OD₇₅₀). As illustrated, low concentrations of DNT slow the growth rate of the S. elongatus CscB+ cells. FIG. 13B graphically illustrates the viability of non-encapsulated S. elongatus CscB+ cells at different levels of DNT when grown in suspension as measured by extracted chlorophyll a concentrations (Chla μg/ml). As illustrated, concentrations of DNT greater than 0.03 mM led to chlorosis of S. elongatus CscB+ cells, as measured by extracted chlorophyll a. FIG. 13C illustrates encapsulation of S. elongatus CscB+ cells in alginate hydrogel beads. S. elongatus CscB+ cells are viable inside these hydrogels for long time periods (e.g., more than 5 months, see, e.g., FIG. 10). FIG. 13D graphically illustrates that Ba-alginate encapsulated S. elongatus CscB+ cells are resistant to much higher concentrations of 2,4-DNT (at least 0.25 mM) than non-encapsulated S. elongatus CscB+ cells (about 0.03 mM), as evaluated by chlorophyll extraction from beads after 7 days of 2,4-DNT exposure (compare to FIG. 13B). FIG. 13E graphically illustrates that encapsulated S. elongatus CscB+ cells continue to export sucrose at comparable rates to control encapsulated S. elongatus CscB+ cells that are not exposed to the toxin (measured over 24 hours). FIG. 13F illustrates an example of a dnt expression cassette, referred to as pSEVA221-cscRABY. FIG. 13G illustrates the enzymes and products in the Dnt-mediated dinitrotoluene breakdown pathway. FIG. 13H graphically illustrates degradation of 2,4-DNT by engineered P. putida strains bearing the heterologous pathway (P. putida-EM-DNT-S) shown in FIGS. 13F and 13G. The appearance of pathway intermediates 4-methyl-5-dinitrocatechol (4M5NC) and 2-hydroxy-5-methylquinone (2H5MQ) were tracked by their characteristic absorption spectra (absorption maxima at 420 nm and 485 nm, respectively). FIG. 13I graphically illustrates that the degradation pathway intermediate 4M5NC is only produced by strains bearing the heterologous 2,4-DNT processing pathway, as assayed by liquid chromatography/mass spectrometry. FIG. 13J graphically illustrates that P. putida strains can also produce the bioplastic polymer polyhydroxyalkanoate (PHA). The P. putida-EM-DNT-S can synthesize PHA while also degrading 2,4-DNT. FIG. 13K schematically illustrates degradation of dinitrotoluene and production of polyhydroxyalkanoate.

FIG. 14 illustrates that E. coli strains engineered to overexpress the tryptophanase gene (tnaA; yellow cells) can grow in co-culture with sucrose secreting S. elongatus (green cells) and secrete significant quantities of the product indole.

### DETAILED DESCRIPTION

Described herein are mixtures (e.g., co-cultures) of cyanobacteria that can supply nutrients (e.g., sugar) with at least one heterotrophic microorganism modified to produce useful products, and methods for making and using such mixtures. The cyanobacteria can therefore supply the heterotrophic microorganism(s) with nutrients to support their growth and production of useful products. Also as described herein, encapsulation of the cyanobacteria improves the stability of the cyanobacteria and the productivity of the co-cultures.

### Cyanobacteria

Cyanobacteria, also known as blue-green algae, blue-green bacteria, or Cyanophyta, is a phylum of bacteria that obtain their energy through photosynthesis. Cyanobacteria can produce metabolites, such as carbohydrates, proteins, lipids and nucleic acids, from CO₂, water, inorganic salts and light. Any cyanobacteria may be used according to the present disclosure.

Cyanobacteria include both unicellular and colonial species. Colonies may form filaments, sheets or even hollow balls. Some filamentous colonies have the ability to differentiate into several different cell types, such as vegetative cells, the normal, photosynthetic cells that are formed under favorable growing conditions; akinetes, the climate-resistant spores that may form when environmental conditions become harsh; and thick-walled heterocysts, which contain the enzyme nitrogenase, vital for nitrogen fixation.

Heterocysts may also form under the appropriate environmental conditions (e.g., anoxic) whenever nitrogen is necessary. Heterocyst-forming species are specialized for nitrogen fixation and are able to fix nitrogen gas, which cannot be used by plants, into ammonia (NH₃), nitrites (NO₂⁻), or nitrates (NO₃⁻), which can be absorbed by plants and converted to protein and nucleic acids.

Many cyanobacteria also form motile filaments, called hormogonia, which travel away from the main biomass to bud and form new colonies elsewhere. The cells in a hormogonium are often thinner than in the vegetative state, and the cells on either end of the motile chain may be tapered. In order to break away from the parent colony, a hormogonium often must tear apart a weaker cell in a filament, called a neridium.

Each individual cyanobacterial cell typically has a thick, gelatinous cell wall. Cyanobacteria differ from other gram-negative bacteria in that the quorum sensing molecules autoinducer-2 and acyl-homoserine lactones are absent. They lack flagella, but hormogonia and some unicellular species may move about by gliding along surfaces. In water columns, some cyanobacteria float by forming gas vesicles, like in archaea.

Cyanobacteria have an elaborate and highly organized system of internal membranes that function in photosynthesis. Photosynthesis in cyanobacteria generally uses water as an electron donor and produces oxygen as a by-product, though some cyanobacteria may also use hydrogen sulfide, similar to other photosynthetic bacteria. Carbon dioxide is reduced to form carbohydrates via the Calvin cycle. In most forms, the photosynthetic machinery is embedded into folds of the cell membrane, called thylakoids.

In some cases, the amount of oxygen produced by the cyanobacteria can inhibit the growth or functioning of heterotrophic organism that is co-cultured with the cyanobacteria. As illustrated herein, the atmosphere or the culture media in which a consortium of cyanobacteria and heterotrophic cells is cultured can be modulated to reduce the inhibitory effects of such oxygen production. For example, the consortium can be sparged with low-oxygen containing gases or anti-oxidants can be added to the culture medium.

In some cases, the cultures can be sparged with a gas having less than 20% oxygen, or less than 17% oxygen, or less than 15% oxygen, or less than 12% oxygen, or less than 10% oxygen, or less than 7% oxygen, or less than 5% oxygen, or less than 3% oxygen, or less than 1% oxygen. In some cases, the cultures can be sparged with a gas that is devoid of oxygen (e.g., 12:10:82 H₂:CO₂:N₂).

In some cases, antioxidants such as DCMU (3-(3,4-dichlorophenyl)-1,1-dimethylurea), thiosulfate, or a combination thereof can be used in the culture medium. Concentrations of such antioxidants can vary from about 1 μM to 1000 mM, or about 10 μM to 200 mM.

Due to their ability to fix nitrogen in aerobic conditions, cyanobacteria are often found as symbionts with a number of other groups of microorganisms such as fungi (e.g., lichens), corals, pteridophytes (e.g., Azolla), and angiosperms (e.g., Gunnera), among others.

Cyanobacteria are the only group of microorganisms that are able to reduce nitrogen and carbon in aerobic conditions. The water-oxidizing photosynthesis is accomplished by coupling the activity of photosystem (PS) II and I (Z-scheme). In anaerobic conditions, cyanobacteria are also able to use only PS I (i.e., cyclic photophosphorylation) with electron donors other than water (e.g., hydrogen sulfide, thiosulphate, or molecular hydrogen), similar to purple photosynthetic bacteria. Furthermore, cyanobacteria share an archaeal property; the ability to reduce elemental sulfur by anaerobic respiration in the dark. The cyanobacterial photosynthetic electron transport system shares the same compartment as the components of respiratory electron transport. Typically, the plasma membrane contains only components of the respiratory chain, while the thylakoid membrane hosts both respiratory and photosynthetic electron transport.

Cyanobacteria of the present disclosure may be from any genera or species of cyanobacteria that is genetically manipulable, i.e., permissible to the introduction and expression of exogenous genetic material. Examples of cyanobacteria that can be employed include, but are not limited to, the genus Synechocystis, Synechococcus, Thermosynechococcus, Nostoc, Prochlorococcu, Microcystis, Anabaena, Spirulina, and Gloeobacter. In some cases, the cyanobacteria are Synechococcus elongatus. For example, the cyanobacteria can include Synechococcus elongatus PCC 7942.

In some cases, the cyanobacteria can be engineered to express a sucrose transporter. One example of such a sucrose transporter is the CscB transporter protein. An example of a CscB protein that is useful for sucrose export is an Escherichia coli H⁺/sucrose symporter CscB, for example having the following sequence (SEQ ID NO:1).

| | |
|---|---|
| 1 | MALNIPFRNA YYRFASSYSF LFFISWSLWW SLYAIWLKGH |
| 41 | LGLTGTELGT LYSVNQFTSI LFMMFYGIVQ DKLGLKKPLI |
| 81 | WCMSFILVLT GPFMIYVYEP LLQSNFSVGL ILGALFFGLG |
| 121 | YLAGCGLLDS FTEKMARNFH FEYGTARAWG SFGYAIGAFF |
| 161 | AGIFFSISPH INFWLVSLFG AVFMMINMRF KDKGHQCVAA |
| 201 | DAGGVKKEDF IAVFKDRNFW VFVIFIVGTW SFYNIFDQQL |
| 241 | FPVFYAGLFE SHDVGTRLYG YLNSFQVVLE ALCMAIIPFF |
| 281 | VNRVGPKNAL LIGVVIMALR ILSCALFVNP WIISLVKLLH |
| 321 | AIEVPLCVIS VFKYSVANFD KRLSSTIFLI GFQIASSLGI |
| 361 | VLLSTPTGIL FDHAGYQTVF FAISGIVCLM LLFGIFFLSK |
| 401 | KREQIVMETP VPSAI |

A nucleotide sequence that encodes the SEQ ID NO:1 Escherichia coli CscB protein is shown below as SEQ ID NO:2.

| | |
|---|---|
| 1 | ATGGCACTGA ATATTCCATT CAGAAATGCG TACTATCGTT |
| 41 | TTGCATCCAG TTACTCATTT CTCTTTTTTA TTTCCTGGTC |
| 81 | GCTGTGGTGG TCGTTATACG CTATTTGGCT GAAAGGACAT |
| 121 | CTAGGATTAA CAGGGACGGA ATTAGGTACA CTTTATTCGG |
| 161 | TCAACCAGTT TACCAGCATT CTATTTATGA TGTTCTACGG |
| 201 | CATCGTTCAG GATAAACTCG GTCTGAAGAA ACCGCTCATC |
| 241 | TGGTGTATGA GTTTCATTCT GGTCTTGACC GGACCGTTTA |
| 281 | TGATTTACGT TTATGAACCG TTACTGCAAA GCAATTTTTC |
| 321 | TGTAGGTCTA ATTCTGGGGG CGCTCTTTTT TGGCCTGGGG |
| 361 | TATCTGGCGG GATGTGGTTT GCTTGACAGC TTCACTGAAA |
| 401 | AAATGGCGCG AAATTTTCAT TTCGAATATG GAACAGCGCG |
| 441 | CGCCTGGGGA TCTTTTGGCT ATGCTATTGG CGCGTTCTTT |
| 481 | GCCGGCATAT TTTTTAGTAT CAGTCCCCAT ATCAACTTCT |
| 521 | GGCTGGTCTC GCTATTTGGC GCTGTATTTA TGATGATCAA |
| 561 | CATGCGTTTT AAAGATAAGG GTCACCAGTG TGTAGCGGCG |
| 601 | GATGCGGGAG GGGTAAAAAA AGAGGATTTT ATCGCAGTTT |
| 641 | TCAAGGATCG AAACTTCTGG GTTTTTGTCA TATTTATTGT |
| 681 | GGGGACGTGG TCTTTCTATA ACATTTTTGA TCAACAACTC |
| 721 | TTTCCTGTCT TTTATGCAGG TTTATTCGAA TCACACGATG |
| 761 | TAGGAACGCG CCTGTATGGT TATCTCAACT CATTCCAGGT |
| 801 | GGTACTCGAA GCGCTGTGCA TGGCGATTAT TCCTTTCTTT |
| 841 | GTGAATCGGG TAGGGCCAAA AAATGCATTA CTTATCGGTG |
| 861 | TTGTGATTAT GGCGTTGCGT ATCCTTTCCT GCGCGTTGTT |
| 921 | CGTTAACCCC TGGATTATTT CATTAGTGAA GCTGTTACAT |
| 961 | GCCATTGAGG TTCCACTTTG TGTCATATCC GTCTTCAAAT |
| 1001 | ACAGCGTGGC AAACTTTGAT AAGCGCCTGT CGTCGACGAT |
| 1041 | CTTTCTGATT GGTTTTCAAA TTGCCAGTTC GCTTGGGATT |
| 1081 | GTGCTGCTTT CAACGCCGAC TGGGATACTC TTTGACCACG |
| 1121 | CAGGCTACCA GACAGTTTTC TTCGCAATTT CGGGTATTGT |
| 1161 | CTGCCTGATG TTGCTATTTG GCATTTTCTT CCTGAGTAAA |
| 1201 | AAACGCGAGC AAATAGTTAT GGAAACGCCT GTACCTTCAG |
| 1241 | CAATATAG |

The NCBI database provides a variety of sucrose transporters (also called sucrose symporter or sucrose permeases). For example, an Escherichia coli CscB sucrose permease that has accession number CAA45274 can be employed and has the following sequence (SEQ ID NO:3).

| | |
|---|---|
| 1 | MALNIPFRNA YYRFASSYSF LFFISWSLWW SLYAIWLKGH |
| 41 | LGLTGTELGT LYSVNQFTSI LFMMFYGIVQ DKLGLKKPLI |
| 81 | WCMSFILVLT GPFMIYVYEP LLQSNFSVGL ILGALFFGLG |
| 121 | YLAGCGLLDS FTEKMARNFH FEYGTARAWG SFGYAIGAFF |
| 161 | AGIFFSISPH INFWLVSLFG AVFMMINMRF KDKDHQCIAA |
| 201 | DAGGVKKEDF IAVFKDRNFW VFVIFIVGTW SFYNIFDQQL |
| 241 | FPVFYAGLFE SHDVGTRLYG YLNSFQVVLE ALCMAIIPFF |
| 281 | VNRVGPKNAL LIGVVIMALR ILSCALFVNP WIISLVKLLH |
| 321 | AIEVPLCVIS VFKYSVANFD KRLSSTIFLI GFQIASSLGI |
| 361 | VLLSTPTGIL FDHAGYQTVF FAISGIVCLM LLFGIFFLSK |
| 401 | KREQIVMETP VPSAI |

| | |
|---|---|
| 1 | GTCGACAAGA ACTGGAAAGT GAGAAGTAAA CGGCGAGGCC |
| 41 | GCTCTTATCT CTAAATAGGA CATGAATTTT TTAACGACAG |
| 81 | GCAGGTAATT ATGGCACTGA ATATTCCATT CAGAAATGCG |
| 121 | TACTATCGTT TTGCATCCAG TTACTCATTT CTCTTTTTTA |
| 161 | TTTCCTGGTC GCTGTGGTGG TCGTTATACG CTATTTGGCT |
| 201 | GAAAGGACAT CTAGGATTAA CAGGGACGGA ATTAGGTACA |
| 241 | CTTTATTCGG TCAACCAGTT TACCAGCATT CTATTTATGA |
| 281 | TGTTCTACGG CATCGTTCAG GATAAACTCG GTCTGAAGAA |
| 321 | ACCGCTCATC TGGTGTATGA GTTTCATTCT GGTCTTGACC |
| 361 | GGACCGTTTA TGATTTACGT TTATGAACCG TTACTGCAAA |
| 401 | GCAATTTTTC TGTAGGTCTA ATTCTGGGGG CGCTCTTTTT |
| 441 | TGGCCTGGGG TATCTGGCGG GATGCGGTTT GCTTGACAGC |
| 481 | TTCACCGAAA AAATGGCGCG AAATTTTCAT TTCGAATATG |
| 521 | GAACAGCGCG CGCCTGGGGA TCTTTTGGCT ATGCTATTGG |
| 561 | CGCGTTCTTT GCCGGTATAT TTTTTAGTAT CAGTCCCCAT |
| 601 | ATCAACTTCT GGTTGGTCTC GCTATTTGGC GCTGTATTTA |
| 641 | TGATGATCAA CATGCGTTTT AAAGATAAGG ATCACCAGTG |
| 681 | CATAGCGGCG GATGCGGGAG GGGTAAAAAA AGAGGATTTT |
| 721 | ATCGCAGTTT TCAAGGATCG AAACTTCTGG GTTTTCGTCA |
| 761 | TATTTATTGT GGGGACGTGG TCTTTCTATA ACATTTTTGA |
| 801 | TCAACAACTC TTTCCTGTCT TTTATGCAGG TTTATTCGAA |
| 841 | TCACACGATG TAGGAACGCG CCTGTATGGT TATCTCAACT |
| 861 | CATTCCAGGT GGTACTCGAA GCGCTGTGCA TGGCGATTAT |
| 921 | TCCTTTCTTT GTGAATCGGG TAGGGCCAAA AAATGCATTA |
| 961 | CTTATCGGTG TTGTGATTAT GGCGTTGCGT ATCCTTTCCT |
| 1001 | GCGCGTTGTT CGTTAACCCC TGGATTATTT CATTAGTGAA |
| 1041 | GCTGTTACAT GCCATTGAGG TTCCACTTTG TGTCATATCC |
| 1081 | GTCTTCAAAT ACAGCGTGGC AAACTTTGAT AAGCGCCTGT |
| 1121 | CGTCGACGAT CTTTCTGATT GGTTTTCAAA TTGCCAGTTC |
| 1161 | GCTTGGGATT GTGCTGCTTT CAACGCCGAC TGGGATACTC |
| 1201 | TTTGACCACG CAGGCTACCA GACAGTTTTC TTCGCAATTT |
| 1241 | CGGGTATTGT CTGCCTGATG TTGCTATTTG GCATTTTCTT |
| 1281 | CCTGAGTAAA AAACGCGAGC AAATAGTTAT GGAAACGCCT |
| 1321 | GTACCTTCAG CAATATAGAC GTAAACTTTT TCCGGTTGTT |
| 1361 | GTCGATATC |

Another sucrose transporter can be used such as the sucrose transport protein from uncultured bacterium that has accession number CCG34807.1, provided below as SEQ ID NO:5.

| | |
|---|---|
| 1 | MIMASATKSA WKNPSYLQSS FGIFMFFCSW GIWWSFFQRW |
| 41 | LISGVGLTNA EVGTIYSINS LATLVIMFVY GVIQDQLGIK |
| 81 | RKLVIVVSVI AACVGPFVQF VYAPMILAGG TTRWIGALIG |
| 121 | SIVLSAGFMS GCSLFEAVTE RYSRKFGFEY GQSRAWGSFG |
| 161 | YAIVALCAGF LFNINPLINF WVGSAFGLGM LLVYAFWVPA |
| 201 | EQKEELKKET DPNAAPTNPS LKEMVAVLKM PTLWVLIVFM |
| 241 | LLTNTFYTVF DQQMFPTYYA NLFPTEEIGN ATYGTLNGFQ |
| 281 | VFLESAMMGV VPIIMKKIGV RNALLLGATV MFLRIGLCGV |
| 321 | FHDPITISIV KLFHSIEVPL FCLPAFRYFT LHFDTKLSAT |
| 361 | LYMVGFQIAS QVGQVIFSTP LGAFHDKMAQ ILPNNDMGSR |
| 401 | VTFWVISAIV LCALIYGFFV IKRDDQEVGG DPFYTDKQLR |
| 441 | QMEAAKA |

The sucrose transporter with SEQ ID NO:5 has only 39% sequence identity to SEQ ID NO:1 but is still a recognized sucrose transporter. Hence, the transporter employed can exhibit sequence variability compared to the sequences described herein.

For example, the sucrose transporter employed can have at least 60%, or at least 70%, or at least 80%, or at least 90%, or at least 95%, or at least 97%, or at least 98%, or at least 99% sequence identity to a sequence described herein.

In some cases, enzymes can have conservative changes such as one or more deletions, insertions, replacements, or substitutions that have no significant effect on the activities of the sucrose transporter.

The cyanobacteria are autotrophs that can thrive when cultured in a simple culture medium and exposed to light. Moreover, the cyanobacteria can produce organic nutrients (e.g., sugars) that can support the growth and functioning of organisms grown with the cyanobacteria. Organisms that are co-cultured with the cyanobacteria are referred to as heterotrophs because they cannot manufacture its own food and instead obtain nutrients and energy by taking in organic substances from the autotrophic cyanobacteria.

As illustrated herein, S. elongatus with a cscB⁺ transgene supports diverse heterotrophic microbes in co-culture, demonstrating that a flexible autotroph/heterotroph consortia platform can be employed for manufacture of useful products.

### Hydrogels

As described herein, the cyanobacteria can be encapsulated within a hydrogel matrix. There are benefits to embedding cyanobacteria within a matrix that includes the cyanobacteria and one or more types of hydrogels. As described herein, encapsulation of cyanobacteria greatly enhances stability, productivity, and modularity of co-cultures with heterotrophs, while also enabling selective recovery of heterotrophic biomass from the mixed cyanobacteria-heterotroph cultures. Encapsulation of cyanobacteria minimizes cell escape while still maintaining viability of trapped cells for more than five months. The hydrogel matrix did not degrade over more than five months of culture. Furthermore, encapsulation of cyanobacteria in a restrictive matrix can block predation by grazers, infection by cyanobacterial viruses, and slows division—greatly reducing the chance of loss of engineered pathways due to genetic mutation.

Examples of hydrogel materials that can be used include alginate, latex, silica, and combinations thereof. As illustrated herein, alginate forms an excellent encapsulation material that provides the benefits described in the foregoing paragraph. Latex is also a good encapsulation material. Latex is an easy-to-use, and inexpensive resin that has been used to embed bacteria. For example, one type of latex that can be used is Rhoplex SF012. Similarly, silica materials have superior properties for industrial use (e.g., thermostability). One example of a silica material that can be used is polyol silanes93.

The encapsulation procedure can include generation of beads or sheets of a diameter that allow diffusion of gases, nutrients, and other molecules to foster the stability and productivity of the cyanobacteria and the heterotroph cells. For example, the size and highly cross-linked network of encapsulation polymers may present a physical constraint on the diffusion of small molecules and can present a barrier to large molecules, such as proteins. The most salient molecules to consider are the permeability of CO₂ into the matrix, and escape of O₂ and sucrose. The cell density, percent alginate slurry, crosslinking efficiency, photosynthetic rates, and mixing rates are all variables that can influence the diffusion of substrates and products within the bead, so calculation of an optimal encapsulation diameter is non-trivial.

Optimal encapsulation diameters can vary and can include encapsulation diameters of about 0.05 mm to about 4 mm, or of about 0.075 mm to about 3.5 mm, or of about 0.1 mm to about 3 mm, or of about 0.15 mm to about 3 mm, or of about 0.15 mm to about 2.75 mm, or of about 0.25 mm to about 2.75 mm, or of about 0.2 mm to about 2 mm.

To evaluate or confirm optimal encapsulation diameters, titers of encapsulated cyanobacteria can be determined and compared to a control. The control can be non-encapsulated cyanobacteria or cyanobacteria encapsulated to a specified or desired diameter.

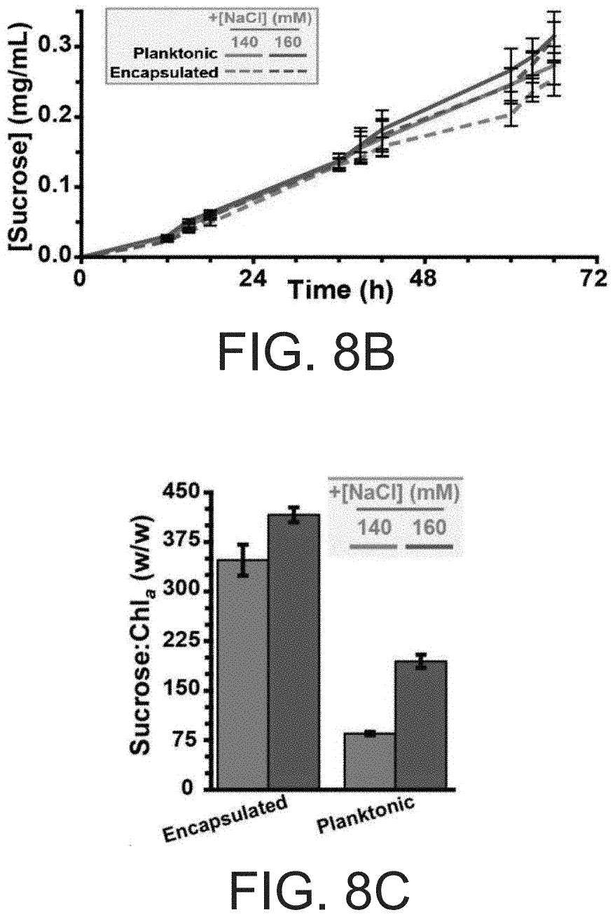

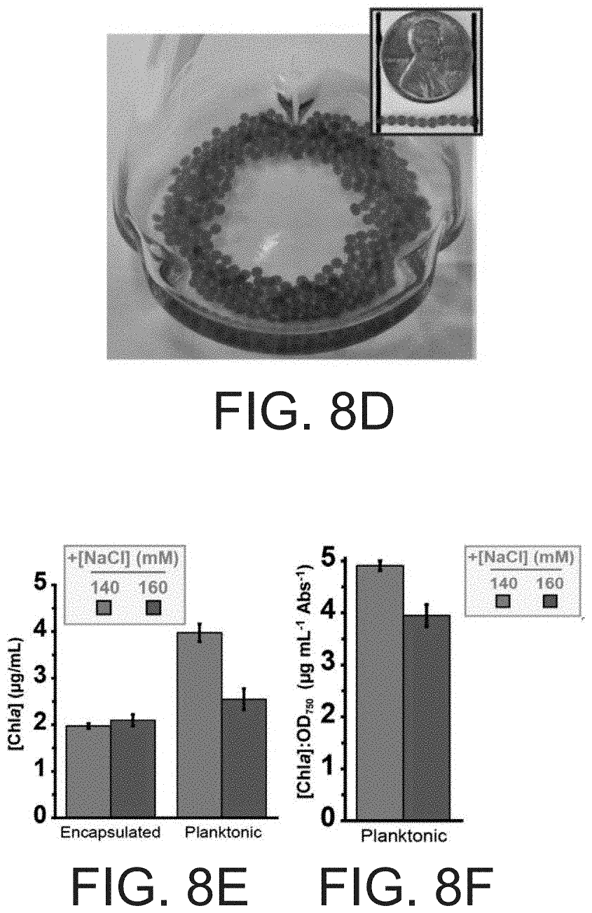

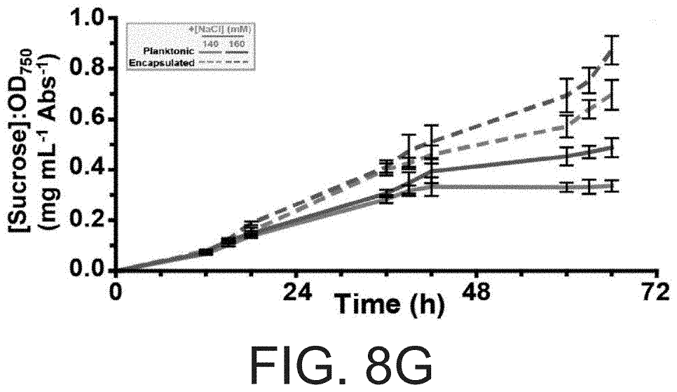

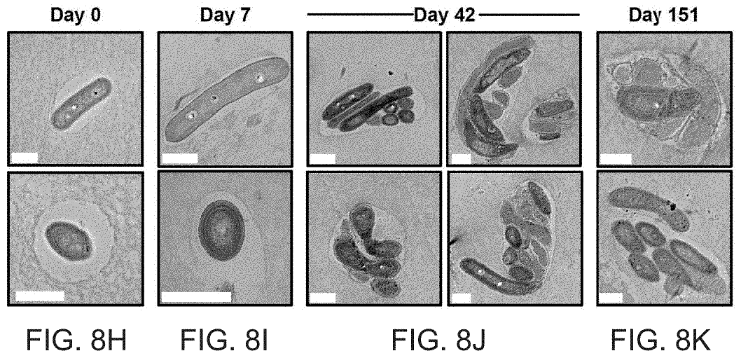

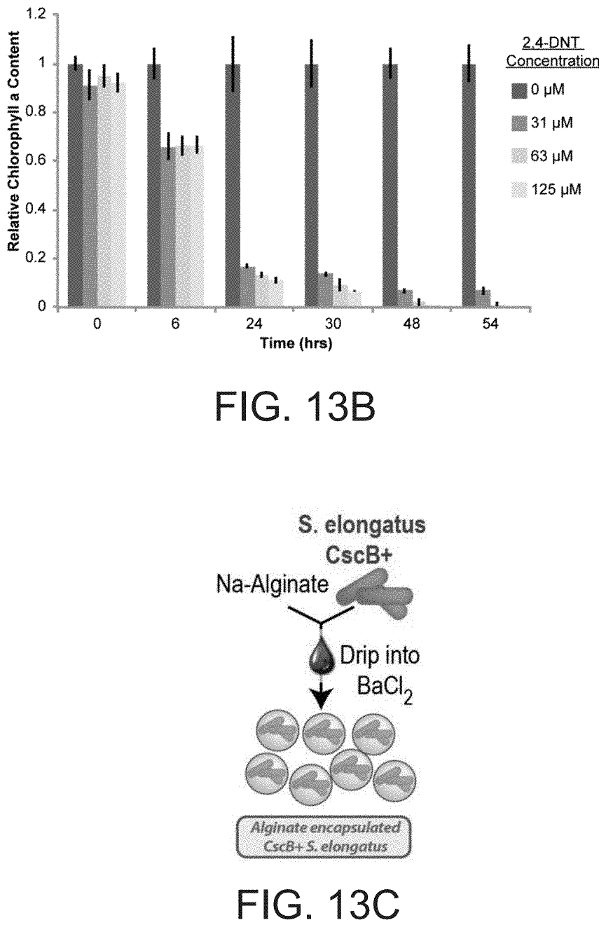

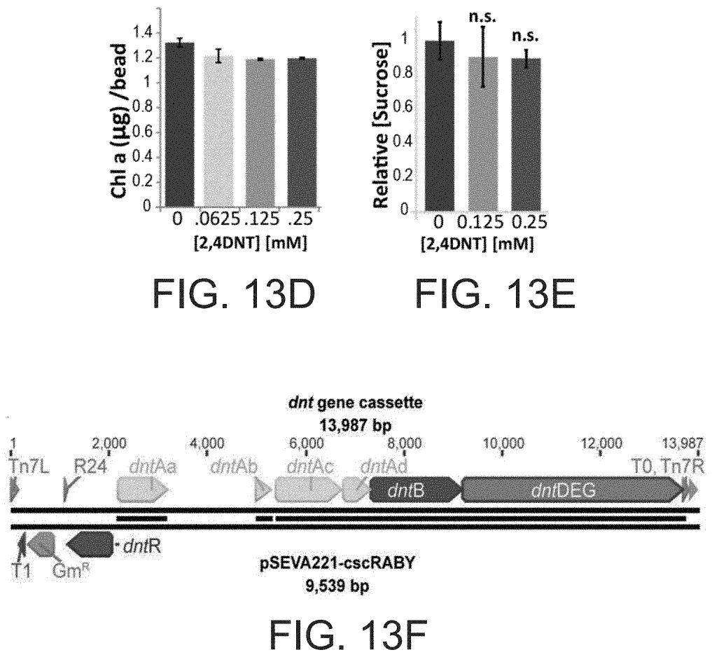

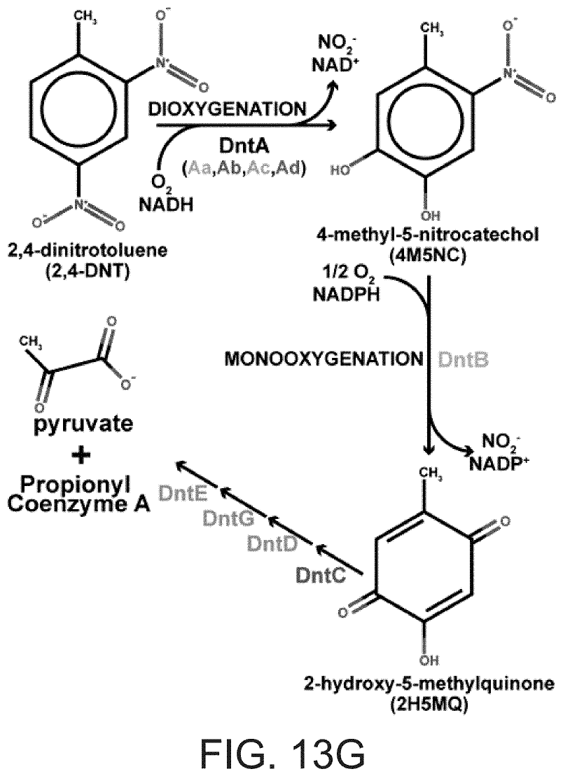

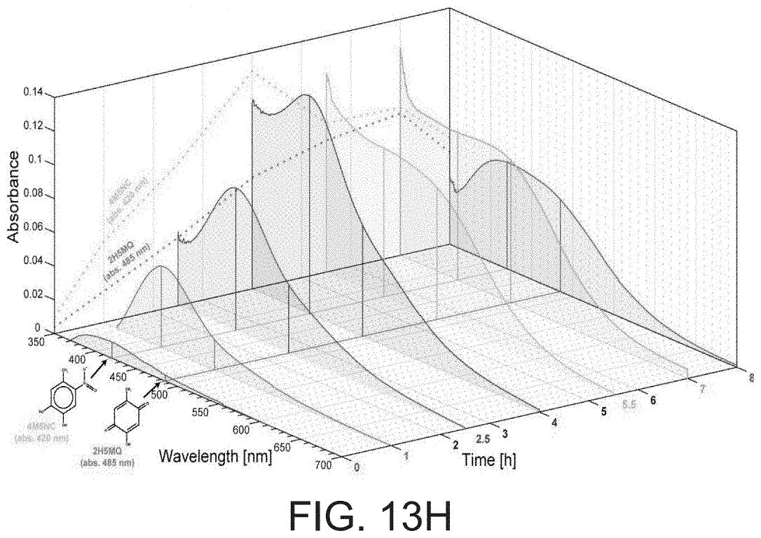

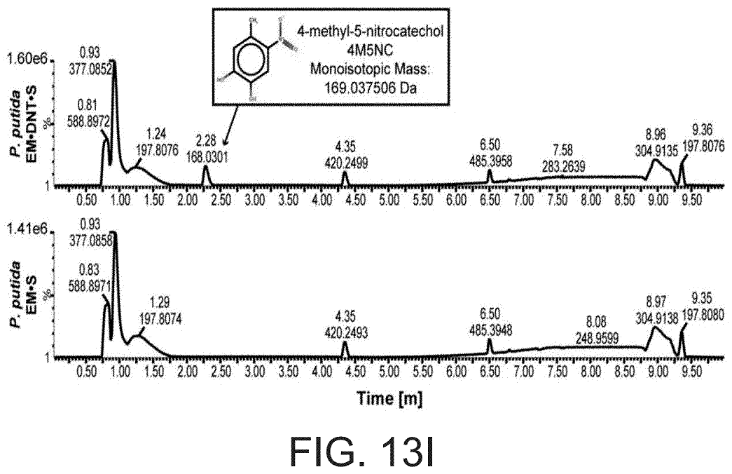

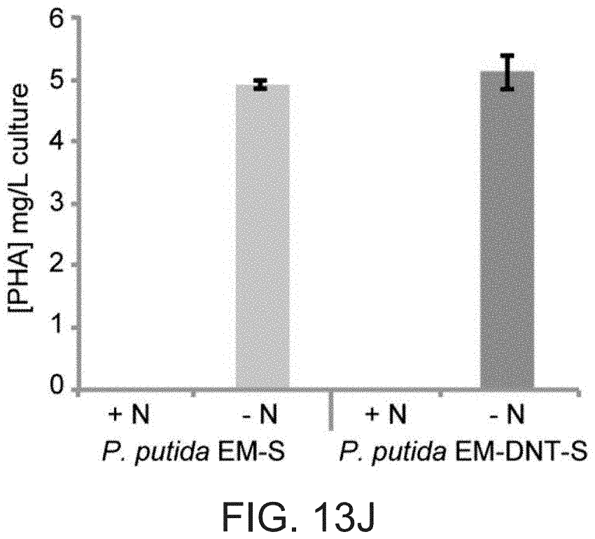

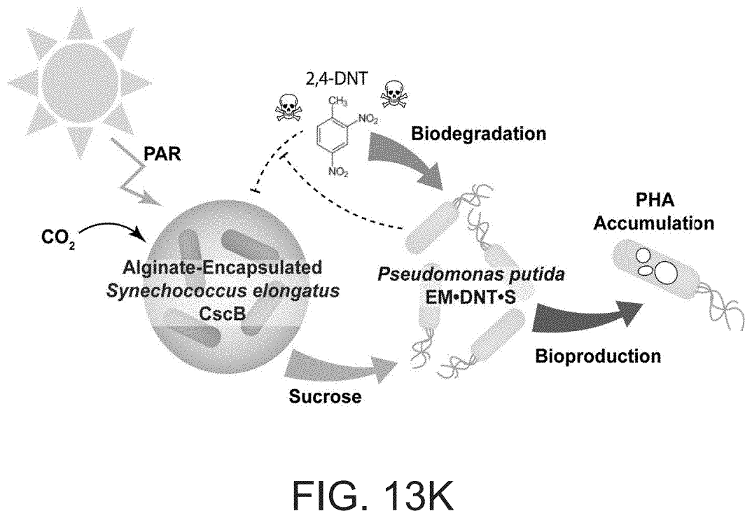

For example, the specific productivity of secreted sucrose and chlorophyll content of encapsulated cyanobacteria beads can be measured over time (see, e.g., FIG. 8C, 8G FIG. 13D, 13E) as a proxy for photosynthetic activity and cell viability. More precise measurements of photosynthetic parameters can be evaluated through monitoring chlorophyll a fluorescence dynamics embedded cells. These non-destructive methods can analyze the quantum efficiency of PSII, electron transfer rates, and PSI activity.

One method for encapsulation of cyanobacteria is to drip a mixture of at least one hydrogel with cyanobacteria cells into a curing solution. For example, a cyanobacteria cell/alginate suspension can be dripped into a barium chloride (BaCl) curing solution (see, e.g., FIG. 13C). This dripping method is relatively low-throughput and also does not allow for user control over the size or uniformity of the microdroplets that become encapsulated cyanobacteria.

Much higher volumes of encapsulated material can be generated with customizable bead sizes through the use of various commercially available equipment. For example, the encapsulation methods can include use of a Nisco Encapsulation Unit VAR V1, which uses electrostatically assisted spraying for rapid generation of large encapsulated bead volumes of user-specified sizes, for example, over the range of about 0.2 mm to 2 mm.

### Heterotrophs

A variety of heterotrophic microorganisms can be co-cultured with the cyanobacteria. Such heterotrophic microorganisms or heterotrophs can include bacteria, fungi, algae, and combinations thereof. For example, strains of Escherichia, Bacillus, Saccharormyces, Halomnonas, Pseudomnonas, or combinations are illustrated herein as examples of heterotrophs. Specific types of heterotrophs that can be employed include, for example, Escherichia coli, Bacillus subtilis, Saccharomyces cerevisiae, and combinations thereof.

Some synthetic communities have been constructed by cross-feeding of organisms [see, e.g., Song et al. Front Microbiol. 6:1298 (2015); Kim et al., Proc Natl Acad Sci. 105:18188-93 (2008); Wintermute & Silver, Genes Dev. 24:2603-14 (2010); Mee et al., Proc Natl Acad Sci USA 111:E2149-56 (2014); Song et al. Chem Soc Rev Chem Soc Rev. 6954:6954-81 (2014)]. However, sucrose is a metabolite that is naturally bioavailable to many microbes, and therefore the diversity of heterotrophic species with potential to be supported by cscB⁺ S. elongatus is broad.

For example, bacteria can be used as heterotrophic microorganisms that can be co-cultured with the cyanobacteria. Bacteria that naturally produce useful products, or bacteria that have been engineered to produce useful products are particularly useful as heterotrophs that can be co-cultured with the cyanobacteria.

In some cases, the heterotroph can be modified to reduce the function (activity) of a sucrose catabolism repressor cscR. As illustrated herein, E. coli with a deletion of the sucrose catabolism repressor cscR exhibit superior growth. One example of a CscR protein from the E. coli W strain has the following sequence (SEQ ID NO:6).

| | |
|---|---|
| 1 | LDLASDVCFC YSARFTANGL GMASLKDVAR LAGVSMMTVS |
| 41 | RVMHNAESVR PATRDRVLQA IQTLNYVPDL SARKMRAQGR |
| 81 | KPSTLAVLAQ DTATTPFSVD ILLAIEQTAS EFGWNSFLIN |
| 121 | IFSEDDAARA ARQLLAHRPD GIIYTTMGLR HITLPESLYG |
| 161 | ENIVLANCVA DDPALPSYIP DDYTAQYEST QHLLAAGYRQ |
| 201 | PLCFWLPESA LATGYRRQGF EQAWRDAGRD LAEVKQFHMA |
| 241 | TGDDHYTDLA SLLNAHFKPG KPDFDVLICG NDRAAFVAYQ |
| 281 | VLLAKGVRIP QDVAVMGFDN LVGVGHLFLP PLTTIQLPHD |
| 321 | IIGREAALHI IEGREGGRVT RIPCPLLIRC ST |

A nucleotide sequence encoding the CscR protein with SEQ ID NO:6 from the E. coli W strain, where the nucleotide sequence includes at least part of the natural promoter in the E. coli W strain is shown below at SEQ ID NO:7.

| | |
|---|---|
| 1 | TCAGGTGGAA CAACGGATCA ACAGCGGGCA AGGGATCCGC |
| 41 | GTCACTCTTC CCCCTTCACG ACCTTCAATA ATATGCAATG |
| 81 | CAGCTTCCCG CCCGATAATG TCATGTGGAA GCTGAATTGT |
| 121 | GGTCAGCGGC GGTAAAAACA GATGCCCGAC GCCAACCAGA |
| 161 | TTATCAAAGC CCATTACGGC GACATCCTGC GGGATTCGTA |
| 201 | CCCCCTTCGC CAGAAGAACC TGATAAGCCA CAAAGGCTGC |
| 241 | GCGATCGTTA CCACATATCA GAACATCAAA ATCTGGTTTG |
| 281 | CCCGGTTTGA AGTGGGCATT GAGTAAACTT GCGAGATCGG |
| 321 | TGTAGTGATC ATCACCTGTT GCCATGTGAA ATTGTTTCAC |
| 361 | CTCAGCCAGA TCTCGTCCAG CATCACGCCA GGCCTGCTCA |
| 401 | AATCCCTGCC GACGATACCC TGTTGCCAAC GCACTTTCCG |
| 441 | GTAGCCAGAA GCATAACGGT TGACGATAGC CCGCCGCGAG |
| 481 | CAAATGCTGT GTTGATTCAT ATTGTGCAGT GTAATCATCA |
| 521 | GGGATATAAC TGGGTAACGC TGGGTCATCC GCCACACAGT |
| 561 | TCGCCAATAC AATATTTTCA CCATACAGAG ACTCAGGCAG |
| 601 | CGTGATATGT CGCAGCCCCA TTGTAGTATA GATAATGCCA |
| 641 | TCCGGACGGT GGGCAAGCAG CTGACGTGCC GCGCGGGCAG |
| 681 | CGTCATCTTC AGAAAAAATA TTGATTAAAA AACTATTCCA |
| 721 | GCCGAACTCG CTGGCGGTTT GCTCAATGGC AAGCAGAATA |
| 761 | TCAACAGAGA AAGGAGTGGT AGCCGTGTCC TGCGCCAGCA |
| 801 | CGGCGAGAGT CGACGGCTTA CGTCCTTGAG CGCGCATCTT |
| 841 | ACGGGCGGAA AGATCAGGAA CATAATTCAG GGTCTGGATT |
| 861 | GCCTGCAATA CGCGGTCACG CGTTGCAGGA CGCACAGATT |
| 921 | CTGCATTATG CATCACCCGG GAGACTGTCA TCATCGACAC |
| 961 | TCCCGCCAGG CGTGCGACAT CCTTTAATGA AGCCATACCC |
| 1001 | AAGCCGTTTG CCGTAAAACG GGCACTGTAG CAGAAACAGA |
| 1041 | CGTCACTGGC GAGATCCAA |

Other types of bacteria, fungi, algae, and combinations thereof can also be heterotrophs that can be cultured with cyanobacteria.

The heterotrophs can express a variety of useful products. Examples include drugs, enzymes, nutrients, proteins, oils, carbohydrates, alcohols, fatty acids, vitamins, pigments, pharmaceutical enzymes, biotechnological enzymes, hydrogen gas, polymer substrates/monomers, polymers, biofuels, metabolites, and combinations thereof. In some cases, the heterotrophs can perform useful functions such as metabolizing undesirable compounds or materials (e.g., pollutants), sequestering useful materials or compounds, and the like.

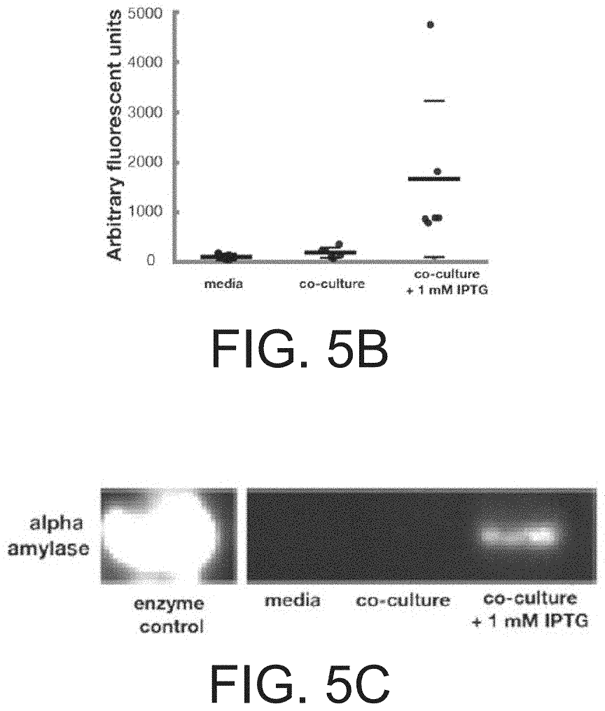

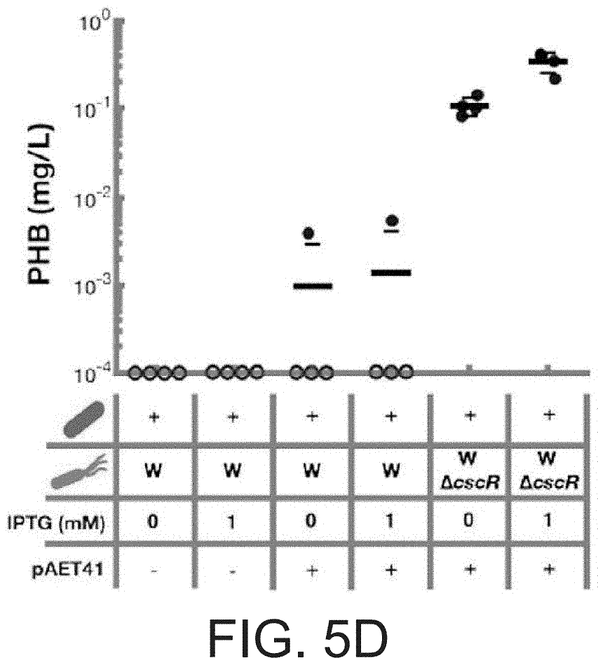

For example, as illustrated herein B. subtilis strain 168 naturally produces and secretes alpha-amylase (FIG. 5B-5C). Also as illustrated herein, transgenic E. coli is capable of making polyhydroxybutyrate (PHB) when carrying the pAET41 plasmid and co-cultured with cscB+ S. elongatus (see. e.g., FIG. 5D).

The enzymes involved in synthesis of polyhydroxybutyrate (P1B) include those encoded by phbA, phbB, and phbC genes (see, e.g., Peoples et al., J. Biol. Chem. 264 (26): 15293-15297 (1989)). These genes can be present in a variety of bacterial species such as Alcaligenes eutrolropus, Burkholderia pseudoinallei, Cupravidus necator, Ralstonia eutropha, or Zoogloea ramigera.

The phbA gene encodes a beta-thiolase that can, for example, have the following sequence (SEQ ID NO:8 from Zoogloea ranigera).

| | |
|---|---|
| 1 | MSTPSIVIAS ARTAVGSFNG AFANTPAHEL GATVISAVLE |
| 41 | RAGVAAGEVN EVILGQVLPA GEGQNPARQA AMKAGVPQEA |
| 81 | TAWGMNQLCG SGLRAVALGM QQIATGDASI IVAGGMESMS |
| 121 | MAPHCAHLAG GVKMGDFKMI DTMIKDGLTD AFYGYHMGTT |
| 161 | AENVAKQWQL SRDEQDAFAV ASQNKAEAAQ KDGRFKDEIV |
| 201 | PFIVKGRKGD ITVDADEYIR HGATLDSMAK LRPAFDKEGT |
| 241 | VTAGNASGLN DGAAAALLMS EAEASRRGIQ PLGRIVSWAT |
| 281 | VGVDPKVMGT GPIPASRKAL ERAGWKIGDL DLVEANEAFA |
| 321 | AQACAVNKDL GWDPSIVNVN GGAIAIGHPI GASGARILNT |
| 361 | LLFEMKRRGA RKGLATLCIG GGMGVAMCIE SL |

The phbB gene encodes a NADP-specific acetoacetyl-CoA reductase that can, for example, have the following sequence (SEQ ID NO:9 from Ralstonia eutropha).

| | |
|---|---|
| 1 | MTQRIAYVTG GMGGIGTAIC QRLAKDGFRV VAGCGPNSPR |
| 41 | REKWLEQQKA LGFDFIASEG NVADWDSTKT AFDKVKSEVG |
| 81 | EVDVLINNAG ITRDVVFRKM TRADWDAVID TNLTSLFNVT |
| 121 | KQVIDGMADR GWGRIVNISS VNGQKGQFGQ TNYSTAKAGL |
| 161 | HGFTMALAQE VATKGVTVNT VSPGYIATDM VKAIRQDVLD |
| 201 | KIVATIPVKR LGLPEEIASI CAWLSSEESG FSTGADFSLN |
| 241 | GGLHMG |

The phbC gene encodes a PHB polymerase that can, for example, have the following sequence (SEQ ID NO:10 from Cupriavidus necator).

| | |
|---|---|
| 1 | MATGKGAAAS TQEGKSQPFK VTPGPFDPAT WLEWSRQWQG |
| 41 | TEGNGHAAAS GIPGLDALAG VKIAPAQLGD IQQRYMKDFS |
| 81 | ALWQAMAEGK AEATGPLHDR RFAGDAWRTN LPYRFAAAFY |
| 121 | LLNARALTEL ADAVEADAKT RQRIRFAISQ WVDAMSPANF |
| 161 | LATNPEAQRL LIESGGESLR AGVRNMMEDL TRGKISQTDE |
| 201 | SAFEVGRNVA VTEGAVVFEN EYFQLLQYKP LTDKVHARPL |
| 241 | LMVPPCINKY YILDLQPESS LVRHVVEQGH TVFLVSWRNP |
| 281 | DASMAGSTWD DYIEHAAIRA IEVARDISGQ DKINVLGFCV |
| 321 | GGTIVSTALA VLAARGEHPA ASVTLLTTLL DFADTGILDV |
| 361 | FVDEGHVQLR EATLGGGAGA PCALLRGLEL ANTFSFLRPN |
| 401 | DLVWNYVVDN YLKGNTPVPF DLLFWNGDAT NLPGPWYCWY |
| 441 | LRHTYLQNEL KVPGKLTVCG VPVDLASIDV PTYIYGSRED |
| 481 | HIVPWTAAYA STALLANKLR FVLGASGHIA GVINPPAKNK |
| 521 | RSHWTNDALP ESPQQWLAGA IEHHGSWWPD WTAWLAGQAG |
| 561 | AKRAAPANYG NARYRAIEPA PGRYVKAKA |

In another example, an industrial pollutant that is of increasing concern to the U.S. Environmental Protection Agency (EPA) is the compound 2,4-dinitrotolulene (2,4-DNT; EPA. Emerging Contaminants—Dinitrotoluene (DNT)—EPA.; 2014. https://www.epa.gov/sites/production/files/2014-03/documents/ffrrofactsheet-contaminant-dnt_january2014_final.pdf). A byproduct of manufacturing of polyurethane polymers, explosives, and plasticizing agents, 2,4-DNT is classified as toxic to most organisms and is increasingly detected in ground water and soils at levels above those regarded as safe. Exposure to 2,4-DNT causes damage to the central nervous system, the circulatory system, and is associated with liver damage and oncogenes (Tchounwou et al. Rev Env Heal. 18(3):203-229 (2003)). Therefore, the EPA classifies 2,4-DNT as a priority pollutant.

One effective method for the remediation of environmental 2,4-DNT is through the use of microbial strains capable of degrading 2,4-DNT and other nitrotoluene derivatives (Han et al., Chemosphere. 85(5):848-853 (2011); Wang et al., J Appl Microbiol. 110(6):1476-1484 (2011); Aburto-Medina et al., Appraising the Role of Microorganisms. In: Enhancing Cleanup of Environmental Pollutants. Springer International Publishing; 2017:5-20 (2017)).

One such strain that has natural capacities to metabolize diverse aromatic compounds is Pseudomonas putida. While P. putida naturally exhibits some capacity to degrade recalcitrant aromatic compounds, it does not degrade 2,4-DNT. However, as described herein Pseudomonas putida can be engineered to express enhanced 2,4-DNT degradation capabilities such as the enzymes DntA, DntB, DntC, DntD, DntG, DntE, or combinations thereof. Such DntA, DntB, DntC, DntD, DntG, and DntE enzymes are expressed, for example, by Burkholderia (Pdrez-Pantoja et al., PLoS Genet. 9(8) (2013)). For example, 2,4-DNT can be degraded into non-toxic products (e.g., pyruvate and propionyl-CoA) by such enzymes as illustrated by the metabolic pathway shown below.

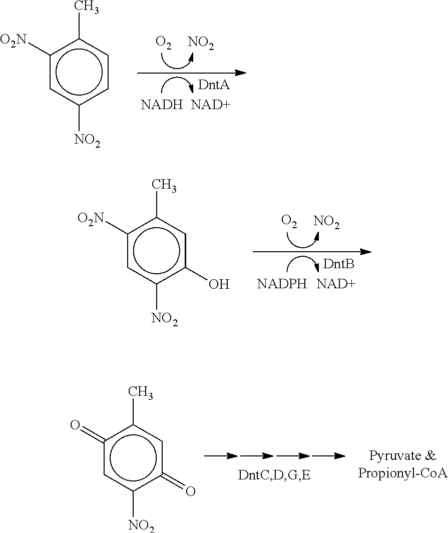

The DntA enzyme is a DNT dioxygenase that hydroxylates the aromatic ring in positions 4 and 5 to yield 4-methyl-5-nitrocatechol, releasing, at the same time, the first nitro substituent. The substituted catechol is subsequently mono-oxygenated by the DntB enzyme, which is a hydroxylase; this step eliminates the remaining nitro group in the structure, thereby producing 2-hydroxy-5-methylquinone. The rest of the pathway (executed by DntCDGE) includes a ring cleavage reaction and channeling of the products towards the central metabolism, in which they are finally metabolized.

The DntA gene encodes a 2,4-DNT dioxygenase that can, for example, have the following sequence (SEQ ID NO:11 from Burkholderia sp. DNT).

| | |
|---|---|
| 1 | MSENWIDAAA RDEVPEGDVI GINIVGKEIA LYEVAGEIYA |
| 41 | TDNTCTHGAA RMSDGFLEGR EIECPLHQGR FDVCTGKALC |
| 81 | TPLTQDIKTY PVKIENMRVM LKLD |

The DntB gene encodes a hydroxylase that can, for example, have the following sequence (SEQ ID NO:12 from Burkholderia).

| | |
|---|---|
| 1 | MVDFKTYFEL LNLYSDYAMV CDSANWEKWP DFFIETGTYR |
| 41 | LQPRENFEQD LPLCLLALES KAMIRDRVYG VKETMYHDPY |
| 81 | YQRHIVGTPR VLSVERDADG ERITAEASYA VIRTKYDGDS |
| 121 | TIFNAGYYRD VIVRTPEGLK LKSRLCVYDS EMIPNSIIYP |
| | I |

DntCDGE provides ring cleavage and other functions and can, for example, have the following sequence (SEQ ID NO:13 from Burkholderia sp. BC1).

| | |
|---|---|
| 1 | MAEIVAGFML PHDPLIASIP DAPPLQKRET CMAAYAAIVE |
| 41 | RIKDLKVDTV IVIGDDHYTM HSPACIPRCL IGIGDVEGPR |
| 81 | EEWLGIPRAK IENNEALAHH IMQTGFDVGV DWAVAKTLVI |
| 121 | DHSTTIPIHY AVRPAAGVRA IPVYLNTGFE PLISSRRAYQ |
| 161 | IGKVIGEAVA SWSGTERVAI YGTGGLSHWP GMAQMGKVNA |
| 201 | EWDRKILSHV EEGNVEALIA LTDEEILRDG GNGGLEIKNW |
| 241 | ICAMGALGTV RGELIAYESV PEWVCGCGYL EMKRAA |

Species of bacteria such as Pseudomonas putida can be used as a heterotrophic organism, particularly after modification to include expression cassettes encoding enzymes such as the DntA, DntB, DntC, DntD, DntG, and/or DntE enzymes using recombinant methods.

In addition, Pseudomonas putida can be successfully co-cultivated with S. elongatus CscB+(Lowe et al. Biotechnol Biofuels. 10(1):190 (2017)). Hence, the co-culture mixtures and methods described herein can be used for light-driven bioproduction, and also for light-catalyzed bioremediation of toxic aromatics.

Therefore, encapsulated cyanobacterial cells can secrete sucrose, while allowing the P. putida strains to grow on the secreted sucrose. P. putida can grow in co-culture with S. elongatus CscB+. Optimized media are described herein that support growth of many heterotrophic species in co-culture with S. elongatus CscB+. Engineered strains of P. putida (e.g., derivatives of strain KT2440) having, for example, genomically integrated dntABCDEG genes from Burkholderia can breakdown 2,4-DNT while providing core carbon intermediates that can be utilized for cell growth. Furthermore, these strains also express cscAB genes that improve the utilization of sucrose.

In another example, the heterotroph can express enzymes that can facilitate manufacture of indole. Indole is the parent compound for the synthesis of a number of natural products and pharmaceuticals. Natural occurring indoles with physiological roles in humans include, for example, the neurotransmitter serotonin and the hormone melatonin. Some indole-derived compounds with pharmacological significance include sumatriptan, ondnsetron, alosetron, 2-aroylindole, 2-aryl-3arylcorbonylindole, indolyl-3-glycoxamide, ellipticine, mycotoxin gliotoxin. A number of indole-containing compounds have been utilized in drug therapies for humans including Vincristine, Roxindole, Atevirdine, Proamanullin, Reserpine, Pindolol, Oglufanide. Hence, by employing the co-culture and/or the encapsulation methods described herein, products such as indole can be made more efficiently and at lower cost.

For example, as illustrated herein, E. coli that can transgenically express tryptophanase and that are co-cultured with cyanobacteria can produce indole. One example of a sequence for a tryptophanase enzyme is shown below as SEQ ID NO:14.

| | |
|---|---|
| 1 | MENFKHLPEP FRIRVIEPVK RTTRAYREEA IIKSGMNPFL |
| 41 | LDSEDVFIDL LTDSGTGAVT QSMQAAMMRG DEAYSGSRSY |
| 81 | YALAESVKNI FGYQYTIPTH QGRGAEQIYI PVLIKKREQE |
| 121 | KGLDRSKMVA FSNYFFDTTQ GHSQINGCTV RNVYIKEAFD |
| 161 | TGVRYDFKGN FDLEGLERGI EEVGPNNVPY IVATITSNSA |
| 201 | GGQPVSLANL KAMYSIAKKY DIPVVMDSAR FAENAYFIKQ |
| 241 | REAEYKDWTI EQITRETYKY ADMLAMSAKK DAMVPMGGLL |
| 281 | CMKDDSFFDV YTECRTLCVV QEGFPTYGGL EGGAMERLAV |
| 321 | GLYDGMNLDW LAY |

The tryptophanase need not have the foregoing sequence (SEQ ID NO:14). Tryptophanases with a variety of different sequences can be employed. For example, the tryptophanase can have a sequence such as SEQ ID NO:15 from Sediminispirochaeta smaragdinae.

| | |
|---|---|
| 1 | MAITYLPEPF RIKMVETIKM LTPEEREQRI AEAKYNLFNL |
| 41 | KGKDVYIDLL TDSGTNAMSQ EQWAGVMKGD EAYAGGASYY |
| 81 | KLLEAAQDIT GYAYIQPVHQ GRAAEKVLFG ILMGKGQFSI |
| 121 | SNMHFDTTRA HVELTGARAI DCVVSDAMDP AKRAPFKGNM |
| 161 | DVDKLESLIK KYGAEKVALV VMTVTNNSAG GQPVSMQNIR |
| 201 | ETASVCKKYN IKFCIDAARF AENAFFIKKR EDGYRDASIK |
| 241 | DITNEMFSYA DMFTMSAKKD AIVNMGGLIG IKDDAELFSA |
| 281 | CKGRTISFEG FITYGGLSGR DLESLAIGLY EGIDENQLRY |
| 321 | RIGQIEYLAS RLDDAGITYQ APAGGHGIFV DAKALLPHIP |
| 361 | YYEFPGQALA VELYKEAGIR TCDIGSYMLG NDPDTGKQLH |
| 401 | AEFEFTRLAI PRRVYTQAHI DVMAEALIEI KKRANRVKGY |
| 441 | KITWEPPVLR HFQASLKPLT |

As shown below, the tryptophanases with SEQ ID NOs: 14 and 15 share about 46% sequence identity.

| | | |
|---|---|---|
| Seq14 | 6 | HLPEPFRIRVIEPVKRTTRAYREEAIIKSGMNPFLLDSEDVFIDLLTDSGTGAVTQSMQA |
| Seq15 | 5 | YLPEPFRIKMVETIKMLTPEEREQRIAEAKYNLFNLKGKDVYIDLLTDSGTNAMSQEQWA |
| | | ******* * * * * ** * * * * * * ********* * * * |
| Seq14 | 66 | AMMRGDEAYSGSRSYYALAESVKNIFGYQYTIPTHQGRGAEQIYIPVLIKKREQEKGLD |
| Seq15 | 65 | GVMKGDEAYAGGASYYKLLEAAQDITGYAYIQPVHQGRAAEKVLFGILMGKGQ------- |
| | | * ***** * *** * * * ** * * **** ** * * |
| Seq14 | 126 | SKMVAFSNYFFDTTQGHSQINGCTVRNVYIKEAFDTGVRYDFKGNFDLEGLERGIEEVGP |
| Seq15 | 118 | ---FSISNMHFDTTRAHVELTGARAIDCVVSDAMDPAKRAPFKGNMDVDKLESLIKKYGA |
| | | ** **** * * ** * * * **** * ** * * |
| Seq14 | 186 | NNVPYIVATITSNSAGGQPVSLANLKAMYSIAKKYDIPVVMDSARFAENAYFIKQREAEY |
| Seq15 | 175 | EKVALVVMTVTNNSAGGQPVSMQNIRETASVCKKYNIKFCIDAARFAENAFFIKKREDGY |
| | | * * * * ********* * * *** * * * ****** *** ** * |
| Seq14 | 246 | KDWTIEQITRETYKYADMLAMSAKKDAMVPMGGLLCMKDDSFFDVYTECRTLCVVQEGFP |
| Seq15 | 235 | RDASIKDITNEMFSYADMFTMSAKKDAIVNMGGLIGIKDDA--ELFSACKGRTISFEGFI |
| | | * * ** * **** ******* * **** *** * *** |
| Seq14 | 306 | TYGGLEGGAMERLAVGLYDGMNLDWLAY |
| Seq15 | 293 | TYGGLSGRDLESLAIGLYEGIDENQLRY |
| | | ***** * * ** **** * * |

The heterologous functions employed herein by the cyanobacteria and the heterotrophic organism (e.g., any of the enzymes transgenes and/or expression cassettes) can exhibit sequence variability compared to the sequences described herein. For example, any of the enzymes, transgenes and/or expression cassettes employed can have at least 60%, or at least 70%, or at least 80%, or at least 90%, or at least 95%, or at least 97%, or at least 98%, or at least 99% sequence identity to a sequence described herein.

In some cases, enzymes can have conservative changes such as one or more deletions, insertions, replacements, or substitutions that have no significant effect on the activities of the enzymes. Examples of conservative substitutions are provided below.

| Type of Amino Acid | Substitutable Amino Acids |
|---|---|
| Hydrophilic | Ala, Pro, Gly, Glu, Asp, Gln, Asn, Ser, Thr |
| Sulfhydryl | Cys |
| Aliphatic | Val, Ile, Leu, Met |
| Basic | Lys, Arg, His |
| Aromatic | Phe, Tyr, Trp |

In some cases, the heterotroph can be a fungal or yeast strain such as Saccharomyces cerevisiae, Saccharomyces uvarum (also known as Saccharomyces carlsbergensis), Saccharomyces pastorianus, or other strains. Such fungal or yeast strains can be modified to take up sugars and/or carbohydrates more efficiently. For example, S. cerevisiae W303^{Clump} (originally called Recreated02 in Koschwanez et al. 2013) which contain mutations in genes CSE2, IRA1, MTH1, and UBR1 that enhance fitness in dilute sucrose.

In constructing these consortia, unforeseen interactions were observed that were shared across different heterotrophic species. For example, light-driven processes of cyanobacteria have negative impacts on all tested heterotrophic species while, conversely, growth of all heterotrophic species simulates cyanobacterial growth. However, as illustrated herein, the negative impact was photosynthetically produced oxygen by cyanobacteria when exposed to light. Such negative impacts on heterotroph growth are controlled by a variety of processes (e.g., sparging with low-oxygen containing gases or adding anti-oxidants). By taking measures to mitigate deleterious interactions, the inventors could stabilize consortia over time. Experiments described herein demonstrate that consortia persist in the face of fluctuations in light availability, population density, and composition. These consortia can be functionalized to produce target compounds, where the end product is dictated by the heterotrophic partner.

### Expression of Enzymes

Also described herein are expression systems that include at least one expression cassette (e.g., expression vectors or transgenes) that encode one or more of the enzyme(s) described herein. These expression systems can be present in the cyanobacteria or in the heterotroph. The expression systems can also include one or more expression cassettes encoding an enzyme that can synthesize or degrade a product.

Cells containing such expression systems are further described herein. The cells containing such expression systems can be used to manufacture the enzymes (e.g., for in vitro use) or products produced by the enzymes. Methods of using the enzymes or cells containing expression cassettes encoding such enzymes to make products, degrade products, and combinations thereof are also described herein.

Nucleic acids encoding the enzymes can have sequence modifications. For example, nucleic acid sequences described herein can be modified to express enzymes that have modifications. Most amino acids can be encoded by more than one codon. When an amino acid is encoded by more than one codon, the codons are referred to as degenerate codons. A listing of degenerate codons is provided in the table below.

Degenerate Amino Acid Codons

| Amino Acid | Three Nucleotide Codon |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

Different organisms may translate different codons more or less efficiently (e.g., because they have different ratios of tRNAs) than other organisms. Hence, when some amino acids can be encoded by several codons, a nucleic acid segment can be designed to optimize the efficiency of expression of an enzyme by using codons that are preferred by an organism of interest. For example, the nucleotide coding regions of the enzymes described herein can be codon optimized for expression in various cyanobacterial or heterotroph species.

An optimized nucleic acid can have less than 98%, less than 97%, less than 95%, or less than 94%, or less than 93%, or less than 92%, or less than 91%, or less than 90%, or less than 89%, or less than 88%, or less than 85%, or less than 83%, or less than 80%, or less than 75% nucleic acid sequence identity to a corresponding non-optimized (e.g., a non-optimized parental or wild type enzyme nucleic acid) sequence.

The enzymes described herein can be expressed from an expression cassette and/or an expression vector. Such an expression cassette can include a nucleic acid segment that encodes an enzyme operably linked to a promoter to drive expression of the enzyme. Convenient vectors, or expression systems can be used to express such enzymes. In some instances, the nucleic acid segment encoding an enzyme is operably linked to a promoter and/or a transcription termination sequence. The promoter and/or the termination sequence can be heterologous to the nucleic acid segment that encodes an enzyme. Expression cassettes can have a promoter operably linked to a heterologous open reading frame encoding an enzyme. The invention therefore provides expression cassettes or vectors useful for expressing one or more enzyme(s).

Constructs (e.g., expression cassettes) and vectors comprising the isolated nucleic acid molecule, e.g., with optimized nucleic acid sequence, as well as kits comprising the isolated nucleic acid molecule, construct or vector are also provided.

Nucleic acids encoding one or more enzyme(s) can have one or more nucleotide deletions, insertions, replacements, or substitutions. For example, the nucleic acids encoding one or more enzyme(s) can, for example, have less than 95%, or less than 94.8%, or less than 94.5%, or less than 94%, or less than 93.8%, or less than 94.50% nucleic acid sequence identity to a corresponding parental or wild-type sequence. In some cases, the nucleic acids encoding one or more enzyme(s) can have, for example, at least 50%, or at least 55%, or at least 60%, or at least 65%, or at least 70%, or at least 75%, or at least 80%, or at least 85%, or at 90% sequence identity to a corresponding parental or wild-type sequence. Examples of parental or wild type nucleic acid sequences for unmodified enzyme(s) with amino acid sequences SEQ ID NOs:1, 3, 5, 6, 8, 9, 10, 11, 12, or 13, and nucleic acid sequences SEQ ID NOs:2, 4, 7, 9, 11, 14, 16-24, or 25. Any of these nuclei acid or amino acid sequences can, for example, encode or have sequences with less than 99.5%, less than 99%, less than 98%, less than 97%, less than 96%, less than 95%, less than 94.8%, less than 94.5%, less than 94%, less than 93.8%, less than 93.5%, less than 93%, less than 92%, less than 91%, or less than 90% sequence identity to a corresponding parental or wild-type sequence.

A variety of promoters can be included in the transgenes, expression cassettes and/or expression vectors. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences can also contain regulatory sequences such as enhancer sequences that can influence the level of gene expression. Some isolated promoter sequences can provide for gene expression of heterologous DNAs, that is a DNA different from the native or homologous DNA.

Promoters can be strong or weak, or inducible. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. A strong promoter for heterologous DNAs can be advantageous because it provides for a sufficient level of gene expression for easy detection and selection of transformed cells and provides for a high level of gene expression when desired. In some cases, the promoter within such expression cassettes/vectors can be functional during plant development or growth.

The promoter can be a promoter functional in a heterotrophic organism or a cyanobacteria such as a bacterial promoter, yeast promoter, viral promoter, or a mammalian promoter. The promoter can be a heterologous promoter.

As used herein, “heterologous” when used in reference to a gene or nucleic acid refers to a gene, nucleic acid, or enzyme that has been manipulated in some way. For example, a heterologous promoter is a promoter that contains sequences that are not naturally linked to an associated coding region. Thus, a heterologous promoter is not the same one as the natural promoter that drives expression of an operably linked coding region.

Examples of promoters that can be used include, but are not limited to, the T7 promoter (e.g., optionally with the lac operator), the CaMV 35S promoter (Odell et al., Nature. 313:810-812 (1985)), the CaMV 19S promoter (Lawton et al., Plant Molecular Biology. 9:315-324 (1987)), nos promoter (Ebert et al., Proc. Natl. Acad. Sci. USA. 84:5745-5749 (1987)), Adh1 promoter (Walker et al., Proc. Natl. Acad. Sci. USA. 84:6624-6628 (1987)), sucrose synthase promoter (Yang et al., Proc. Natl. Acad. Sci. USA. 87:4144-4148 (1990)), α-tubulin promoter, ubiquitin promoter, actin promoter (Wang et al., Mol. Cell. Biol. 12:3399 (1992)), cab (Sullivan et al., Mol. Gen. Genet. 215:431 (1989)), PEPCase promoter (Hudspeth et al., Plant Molecular Biology. 12:579-589 (1989)), the CCR promoter (cinnamoyl CoA:NADP oxidoreductase, EC 1.2.1.44) isolated from Lollium perenne, (or a perennial ryegrass) and/or those associated with the R gene complex (Chandler et al., The Plant Cell. 1:1175-1183 (1989)).

Other constitutive or inducible promoters can be used with or without associated enhancer elements. Examples include a baculovirus derived promoter, the p10 promoter. Although some heterotrophs are bacteria or yeast, cyanobacteria and heterotrophs may also employ plant promoters.

Expression cassettes that include a promoter operably linked to a nucleic acid segment encoding a polypeptide or peptide can include other elements such as a segment encoding 3′ nontranslated regulatory sequences, and restriction sites for insertion, removal and manipulation of segments of the expression cassettes. The 3′ nontranslated regulatory DNA sequences can act as a signal to terminate transcription and in some cases can allow for the polyadenylation of the resultant mRNA. The 3′ nontranslated regulatory DNA sequence preferably includes from about 300 to 1,000 nucleotide base pairs and contains prokaryotic or eukaryotic transcriptional and translational termination sequences. Various 3′ can be employed. For example, such 3′ nontranslated regulatory sequences can be obtained as described in An (Methods in Enzymology. 153:292 (1987)). Many such 3′ nontranslated regulatory sequences are also present in plasmids available from commercial sources such as Clontech, Palo Alto, Calif.

### Culture Methods

Cyanobacteria can be cultured in a variety of simple media in the presence of light. For example, cyanobacteria can be grown in sea water. In some cases, cyanobacteria can be cultured in a medium that contains a nitrate salt, a phosphate salt, a magnesium salt, a calcium salt, a carbonate salt, a chelator, citric acid, ferric ammonium, and combinations thereof. Cyanobacteria are often cultured at about neutral pH or in slightly alkaline culture media. One example of a cyanobacteria medium can contain the following components.
Sodium nitrate 1.500 g/liter
Dipotassium hydrogen phosphate 0.0314 g/liter
Magnesium sulphate 0.036 g/liter
Calcium chloride dihydrate 0.0367 g/liter
Sodium carbonate 0.020 g/liter
Disodium magnesium EDTA 0.001 g/liter
Citric acid 0.0056 g/liter
Ferric ammonium citrate 0.006 g/liter
Final pH after sterilization (at 25° C.) 7.1
   Some cyanobacteria culture media are available commercially. One example, is the BG-11 media (Sigma-Aldrich). Such a BG-11 media can be supplemented with a buffer, such as 1 g/L HEPES. For example, S. elongatus can readily be grown and maintained in BG-11 media supplemented with 1 g/L HEPES, at pH 8, in constant light at 35° C.

When cyanobacteria are co-cultured with heterotrophs, the cyanobacteria culture media can be supplemented with a source of nitrogen, a buffer, electrolytes, an alkali, an acid, or a combination thereof.

As described herein, co-culture media have been optimized for growth and maintenance of either prokaryotes (referred to as ^{CoB}BG-11 medium) or fungi such as yeast (referred to as ^{CoY}BG-11 medium). ^{CoB}BG-11 consists of BG-11 medium supplemented with 106 mM NaCl, 4 mM NH₄Cl and 25 mM HEPPSO, pH 8.3-KOH. Indole (100 μM) can be added to the ^{CoB}BG-11 medium when co-culturing some types of heterotrophs such as B. subtilis 168.

The ^{CoY}BG-11 medium consists of BG-11 medium supplemented with 0.36 g/L Yeast Nitrogen Base without amino acids (Sigma Aldrich), 106 mM NaCl, 25 mM HEPPSO, pH 8.3-KOH and 1 mM KPO3.

Bacteria can be co-cultured on solid co-culture plates that can include ^{CoB}BG-11 media with 1% autoclaved agar.

As illustrated herein, the atmosphere or the culture media in which a consortium of cyanobacteria and heterotrophic cells is cultured can be modulated to reduce the inhibitory effects of such oxygen production. For example, the consortium can be sparged with low-oxygen containing gases or anti-oxidants can be added to the culture medium.

In some cases, the cultures can be sparged or incubated with a gas having less than 20% oxygen, or less than 17% oxygen, or less than 15% oxygen, or less than 12% oxygen, or less than 10% oxygen, or less than 7% oxygen, or less than 5% oxygen, or less than 3% oxygen, or less than 1% oxygen. In some cases, the cultures can be sparged with a gas that is devoid of oxygen (e.g., 12:10:82 H₂:CO₂:N₂).

In some cases, antioxidants such as DCMU (3-(3,4-dichlorophenyl)-1,1-dimethylurea), thiosulfate, or a combination thereof can be used in the culture medium. Concentrations of such antioxidants can vary from about 1 μM to 1000 mM, or about 10 μM to 200 mM.

The following Examples describe some experimental work performed during development of the invention.

### Example 1: Materials and Methods

This Example describes some of the materials and methods used in the development of aspects of the invention.

### Strains, Media, and Axenic Characterization

S. elongatus PCC 7942 (obtained from ATCC #33912) was engineered to secrete sucrose through the expression of the sucrose/proton symporter cscB [Ducat et al., Appl Environ Microbiol. 78: 2660-82 (2012)]. E. coli W was obtained from ATCC (accession no. 9637) and the E. coli W ΔcscR strain is described in Arifin et al. (Biotechnol. 156:275-8 (2011)). B. subtilis 168 was obtained from ATCC (accession no. 23857) and B. subtilis 3610 ΔsinI is described by Kearns et al. (Mol Microbiol. 55:739-49 (2005)). The ΔsinI mutant strain of 3610 was used to minimize chained growth making CFU counts of the strain reproducible (Kearns et al. Mol Microbiol. 55:739-49 (2005)). S. cerevisiae strains, WT W303 and W303^{Clump} (previously referred to as Ancestor and Recreated02 strains, respectively) are described by Koschwanez et al. (Elife. 2013:1-27 (2013)). All strains are listed in Table 1.

TABLE 1
Strains

| Strain | Origin |
|---|---|
| Synechococcus elongatus PCC7942 | ATCC 33912 |
| Synechococcus elongatus | Ducat et al., Appl Environ Microbiol. |
| trc-lac/cscB | 78: 2660-8 (2012) |
| Bacillus subtilis 168 | ATCC 23857 |
| Bacillus subtilis 3610 ΔsinI | Kearns et al., Mol Microbiol. |
| | 55: 739-49 (2005) |
| Escherichia coli K-12 BW25113 | Datsemkp et al. Proc Natl Acad Sci |
| | USA 97: 6640-5 (2000) |
| Escherichia coli W | ATCC 9637 |
| Escherichia coli W ΔcscR | Arifin et al. J Biotechnol. 156: |
| | 275-8 (2011) |
| Saccharomyces cerevisiae W303 | Ancestor strain; Koschwanez et al. |
| | Elife 2013: 1-27 (2013) |
| Saccharomyces cerevisiae | Recreated02 strain; Koschwanez et |
| W303^{Clump} | al. Elife 2013: 1-27 (2013) |
| Plasmid pAET41 | Peoples et al. J Biol Chem. 264: |
| | 15298-303 (1989) |

S. elongatus was propagated in BG-11 media (Sigma-Aldrich) plus 1 g/L HEPES, pH 8 in constant light at 35° C. In some cases S. elongatus engineered to express an IPTG-inducible sucrose symporter CscB (sometimes referred to S. elongatus CscB) was propagated in BG11 medium with 1 g/L HEPES, pH 8.3-NaOH. BG11 medium was prepared from 50× stock solution (Sigma-Aldrich, St. Louis, Mo.). M1 and M2 media were modifications of standard BG11 and were prepared from laboratory-created stock solutions (Table 2). Briefly, base BG11 medium was additionally supplemented with 15 mM NaNO₃, 4.5 mM K₂HPO₄ (phosphate buffering), 1.5 mM MgSO₄, 5 mM Na₂SO₄, 30 μM FeCl₃, 3 μM Na₂MoO₄, and 1× additional trace metals before the addition of KOH to pH 8.3. This mineral-enriched medium was designated “M1.” Second, a BG11 medium with reduced nitrate (only 2 mM NaNO₃), but an additional 4.5 mM K₂HPO₄ and 10 mM MgSO₄, was HCl corrected to pH 8.3. This nitrate-reduced medium was designated “M2.” Additional sodium chloride added to the three media recipes varied-depending on the intrinsic sodium content of each to maintain equivalent sodium contents.

TABLE 2
Media Composition

| Media Component (mg/L) | BG11 | M1 | M2 |
|---|---|---|---|
| Primary | | | |
| Sodium nitrate | 1500 | 2775 | 170 |
| Potassium phosphate dibasic | 40 | 823 | 823 |
| Magnesium sulfate•7H2O | 75 | 445 | 75 |
| Calcium chloride•2H₂O | 36 | 36 | 36 |
| Citric acid | 6 | 6 | 6 |
| Ferric ammonium citrate | 6 | 6 | 6 |
| EDTA disodium magnesium | 1 | 1 | 1 |
| Sodium carbonate | 20 | 20 | 20 |
| Trace | | | |
| Boric acid | 2.86 | 5.72 | 2.86 |
| Cobalt nitrate•6H₂O | 0.0494 | 0.0988 | 0.0494 |
| Cupric sulfate•5H₂O | 0.079 | 0.158 | 0.079 |
| Manganese chloride•4H₂O | 1.81 | 3.62 | 1.81 |
| Sodium molybdate•2H₂O | 0.39 | 8.0385 | 0.39 |
| Zinc sulfate•7H₂O | 0.222 | 0.444 | 0.222 |
| Additional | | | |
| HEPES | 1000 | | |
| Sodium sulfate | — | 710.2 | — |
| Ferric chloride | — | 4.866 | — |
| Magnesium chloride | — | — | 952.1 |
| pH 8.5 titration agent | NaOH | KOH | HCl |
| Calculated osmotic | 42.3 | 99.8 | 50.3 |
| concentration (Osm/L) | | | |

H. boliviensis was routinely cultured in liquid 1097 medium (ATCC BAA-759; (Quillaguamán et al., 2004)) and on 1.5% agar (BD Biosciences, San Jose, Calif.) 1097 medium plates for propagation and colony counts.

E. coli W strains deleted for the sucrose catabolism repressor cscR—which exhibit superior growth in co-culture with S. elongatus CscB (Hays et al., 2017)—were routinely cultured in LB medium and on 1.5% agar plates. The E. coli W strain was used to make chemically competent cells by the standard CaCl method, then transformed with the pAET41 plasmid (containing a pabABC expression cassette (Peoples et al., Journal of Biological Chemistry, 264(26): 15298-15303 (1989).

Co-cultures of CscB and H. boliviensis or E. coli were grown in the indicated media. Heterotrophic contamination was assessed using TSB agar plates as needed. All media elements were purchased from Sigma-Aldrich unless otherwise stated.

Axenic cyanobacteria were checked for contamination via plating on rich media. B. subtilis and E. coli were propagated in LB-Miller (EMD Millipore) while S. cerevisiae was maintained in YEPD media (MP Biomedicals). E. coli, B. subtilis, and S. cerevisiae were struck from frozen stocks on rich media plates (LB for bacteria and YEPD for yeast).

Co-culture media were optimized for either prokaryotes (^{CoB}BG-11) or S. cerevisiae (^{CoY}BG-11). ^{CoB}BG-11 consists of BG-11 supplemented with 106 mM NaCl, 4 mM NH₄Cl and 25 mM HEPPSO, pH 8.3-KOH. Indole (100 μM) was added to B. subtilis 168 co-cultures as indicated and in alpha-amylase experiments. ^{CoY}BG-11 consists of BG-11 supplemented with 0.36 g/L Yeast Nitrogen Base without amino acids (Sigma Aldrich), 106 mM NaCl, 25 mM HEPPSO, pH 8.3-KOH and 1 mM KPO3. Solid co-culture plates were composed of ^{CoB}BG-11 media with 1% autoclaved noble agar (BD Biosciences).

Where appropriate, media conditioned by S. elongatus was acquired by taking ^{CoB}BG-11 or ^{CoY}BG-11 and inoculating it with OD₇₅₀ 0.5 of cscB⁺ S. elongatus in baffled flasks, grown for 48 hours in constant light before filtration. Media conditioned by prokaryotic heterotrophs was made by inoculating ^{CoB}BG-11 supplemented with 0.2% sucrose with B. subtilis 3610 or W cscR E. coli at an OD₆₀₀ of 0.01 and allowing growth for 48 hours in a baffled flask before filtration.

For characterization of S. elongatus growth and sucrose production, S. elongatus was cultured axenically in baffled flasks of ^{CoB}BG-11 or ^{CoY}BG-11 and allowed to acclimate for at least 12 hours. Then cultures were adjusted to 25 mL with a final density of 0.5 OD₇₅₀. IPTG (1 mM) was added, as appropriate. This was the start of the experiment and is referred to as time 0. Cultures were monitored at 24-hour intervals by withdrawal of 1 mL culture. OD₇₅₀ was measured via photospectrometer (ThermoScientific NonoDrop 2000c) and culture supernatant was analyzed for sucrose content via a colorimetric Glucose-Sucrose Assay (Megazyme).

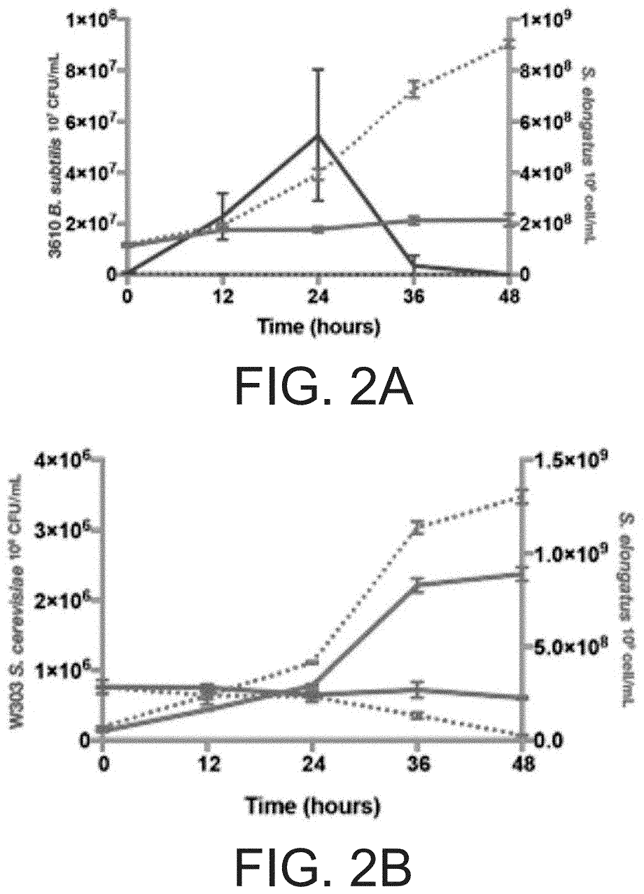

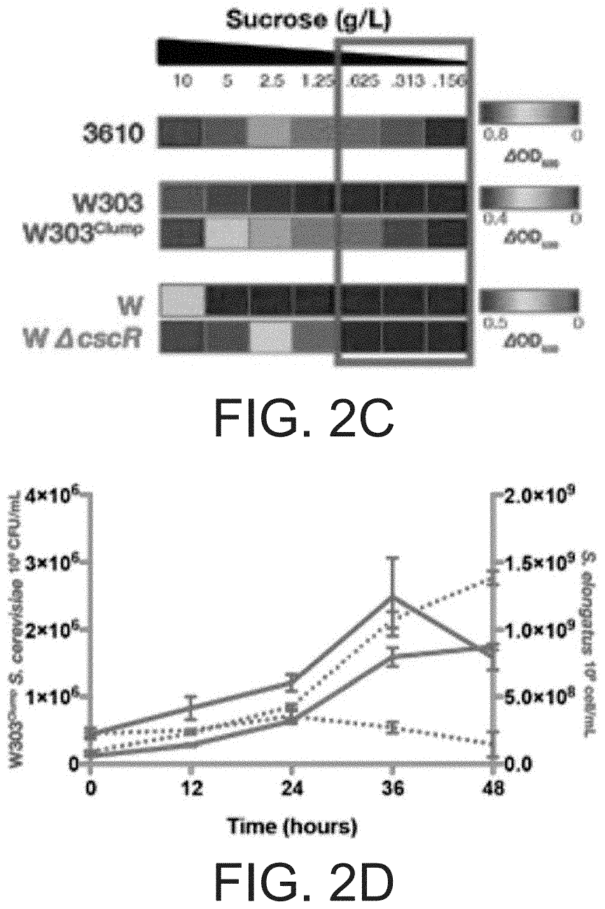

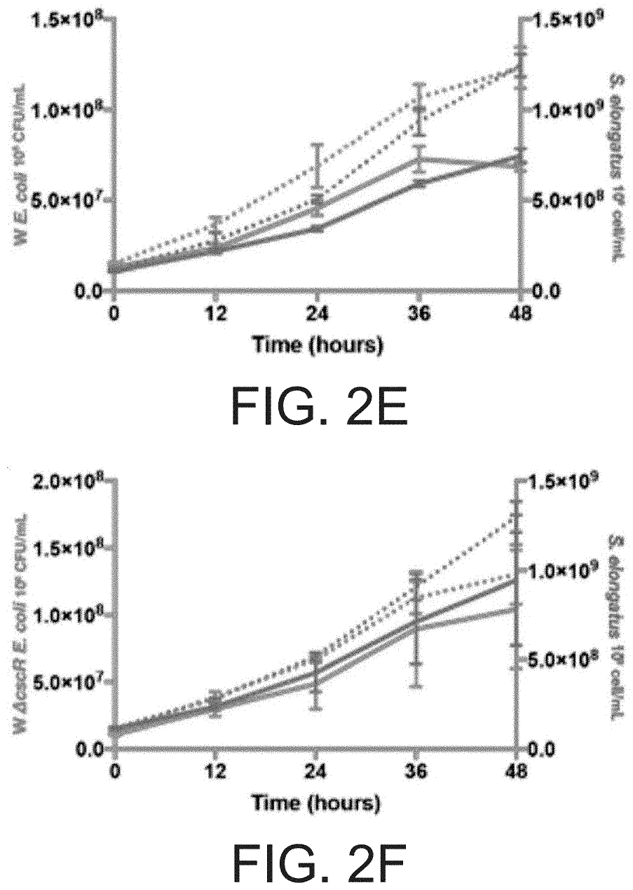

To prepare heterotrophic strains, single colonies were picked into their respective rich media and grown until turbid at varying temperatures before co-culture (37° C. for E. coli and B. subtilis; 30° C. for S. cerevisiae). Cells were diluted into the appropriate co-culture media +2% sucrose to acclimate to co-culture media and maintained within log phase growth (OD₆₀₀<0.70) before use in co-cultures. All acclimating cultures and co-cultures were grown at 35° C., 150 rpm, 2% CO2, in light (PAR=about 80 μmol m⁻² s⁻¹ with 15 W Gro-Lux Sylvania fluorescent bulbs) within a Multitron Infors HT incubator. Heterotrophic growth was measured by inoculating rinsed cells at 0.01 OD₆₀₀ (bacteria) or 0.05 OD₆₀₀ (yeast) into fresh co-culture media at the indicated sucrose concentration. Data for growth rate was collected from 25 mL flask cultures while 96-well plates with 1 mL culture volumes were used to assay growth in a gradient of sucrose concentrations (0.156 mg/mL to 10 mg/mL, FIG. 2C) as well as growth in conditioned media; OD₆₀₀ of plates were read on a BioTek Synergy Neo plate reader.

### Batch Co-Cultivation and Quantification

Flask co-cultures were completed in 25 mL volumes in baffled flasks. Cyanobacteria and heterotrophs were acclimated to ^{CoB}BG-11 or ^{CoY}BG-11 media prior to inoculation into co-cultures. All co-cultures were grown at 35° C., 150 rpm, 2% CO₂, in light (15 W; Gro-Lux; Sylvania) within a Multitron Infors HT incubator. 1 mM IPTG was added when indicated. Growth in co-cultures was monitored every 12 hours: S. elongatus was measured by the count of gated red-fluorescent events on a quantitative flow cytometer (BD Accuri); heterotrophs were assayed by plating dilution series on rich media to count colony forming units (CFU). Estimates of W303^{Clump} cell number were derived by counting CFUs, but numbers were adjusted for the 6.6 cells/clump, and as confirmed under our culture conditions. For dilution experiments, co-cultures containing E. coli or B. subtilis were grown for 24 hours before 10 or 100-fold dilutions.

In some cases, cultures and plates were grown at a constant 32-35° C. Some experiments were carried out in 125 mL baffled flasks in a Multitron HT Pro incubator (Infors, Bottmingen, Switzerland). Heterotrophs were grown in the dark on air with shaking at 250 rpm, while all phototrophic cultures and co-cultures were grown at 130 rpm, with 2% CO₂ and ˜80 μE m⁻² s⁻¹ PAR (15W Gro-Lux Sylvania bulbs). Media volumes were often 30 mL. S. elongatus CscB were routinely passaged by back-dilution to OD₇₅₀=0.3 and with 12.5 mg/L chloramphenicol. Chloramphenicol was not applied during co-culture experiments. CscB expression was induced with 1 mM IPTG (as previously reported (Ducat et al., 2012)) and also at greater than or equal to 12 h before experimentation or alginate encapsulation. H. boliviensis were maintained on 1097 plates and grown in 1097 liquid for 2 d before use in experiments.

S. elongatus CscB Alginate Encapsulation

3% sodium alginate (Sigma-Aldrich) was created by slow mixing over several hours, followed by vacuum degassing and autoclave sterilization. IPTG induced, S. elongatus CscB cells grown in BG11 medium for 24 h were harvested at OD₇₅₀≈1.5 by centrifugation and resuspended in 1/24 volume sulfur-free (no MgSO₄ added) BG11 medium. The resuspended cells were added 1:12 to 3% sodium alginate- and thoroughly mixed by gentle stirring to a final 2.75% sodium alginate content and roughly two-fold concentrated CscB cells (OD₇₅₀=3.0). In a sterile hood, this solution was then added dropwise to a ≥20-fold larger volume of 20 mM BaCl₂ using a vertically-oriented syringe pump (KD Scientific, Holliston, Mass.), 5 mL syringes (BD Biosciences), and 30G needles. The drops traveled ˜35 cm from needle to the slowly stirred BaCl₂ solution and cured at least 15 min in BaCl₂ before being twice rinsed with deionized water and further incubated in deionized water for 5 min. The beads were transferred into >2 μL of BG11 medium with 1 mM IPTG and indicated NaCl concentrations; the medium was exchanged at least 2× after >30 min incubations and the removal of excess residual barium was evaluated by precipitate formation after adding 1 M Na₂SO₄ to withdrawn supernatant. Equilibration was considered successful when residual barium no longer precipitated. Finally, beads were transferred into a baffled 4 μL Fernbach flask for 12 h, with the intended final medium and salt under constant light. Beads intended for use in M2 medium experiments were transferred into BG11 containing no nitrate (BG11-N) at a volume appropriate to diluting the 17.6 mM nitrate contained within the BG11 infused beads to the desired 2 mM. The completed beads were then apportioned into experimental flasks with fresh medium to begin experiments.

### Heterotroph Exposure to Variable Cyanobacteria Densities

B. subtilis and E. coli were recovered from rich media as above, washed in ^{CoB}BG-11 and inoculated at an OD₆₀₀ of 0.01 in ^{CoB}BG-11 media+2% sucrose with cyanobacteria at different densities (OD₇₅₀ at 0, 0.5, 1, and 2). S. cerevisiae was treated identically except they were inoculated at about 3×10⁵ cells/mL (OD₇₅₀=0.03) and ^{CoY}BG-1 was used. These samples were split into two 36-well plates and incubated and exposed to either constant light or dark conditions while maintaining the other growth parameters. Additional cultures of B. subtilis strain 3610 were set up as described above before addition of DCMU (3-(3,4-dichlorophenyl)-1,1-dimethylurea) in ethanol and (thiosulfate) in water to final concentrations of 40 μM and 190 mM, respectively. Vehicle was added to control cultures and further cultures were split and sealed with septa. One was kept with atmospheric gas, while the other was sparged for 5 minutes with gas devoid of oxygen (12:10:82 H₂:CO₂:N₂). Heterotroph counts were determined by plating on rich media for colony counts as above after initial setup (time 0) and after 12 hours of culture. Ratios of the viable cell counts from the light vs. dark cultures or log 10 of these ratios after 12 hours are reported.

### Structured Growth Perturbation

To test the ability of co-cultures to withstand environmental perturbation, flask co-cultures were inoculated and grown as previously described for 24 hours before plating of 100 μL on solid co-culture Petri dishes. After five days, uneven lawns of heterotrophs and cyanobacteria arose. Cells were picked from these plates into 96-well plates and allowed to grow for 2-5 additional days. Any well that demonstrated cyanobacterial growth (as judged visually by green appearance) at the end of 48 hours was spotted on rich media to determine the presence or absence of heterotrophic symbionts. Solid culture and 96-well plate growth was completed at 35° C., 0 rpm, 2% CO₂, in constant light (15 W; Gro-Lux; Sylvania) within a Multitron Infors HT incubator.

### Heterotroph Spotting on Cyanobacterial Lawns

Lawns of cscB⁺ cyanobacteria were achieved via spreading of 250 μL of cscB⁺ cyanobacteria (OD₇₅₀ 0.5) on solid co-culture plates with or without 1 mM IPTG. After the cyanobacteria had absorbed on to the plate (more than 3 hours in the dark), 3 μL drops of heterotrophs were spotted on to the lawns. Heterotrophs had been previously grown up in rich media and washed three times to remove any media components before spotting. Media blanks and boiled cells were spotted as negative controls. Plates were then grown at 35 C, 2% CO₂,in constant light (15 W; Gro-Lux; Sylvania) within a Multitron Infors HT incubator.

### Long-Term Continuous Co-Cultivation

Long-term co-cultures were incubated in Phenometrics Environmental Photo-Bioreactors with 150 mL liquid volumes of a mix of cscB⁺ S. elongatus with either S. cerevisiae W303^{Clump} or E. coli W ΔcscR in the appropriate co-culture BG-11 media+1 mM IPTG. Reactors were seeded with about 1×10⁸ cells/mL of S. elongalus (OD₇₅₀=0.5) and a final concentration of heterotroph equivalent to about 1×10⁶ cells/mL S. cerevisiae W303^{Clump} (final OD₆₀₀ about 0.1) or about 5×10⁷ cells/mL E. coli W ΔcscR (OD₆₀₀˜0.05). Light was provided by onboard white, high-power LEDs (400 μmol m⁻² s⁻¹) continuously for E. coli W ΔcscR cultures. and with a 16:8 light:dark photoperiod for S. cerevisiae W303^{Clump} co-cultures. The total density of co-cultures was monitored by on-board infrared diodes, following a brief (3-12 hour) acclimation period where the time-averaged optical density was allowed to settle to a fixed point following culture initiation. This measurement was used to control attached peristaltic pumps that eject fresh media to maintain the set target OD as previously described [Lucker et al. Algal Res. 6(PB):242-9 (2014)]. Co-culture temperature was maintained at 30° C. by a heated jacket; cells were agitated continuously by a magnetic stirbar. Daily, about 2 mL of co-culture volume was withdrawn and cyanobacterial and heterotrophic cell counts determined by flow cytometry and plating, respectively (as described above).

### Alpha-Amylase Production and Quantification

For the production of alpha-amylase, co-cultures of cscB S. elongatus and B. subtilis strain 168 were completed in 8 mL volumes of ^{CoB}G-₁₁ supplemented with 100 μM indole in 6 well dishes. When specified, cyanobacteria were present (OD₇₅₀=0.5) with or without 1 mM IPTIG. Control cultures did not contain cyanobacteria. Alpha-amylase production was measured after 24 hours of culture at 35° C., 0 rpm, 2% CO₂, in constant light (15 W; Gro-Lux; Sylvania) within a Multitron Infors HT incubator. Alpha-amylase activity in supernatants was measured immediately after pelleting of cultures with the EnzChek Ultra Amylase Assay Kit, Molecular Probes Life Technologies using the manufacturer's protocol. Western blots confirmed presence of alpha-amylase in supernatants after addition of NuPAGE LDS sample buffer (Invitrogen) followed by 10 minutes at 100° C. Protein (10 L) was run on NuPage 4-12% Bis-Tris gels (Life Technologies) for in MES SDS running buffer for 50 minutes at 185 V. The iBlot 2 Dry Blot System (ThermoScientific) was used to transfer protein to nitrocellulose membranes (iBlot 2 NC Regular Transfer Stacks). Anti-alpha amylase antibodies (polyclonal rabbit; LS-C147316; LifeSpan BioSciences; 1:3,000 dilution) were used as the primary antibody followed by peroxidase-conjugated donkey anti-rabbit antibodies (AffiniPure 711-035-152 lot 92319; Jackson ImmunoResearch; 1:5,000 dilution) as the secondary antibody. The western blot was visualized via Western Lightning Plus-ECL, Enhanced Chemiluminescence Substrate (PerkinElmer, ProteinSimple FluorChem M). Purified alpha-amylase (Sigma Aldrich) was used as a control in all assays.

### Polyhydroxybutyrate (PHB) Production & Quantification

E. coli strains were transformed with pAET41 (Table 1) before use in co-cultures for production [Peoples et al. J Biol Chem. 264:15298-303 (1989)]. Co-cultures were set up as previously described in 25 mL flasks. After one week of growth, the entire culture was spun down, frozen, and stored at −80° C. until PHB content was quantified. PHB content was quantified by methods described by Torella et al. (Proc Natl Acad Sci. 112:2337-42 (2015)) and Lui et al. (Nano Lett 15(5):3634-9 (2015)). Briefly: cell pellets were digested with concentrated H₂SO₄ at 90° C. for 60 min. The digestion solution was diluted 500 times (500×) with water and passed through 0.2 μm filter. The solutions were subsequently analyzed by a high-performance liquid chromatography (HPLC, Agilent HPLC 1200) equipped with Aminex HPX-87H column and UV absorption detector. The volume of each sample injection was 100 PL. The mobile phase was 2.5 mM H₂SO₄ aqueous solution, with a flow rate of 0.5 mL/min for 60 min. 5 mM sodium acetate (Sigma Aldrich) was added as an internal standard. The concentrations of PHB were determined by comparing the peak area with that in standard curves from 0.1 to 30 mM.

### Light Microscopy

Cells were imaged on agarose pads using an inverted Observer D1 Microscope (Zeiss, Jena, Germany) with a Plan-Neofluar 100× oil objective (Zeiss), AxioCam ICC5 camera (Zeiss), X-Cite Series 120 mercury halide bulb (Lumen Dynamics, Mississauga, Canada), and using ZEN 2012 software (Zeiss).

### Transmission Electron Microscopy

Alginate encapsulated S. elongatus were fixed for 48 h at 4° C. in 2.5% paraformaldehyde/glutaraldehyde in 0.1 M sodium cacodylate buffer (pH 7.4), while H. boliviensis cells were fixed for 24 h. All samples were washed three times with 0.1 M sodium cacodylate buffer and subsequently fixed in 1% osmium tetroxide/1.6% potassium ferricyanide solution in 0.1 M sodium cacodylate buffer utilizing a MS-9000 Laboratory Microwave Oven (Electron Microscopy Sciences, Hatfield, Pa.; 5 min at 30 C). Samples were washed with deionized water until clear from osmium and post-fixed with 1% aqueous uranyl acetate for 2 min at 30° C. Samples were washed with deionized water three times more and subsequently dehydrated with an increasing acetone series (2 min at 30° C.) and then embedded in Spurr's resin (20 min, 30° C., 25% increments). Blocks were left to polymerize at 60° C. for 3 d. Sections (45-nm thick) were cut on a MX ultramicrotome (RMC Boeckeler, Tucson, Ariz.). Prior to imaging on a JEM 100CX II transmission electron microscope (JEOL) equipped with a Prius SC200-830 CCD camera (Gatan, Pleasanton, Calif.), sections were stained with 1% uranyl acetate and Reynolds lead citrate (Reynolds, 1963) for 5 min each.

### Quantification

Culture optical densities were measured using >500 μL of culture in a cuvette with a Genesys 20 (Thermo Fisher Scientific, Waltham, Mass.) spectrophotometer. H. boliviensis were typically measured at OD₆₀₀, S. elongatus CscB at OD₇₅₀, and both for co-culture experiments, unless otherwise stated. Chlorophyll a was similarly measured at OD₆₆₅ following extraction of an appropriate cell volume within 1 mL 95% methanol (Zavirel et al., 2015).

H. boliviensis or E. coli cell densities were quantified by 96-well plate serial dilution in BG11+175 mM NaCl medium and plating on agar solidified 1097 plates or LB plates, respectively. Colony forming unit (CFU) counts were made after 36 h of incubation at 30° C. for H. boliviensis, or overnight at 37° C. for E. coli. Heterotrophic contamination was routinely assessed from the same serial dilutions by spotting onto TSB plates.

Sucrose was measured using a Sucrose/D-Glucose Assay Kit (Megazyme, Bray, Ireland). Briefly, 200 μL of cell culture was pipetted into a 96-well plate, centrifuged at 3 krpm for 30 min at 4° C. (Sorvall 75006445; New Castle, Del.), and 100 μL of cell-free medium withdrawn. To assay sucrose, 20 μL of cell-free medium was mixed with 10 μL invertase solution, sealed, and incubated at 50° C. for >20 min, then incubated with 150 μL GOPOD reagent solution at 50° C. for 20 min. Absorbance at 510 nm after cooling was measured on a microplate reader (SpectraMax M2; Molecular Devices, Sunnyvale, Calif.). Sucrose concentrations were determined in comparison to a six-point standard curve containing 0 to 0.25 mg/mL glucose samples, which typically exhibited a R²≥0.98 linear fit.

Polyhydroxybutyrate (PHB) was measured using the crotonic acid method and high-performance liquid chromatography (HPLC; (Van-Thuoc et al., 2008)). Briefly, cells were centrifuged at 3,700×g for 30 min, decanted, and lyophilized before weighing of the resultant dry cell pellets. In a sealed glass vial, the dry cell pellets were dissolved in 1 mL of concentrated sulfuric acid, heated to 90° C. for 1 h, cooled to room temperature, and then diluted 100-fold with deionized water. The resulting solutions were filtered with GHP membranes (Pall Life Sciences, Port Washington, N.Y.) to remove particulates and 20 μL injected onto an Aminex 300-mm HPX-87H (Bio-Rad Laboratories, Hercules, Calif.) column. The mobile phase was 0.028 N H₂SO₄ and flowed at 1 mL/min, while the column was maintained at 60° C. UV-absorption was monitored at 210 nm. Two standards of commercial polyhydroxybutyric acid (Sigma-Aldrich) were similarly treated, injected at 6 volumes at the start and end of sampling each, and averaged to create a standard curve with R²=1 linear fit.

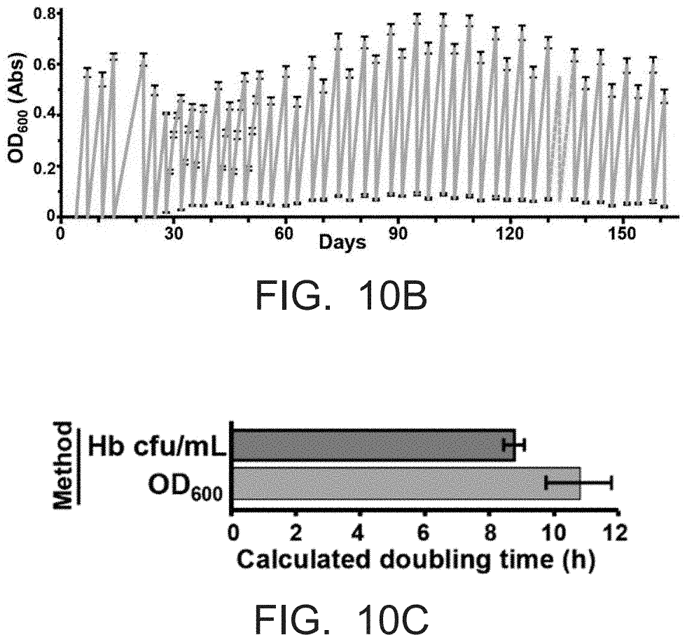

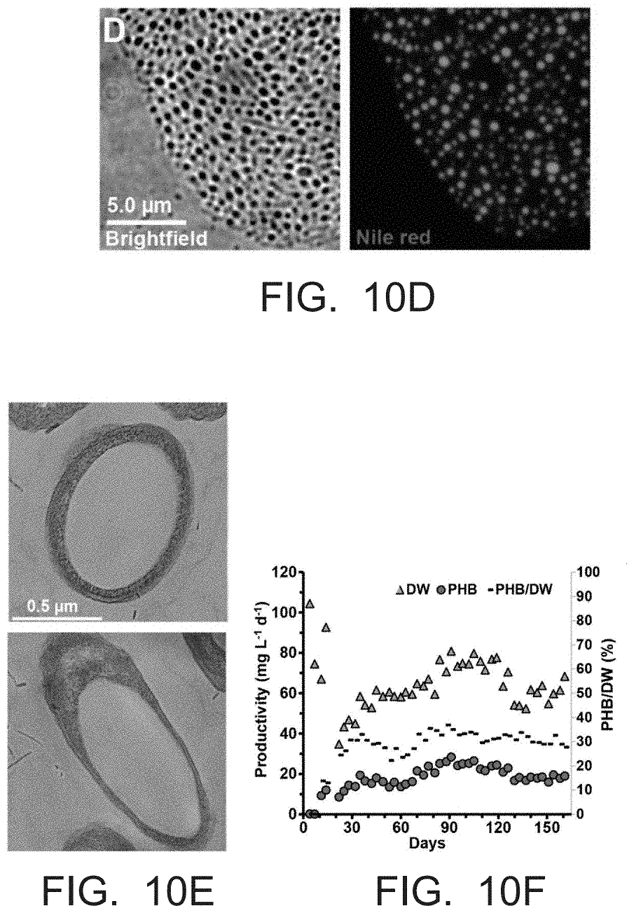

Free nitrate and nitrite were estimated by AquaChek (Hach) test strips during co-culture of S. elongatus CscB and H. boliviensis. This colorimetric assay is sensitive to free nitrate and nitrite levels ≥2 ppm (≥32 μM). In the nitrogen limited experiments described in FIG. 10, free nitrate levels dropped from 2 mM (the base levels of M3 media; Table 2) to below the detection limits within 24 hours following each back-dilution.

Contaminant 16s rRNA Sequencing

Contaminating bacterial colonies isolated from co-culture were grown for use in 16s rRNA PCR sequencing reactions with universal primers 8F, 27F, 1100R, and 1492R, as previously described (Lane, 1991; Turner et al., 1999). Sequence BLAST searches (https://blast.ncbi.nlm.nih.gov/Blast.cgi) returned only >97% identity and e-values >7e⁻¹²⁶ results with Stenotrophomonas maltophilia the most numerous and consistently identified of named species.

### Mathematical Framework, Statistics, and Figures

All equations were modeled in Mathematica (Wolfram Research, Inc., Mathematica, Version 11.0). Statistics were completed in GraphPad Prism version 7 GraphPad Software. La Jolla Calif. USA (see website at graphpad.com).

### Example 2: Cyanobacteria can Form Consortia with Heterotrophs

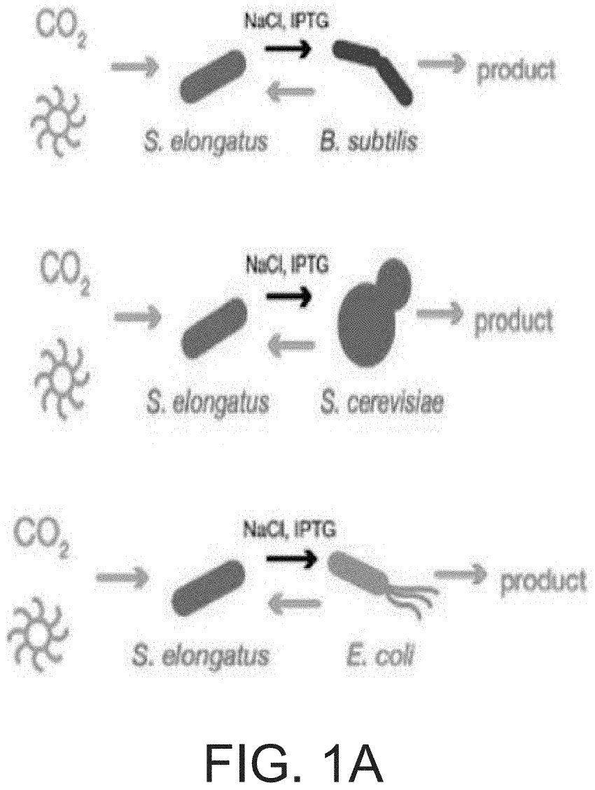

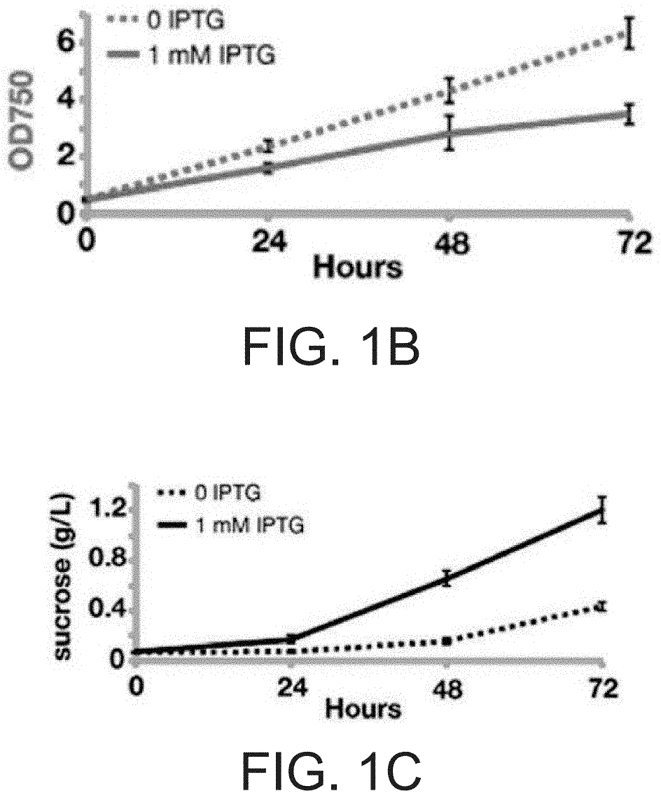

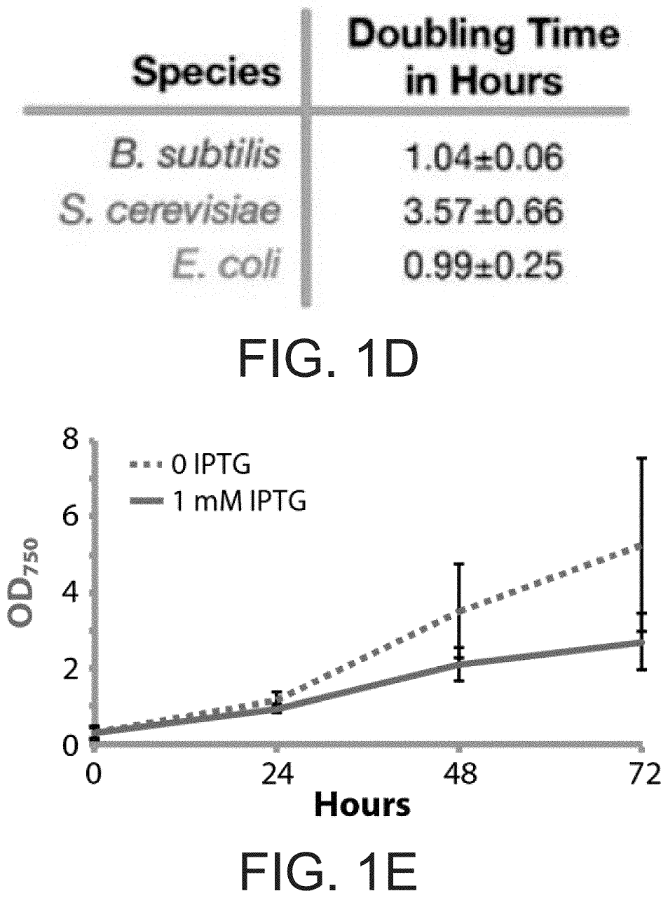

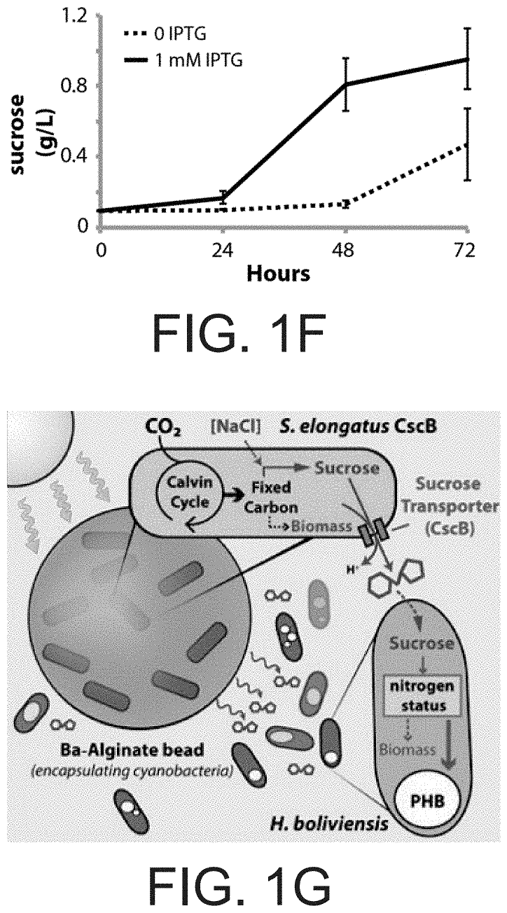

Pairwise consortia were designed where cscB* S. elongatus secreted sucrose in response to osmotic pressure combined with induction of cscB expression by isopropyl β-D-1-thiogalactopyranoside (IPTG) [Ducat et al. Appl Environ Microbiol. 78: 2660-8 (2012)]. Carbon secreted by cyanobacteria promoted growth of co-cultured heterotrophs as schematically illustrated in FIG. 1A.

Media with optimized compositions of nitrogen, salt, and buffer were developed: termed ^{CoB}BG-11 for use in cyanobacteria/bacteria consortia and ^{CoY}BG-11 for cyanobacteria/yeast co-culture (see Example 1). Tests verified that S. elongatus grows and produces sucrose in both ^{CoB}BG-11 and ^{CoY}BG-11 (FIGS. 1B, 1C, 1E, 1F). Induction of cscB greatly enhanced the rate of sucrose export, and this redirection of carbon resources leads to slower growth of cscB* S. elongatus (FIGS. 1B, 1C, 1E, 1F). All heterotrophs were assessed to confirm that they were capable of growth in axenic monocultures in these defined media when provided with exogenous sucrose (2%) as the sole carbon source (FIG. 1D).

CscB⁺ S. elongatus directly supported heterotroph growth in co-cultures that contain no external carbon sources (FIG. 2A-2F). In all consortia, cscB+ S. elongatus was inoculated with a heterotrophic microbe in the appropriate co-culture media (with or without the addition of 1 mM IPTG to induce cscB expression; see Example 1) and grown over 48 hours in constant light. The growth of cscB⁺S. elongatus was tracked in co-culture via flow cytometry. Viable heterotrophs were tracked by analyzing the number of colony forming units (CFUs) when plated on the appropriate solid media. More than one strain of E. coli and S. cerevisiae were analyzed in co-culture to determine the effects of particular genetic backgrounds on growth kinetics.

B. subtilis growth in co-culture is dependent on IPTG-induced sucrose secretion from cscB⁺ S. elongatus (FIG. 2A). Without induction of cscB to enable sucrose secretion, B. subtilis fails to grow, indicating that sucrose availability is limiting at basal levels of sucrose export. However, when IPTG is added to increase sucrose export, B. subtilis growth is nonmonotonic: after an initial viability increases, viability decreases over the next 24 hours of co-culture (FIG. 2A).

S. cerevisiae growth in co-culture is dependent on genetic engineering to improve sucrose utilization. Wild type (WT) S. cerevisiae W303 did not grow in co-culture with or without IPTG induction (FIG. 2B). The capacity of WT S. cerevisiae W303 to grow axenically at low sucrose concentrations was examined. Poor/no growth was observed below 2.5 g/L sucrose (FIG. 2C), a higher concentration of sucrose than is produced by cscB⁺ S. elongatus at 48 hours (FIG. 1C).

The engineered strain, referred to as W303^{Clump}, from directed evolution experiments of S. cerevisiae W303 in low sucrose media [Koschwanez et al. Elife 2013:1-2725 (2013)]. W303^{Clump} (originally called Recreated02 in Koschwanez et al. 2013) contains mutations in genes CSE2, IRA1, MTH1, and UBR1 that enhance fitness in dilute sucrose, and also contains a nonsense mutation in ACE2 that compromises the full septation of budding daughter cells from the mother, resulting in small clonal cell aggregates (about 6.6 cells per clump on average). These aggregates grow in low sucrose due to increased local cell concentration and increased hexose availability after extracellular cleavage of sucrose by an invertase [Koschwanez et al., PLoS Biol. 9(8):e1001122 (2011)]. Unlike the parental strain, axenic cultures of W303^{Clump} exhibited some growth at all tested sucrose concentrations greater than or equal to 0.156 g/L (FIG. 2C), as well as when co-cultured with IPTG-induced cscB⁺ S. elongatus (FIG. 2D). Similar to B. subtilis, in co-culture W303^{Clump} S. cerevisiae demonstrate declining viability after an initial period of growth (FIG. 2D).

E. coli W grows in co-culture independently of induced sucrose secretion from cscB+ S. elongatus (FIG. 2E). In axenic culture, wild type (WT) E. coli W exhibits growth only when supplemented with greater than 5 g/L sucrose (FIG. 2C), well above the levels cscB+ S. elongalus secrete during 48 hours of growth (FIG. 1C). The growth of an E. coli W strain engineered for growth on sucrose was therefore tested. The sucrose catabolism repressor, cscR, was deleted within the E. coli W strain, and this was hereafter referred to as ΔcscR E. coli). The ΔcscR E. coli strain exhibits more rapid growth at lower sucrose concentrations [Archer et al., BMC Genornics 12:9. 27-29 (2011); Sabri et al. Appl Environ Microbiol. 79:478-87 (2013); Arifin et al. J. Biotechnol. 156:275-8 (2011)].

Monocultures of ΔcscR E. coli exhibit the capacity to grow on lower concentrations of sucrose (as low as 1.25 g sucrose/L; FIG. 2C), yet they still have a relatively low capacity to utilize dilute sucrose comparison to S. cerevisiae and B. subtilis (FIG. 2C), and demonstrate no growth at sucrose concentrations in the range that cscB+ S. elongatus can secrete in 48 hours (FIG. 1C, green box FIG. 2C). In co-culture, ΔcscR E. coli exhibited the same monotonic growth pattern as the unmodified strain (FIG. 2E-2F).

These data indicate that in the first days of co-culture, while exported sucrose concentrations are low (less than or equal to I g/L), E. coli strains cannot utilize sucrose effectively and dominantly depend on other metabolites from S. elongatus; perhaps extracellular polymeric substances.

### Example 3: Light Driven Cyanobacterial Metabolism Inhibits Heterotroph Viability

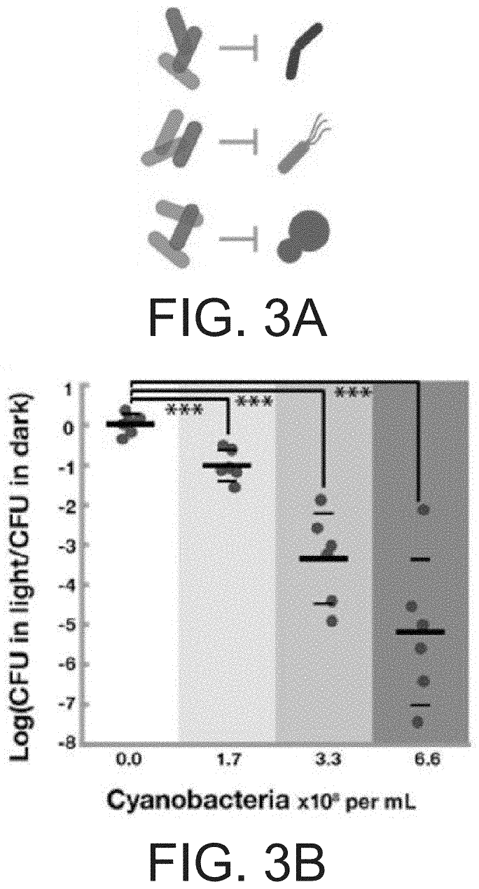

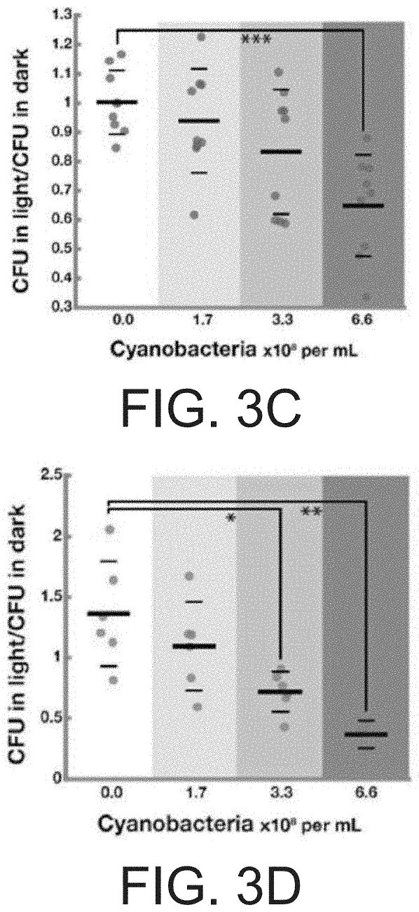

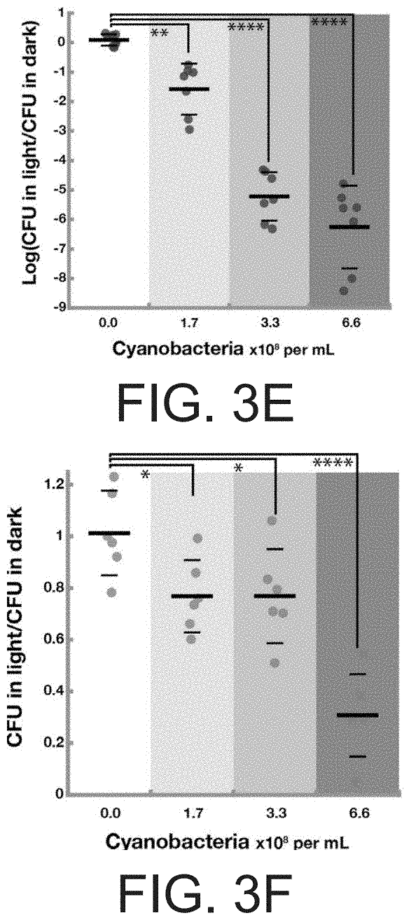

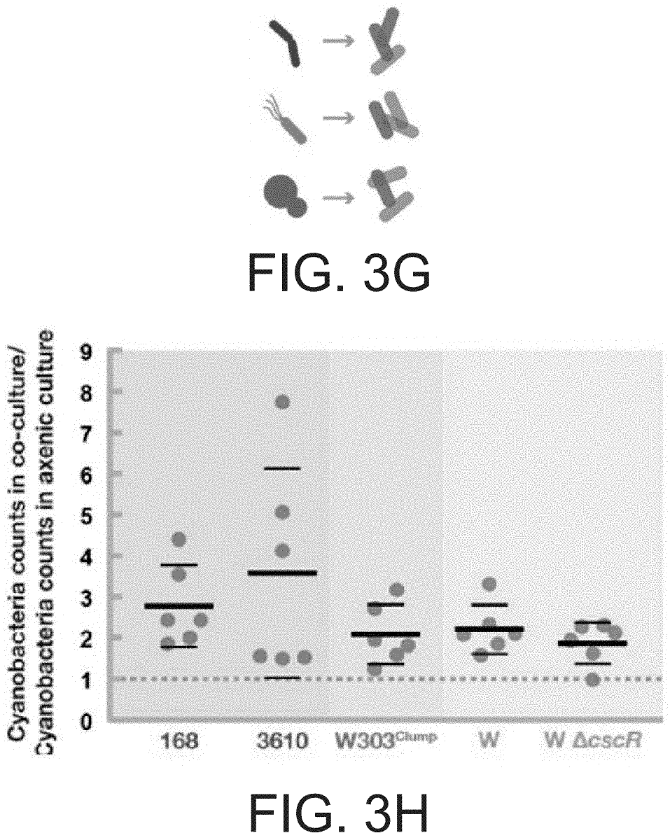

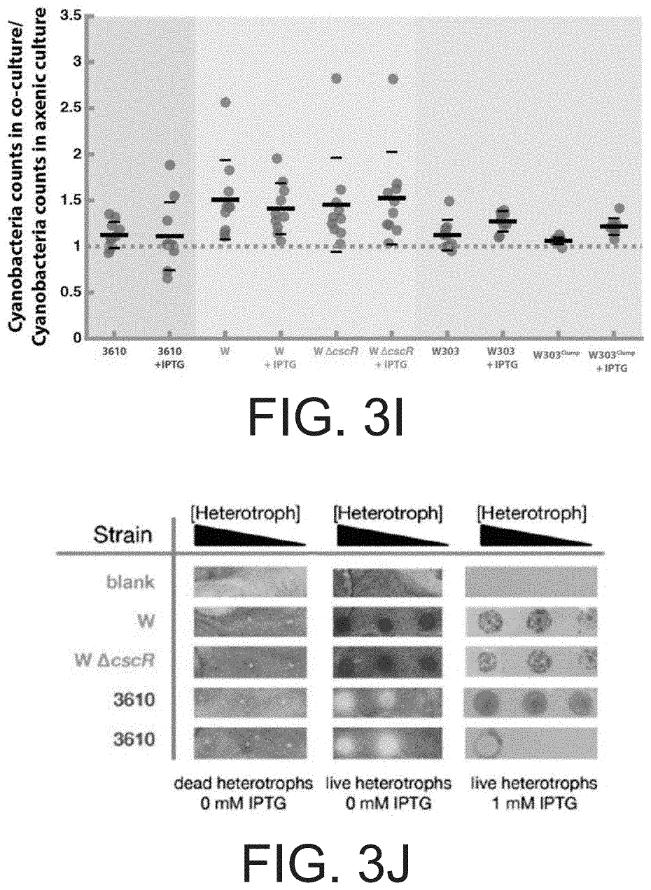

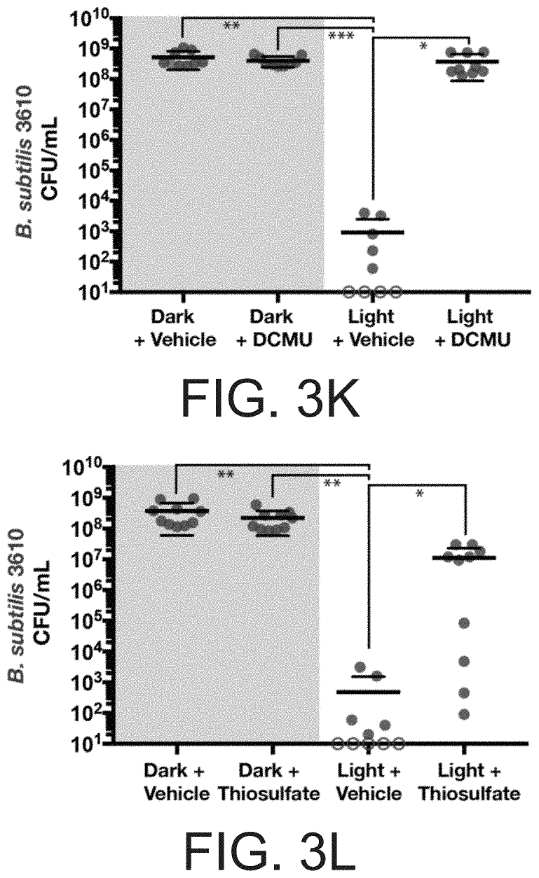

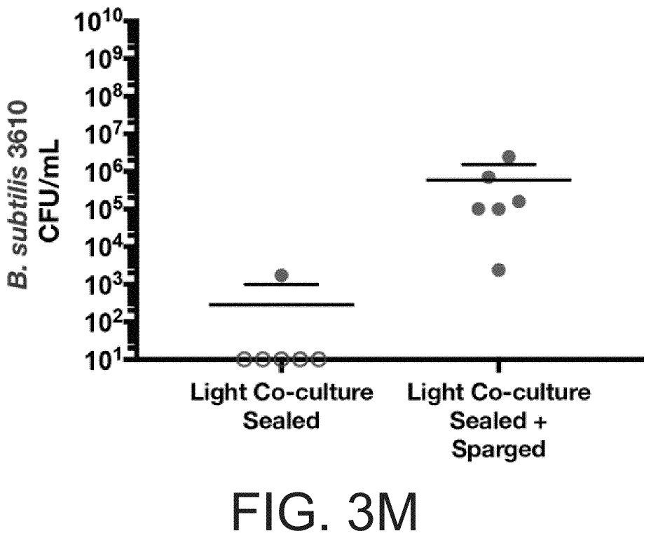

As illustrated in FIG. 3A, cyanobacterial light driven metabolism is a source of heterotroph growth inhibition when sucrose is not limiting. The lack of monotonic growth in B. subtilis and S. cerevisiae co-cultures indicates that interactions beyond sucrose feeding are occurring between heterotrophs and cyanobacteria (FIGS. 2A and 2D).

Experiments were performed to determine the source of the heterotroph growth inhibition. To focus on products other than sucrose concentrations that could influence heterotrophic viability and eliminate the confounding factor that cyanobacteria only generate sucrose in the light [Ducat et al., Appl Environ Microbiol. 78: 2660-8 (2012)], co-cultures were supplemented with exogenous sucrose (2%) and cultivated in the light or dark. After 12 hours of co-cultivation, heterotroph viability of each of the three species (S. elongatus, B. subtilis and S. cerevisiae) was determined.

FIG. 3B graphically illustrates growth of B. subtilis 3610 co-cultured with various concentrations of S. elongatus. FIG. 3C graphically illustrates growth of W303Clump S. cerevisiae co-cultured with various concentrations of S. elongatus. FIG. 3D graphically illustrates growth of E. coli W ΔcscR co-cultured with various concentrations of S. elongatus. FIG. 3E graphically illustrates that additional heterotrophic prokaryotes, including B. subtilis production strain 168, demonstrates sensitivity to S. elongatus in the light. FIG. 3F graphically illustrates that additional heterotrophic prokaryotes, including E. coli strain W, exhibit sensitivity to S. elongatus in the light.

FIG. 3B-3F illustrate that decreased growth or death of heterotrophs correlated with increasing concentrations of cyanobacteria solely in illuminated cultures. This effect is most apparent in strains of B. subtilis (FIG. 3B, 3E) where the viability of heterotrophic species decreased by orders of magnitude when co-cultured in the light with high concentrations of S. elongatus.

However, the inhibition of B. subtilis growth while in co-culture with S. elongalus was mitigated when cells were incubated with DCMU, an inhibitor of oxygen evolution from Photosystem 11, or thiosulfate, a potent antioxidant (FIG. 3K-3M). Likewise, B. subtilis also persists in the presence of dense S. elongatus when oxygen is sparged (12:10:82 H₂:CO₂:N₂) from the headspace of co-cultures (FIG. 3M).

These data indicate that the factor that inhibited heterotroph growth when high concentrations of cyanobacteria were present, was oxygen. Oxygen levels can readily be regulated (e.g., reduced) by sparging with low oxygen-containing gases or addition of antioxidants, as illustrated in FIG. 3K-3M.

### Example 4: Heterotrophic Species Stimulate Cyanobacterial Growth

Unlike the effects on heterotroph viability, co-culture with heterotrophs can stimulate growth of S. elongatus (FIG. 3G, 3I). As illustrated in FIG. 3I batch-culture cyanobacteria counts were higher in co-cultures then in control monocultures of cyanobacteria at various time points, Because batch cultures of relatively dense S. elongatus can negatively impact heterotrophic viability (FIG. 2A, 2D; FIG. 3A-3D), and also lead to significant self-shading, low concentrations of cscB⁺ S. elongatus induced with IPTG were inoculated into co-cultures with heterotrophs. After 48 hours of co-culture, cyanobacteria numbers in co-culture were normalized to axenic cyanobacteria controls. Significant increases in cyanobacterial growth were observed in the presence of heterotrophic microbes, with total cyanobacterial cell counts increasing by between 80 and 250% on average (FIG. 3H).

The growth-promoting effect of heterotrophs on cyanobacteria is also observed when co-cultivated on solid media. Dilutions of B. subtilis or E. coli cultures were spotted on a lawn of dilute cyanobacteria with or without IPTG (FIG. 3J). Areas of the cyanobacterial lawn overlaid with E. coli exhibited more rapid growth than the surrounding lawn of S. elongatus alone.

The growth-promoting effect of B. subtilis on cscB⁺ S. elongatus was dependent upon induction of sucrose export. Without IPTG, spots of B. subtilis inhibited cyanobacterial growth (FIG. 3J). However, in the presence of IPTG, B. subtilis stimulated cyanobacterial growth. Cyanobacterial colonies emerged either throughout. or at the periphery of the spot where B. subtilis strain 3610 was plated (FIG. 3J; “3610” top and bottom panels, respectively). S. cerevisiae was not assayed in this manner, because of poor growth of cscB⁺ S. elongatus on ^{CoY}BG-11 solid agar plates.

Collectively, these experiments indicate that all three evolutionarily unrelated heterotrophs can significantly increase cyanobacterial growth under a range of growth conditions.

### Example 5: Robustness in Designed Photosynthetic Consortia

The inhibitory effects of cyanobacteria on the viability of heterotrophs was only observed at relatively high density and in the light (FIG. 3A-3D). Co-cultivation strategies were evaluated as described in this Example that would prevent over-growth of cyanobacteria to determine if heterotrophic viability could be maintained in the long-term.

Phenometrics environmental Photo-bioreactors (ePBRs) were first employed. Such ePBRs have turbidostat capabilities in addition to control of light, temperature, and culture stirring [Lucker et al. Algal Res. 6(PB):242-9 (2014)].

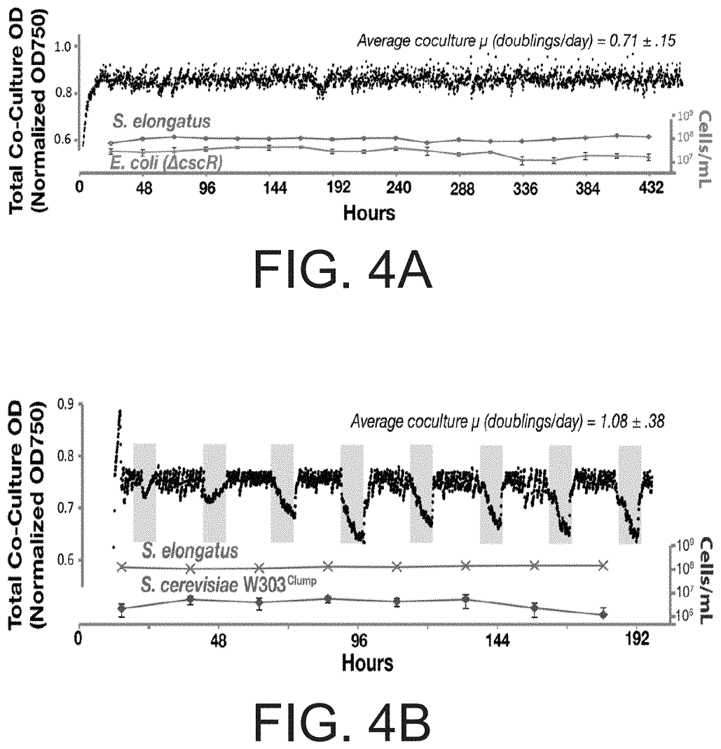

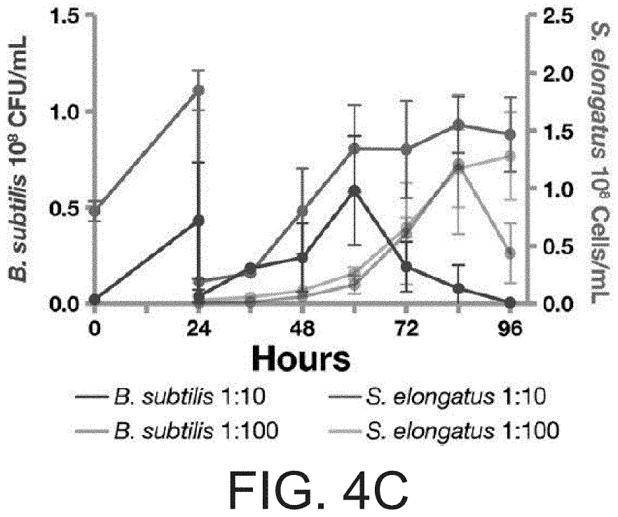

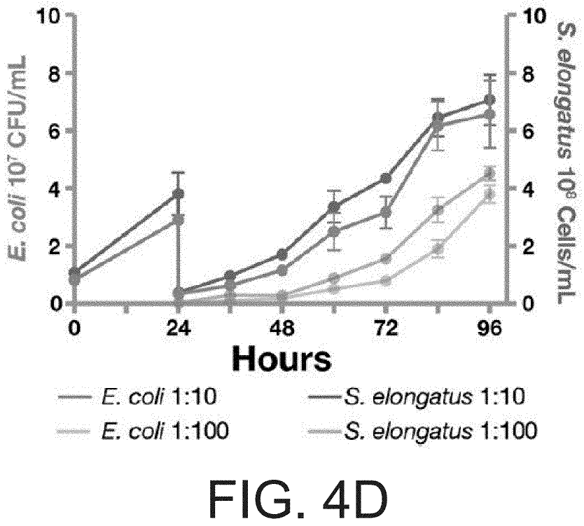

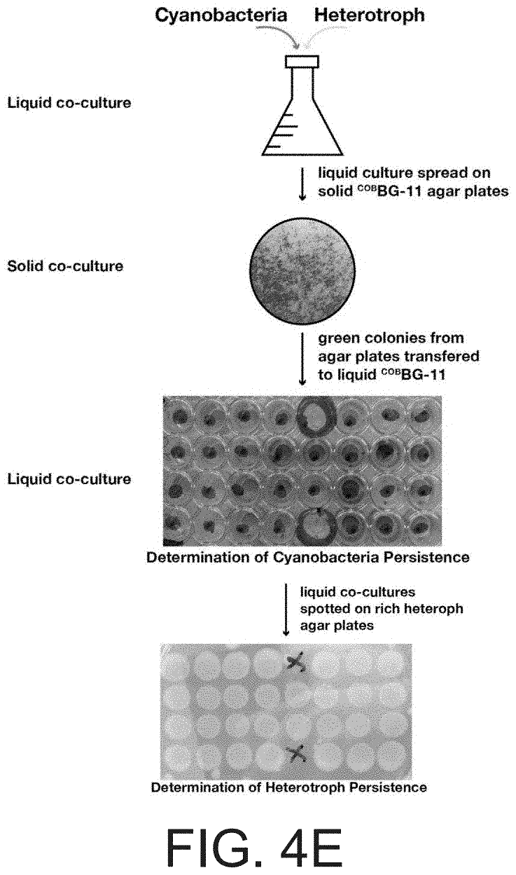

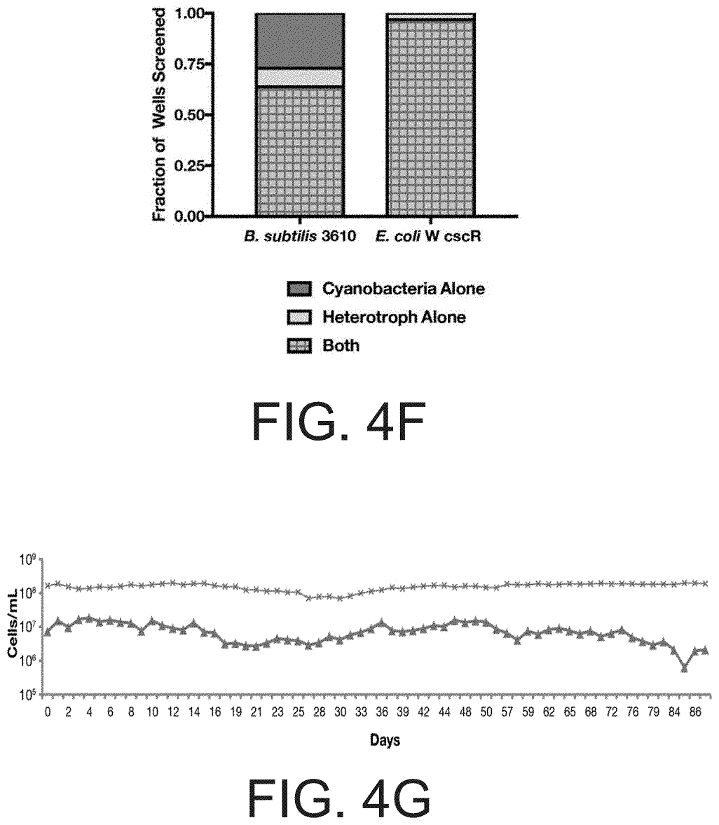

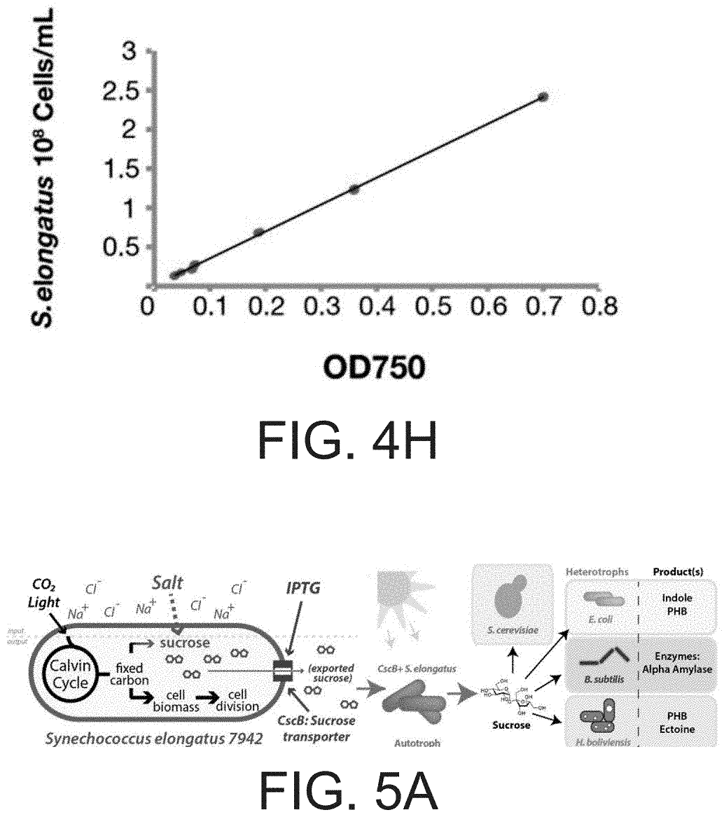

When cultivated at a constant density, E. coli/cscB⁺ S. elongatus co-cultures persist over time and heterotrophic viability is maintained. Co-cultures of induced cscB⁺ S. elongatus and strain W ΔcsR E. coli were grown continuously in ePBRs under constant light (FIG. 4A). Cell counts of cscB⁺ S. elongatus and CFUs of E. coli CFUs were monitored. As shown in FIG. 4A, the co-cultures maintained stable ratios of these organisms for more than two weeks.

Similarly, when cultured in ePBRs, S. cerevisiae W303^{Clump} maintained viability in co-cultures with cscB⁺ S. elongatus over time and also persisted through variable light conditions. The cscB⁺ S. elongatus was induced to secrete sucrose and inoculated with S. cerevisiae into culture media containing ePBRs. To examine the co-culture capacity to persist through light perturbations, these cultures were programmed with an alternating diurnal illumination regime (16 hours light: 8 hours dark, FIG. 4B). Natural growth environments would inevitably experience changes in light quantity/quality. Sustained growth in these continuous cultures indicates that yeast persist through periods of darkness when cyanobacteria are unable to supply sucrose or other photosynthates (FIG. 4D). In similar experiments that were extended over longer time periods, S. cerevisiae W303^{Clump} maintained viability in continuous culture with sucrose-secreting S. elongatus for greater than two months (FIG. 4G-4H).

Prokaryotic co-cultures with cyanobacteria persist through population bottlenecks and changes in environmental structure, B. subtilis/cscB⁺ S. elongatus and W ΔcscR E. coli/cscB⁺ S. elongatus co-cultures were subjected to large dilutions (1 to 10 or 1 to 100) to determine viability following the introduction of a population bottleneck. Cyanobacterial growth was monitored via flow cytometry following dilution, while heterotroph growth was measured via CFU, In perturbed cultures, heterotrophs can return to pre-dilution levels within three days (FIGS. 4C-4D). The persistence of heterotrophs in co-culture was also examined following plating onto solid media after growth in liquid co-culture. Co-cultures containing cscB⁺ S. elongalus and B. subtilis 3610 or W ΔcscR E. coli were moved from liquid to solid environments and back again (FIG. 4E). This transfer is expected to disrupt the ratio of different species within co-culture and alters any interactions dependent on the co-culture constituents being in well-mixed environments. After incubating co-cultures in the light on agar plates that have no added carbon, green colonies from the agar plate were picked into liquid ^{CoB}BG-11. Picked co-cultures were recovered in constant light for 2-5 days; wells with cyanobacterial growth were determined qualitatively by presence of green coloration, and heterotroph persistence was evaluated by plating onto rich media. In the majority of cultures both cyanobacteria and the corresponding heterotroph persisted through these perturbations, although B. subtilis was lost from the co-culture somewhat more frequently than E. coli (FIG. 4E).

### Example 6: Bioproduction from Functionalized Co-Cultures

As multiple species can be co-cultured with cscB+ S. elongatus, it is possible to exchange heterotrophs to functionalize consortia for a desired activity. In this design, the heterotrophic species of the consortia acts as a “conversion module” to metabolize the products of photosynthesis into target bioproducts in a “one-pot reaction” (FIG. 5A). Two heterotrophic strains capable of producing distinct products were tested: enzymes (FIG. 5B-5C) and chemical precursors (FIG. 5D).

Alpha-amylase was produced in co-cultures of B. subtilis strain 168 and cscB+ S. elongatus (FIG. 5A-5B). B. subtilis is a chassis for enzyme production while strain 168 naturally produces active alpha-amylase. In consortia with S. elongatus, B. subtilis 168 produced alpha-amylase after 24 hours in constant light (FIG. 5B-5C). The resulting alpha-amylase was functional as determined by enzymatic assay and accumulated at significantly higher levels in co-cultures with cscB+ S. elongatus induced to secrete sucrose (FIG. 5B-5C) in comparison to other co-cultures.

Engineered E. coli/S. elongatus communities produced PHB. E. coli strains harboring a previously described PHB production plasmid, pAET41 [Peoples et al. J Biol Chem. 264:15298-303 (1989)] were co-cultured with cscB+ S. elongatus for one week in constant light and measured PHB in the total biomass by liquid chromatography (FIG. 5D). While production from the wild type. E. coli W strain is similar with and without IPTG, the ΔcscR E. coli W mutants that utilize sucrose more effectively produce significantly more P-B (FIG. 2C, FIG. 5D). Furthermore, upon the addition of IPTG, the ΔcscR E. coli W strain can produce three times as much PHB in co-culture than in un-induced consortia.

Taken together, these results demonstrate that consortia can be flexibly programmed for photoproduction of different bioproducts by employing different heterotrophic organisms.

### Example 7: Characterization of H. boliviensis Growth in Co-Culture Media

Halomonas boliviensis was selected as a potential co-culture partner with cyanobacteria partially because H. boliviensis can produce useful compounds such as hydroxylated alkyl acids. H. boliviensis also grows well under limited nutritional conditions, similar to those routinely used as cyanobacterial minimal media (see, e.g., Guzmán et al. Appl. Microbiol. Biotechnol. 84, 1069-1077 (2009); Quillaguamán et al., Appl. Microbiol. Biotechnol. 74, 981-986 (2007); Quillaguamán et al., J. Appl. Microbiol. 99, 151-157 (2005); Rivera-Terceros et al., J. Biol. Res. 22: 8 (2015).

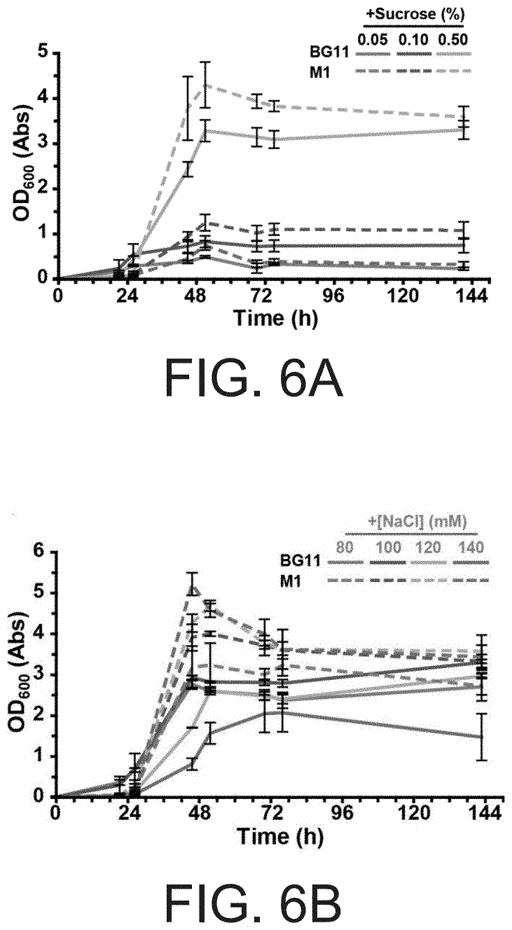

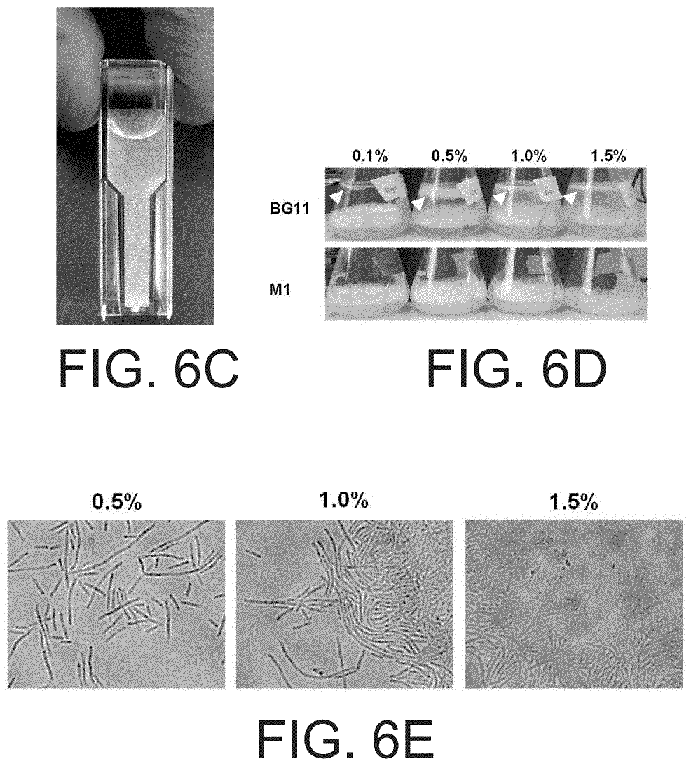

Indeed, H. boliviensis grew readily in standard BG11 medium as well as a modified BG11 medium (M1 medium; see Table 2) when supplemented with sucrose and NaCl (FIG. 6). H. boliviensis was therefore capable of growth under previously-established culturing conditions favorable to S. elongatus CscB sucrose production (Ducat et al., 2012). At a given salt concentration, H. boliviensis exhibited growth proportional to the concentration of supplemented sucrose (from 0.05% to 0.5%, w/v; FIG. 6A). However, precise quantification of H. boliviensis growth in BG11 medium was complicated by cell aggregation and adhesion to the flask, a behavior minimized in M1 medium (FIG. 6C-6E; Table 2). In M1 medium with fixed sucrose levels, increasing NaCl concentrations from 80 to 140 mM enhanced H. boliviensis growth (FIG. 6B).

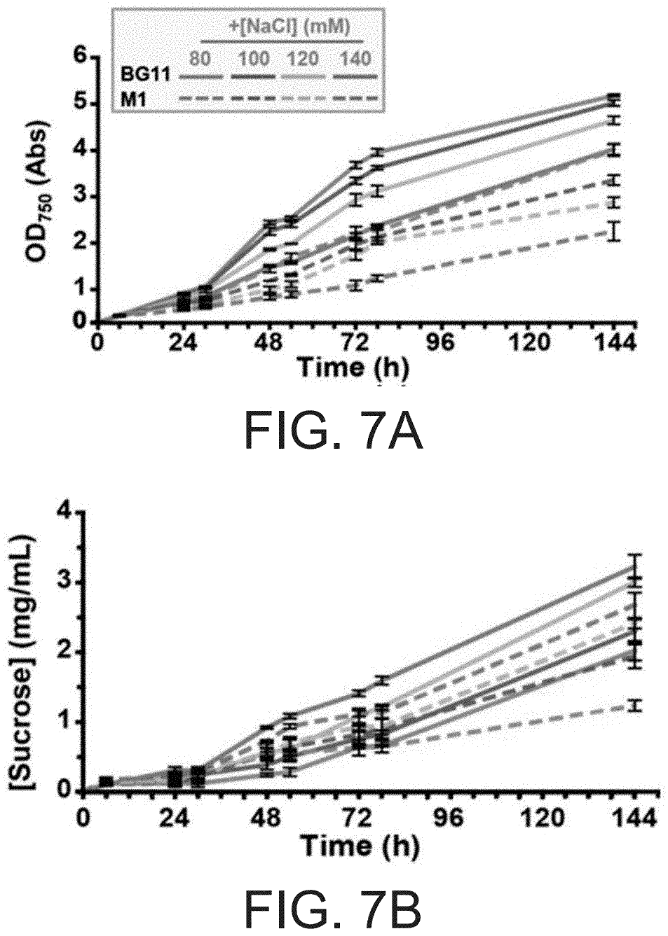

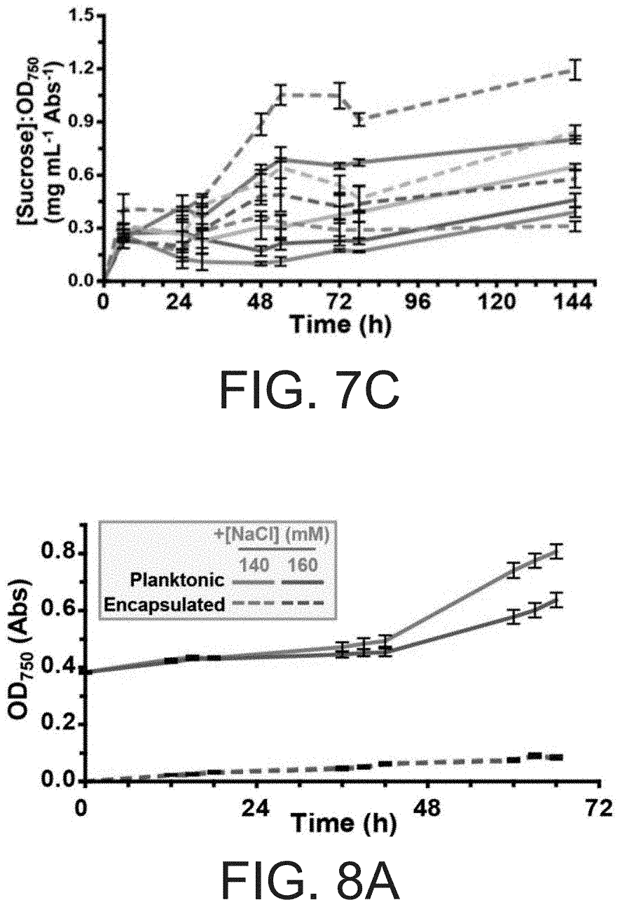

S. elongatus CscB grew in both BG11 and M1 media and produced sucrose when induced to express cscB via the addition of IPTG (FIG. 7). In agreement with the inventors' previous results (Ducat et al., 2012; Hays et al., 2017), S. elongatus CscB grew slower in BG11 with increased NaCl concentrations (FIG. 7A) while producing more sucrose (FIG. 7B). Similarly, S. elongatus CscB also grew slower in M1 media-which has about 25 mM additional Na⁺ content and increased total osmolarity (Table 2)—than in BG11 (FIG. 7A), while also producing more sucrose (FIG. 7B). Because sucrose specific productivity was superior overall in M1 (FIG. 7C), and because H. boliviensis exhibited decreased aggregation in M1 relative to BG11 (FIG. 6C-6E), M1 media (Table 1) was used for initial characterization of co-cultures.

### Example 8: Productivity of Alginate-Encapsulated S. elongatus CscB

If cyanobacteria are encapsulated, they could facilitate selective harvesting of H. boliviensis biomass from mixed co-cultures. To test this, sodium alginate-suspended S. elongatus CscB were dripped into a barium chloride gelling solution, generating spherical, barium-alginate hydrogel beads of about 2.6 mm in diameter that contained entrapped cyanobacterial cells (FIG. 8A-8D). While calcium ions are routinely used to cross-link alginate, barium ions have been shown to generate hydrogels that are both stronger and more stable over time, without negatively affecting cell viability (Ehrhart et al., PLoS One 8, 1-9 (2013); Msrch et al., Biomacromolecules 7, 1471-1480 (2006); Ehrhart et al., J. Biomed. Mater. Res.—Part A 100 A, 2939-2947 (2012).

The sucrose productivity of encapsulated S. elongatus CscB was examined relative to planktonic (i.e., “free floating”) suspensions by culturing equal numbers of free and encapsulated cyanobacteria into separate cultures. As shown in FIGS. 8A-8D, the specific productivity of sucrose from encapsulated S. elongatus CscB was sustained, and likely enhanced, relative to planktonic cells (FIGS. 8A-8D). Similar amounts of sucrose were measured in the supernatant of both encapsulated and planktonic cultures over 66 hours, with encapsulated cells exhibiting somewhat higher sucrose productivity (FIG. 8B). Similar amounts of total sucrose were produced despite the fact that planktonic cells exhibited growth and division following a brief lag (FIGS. 8A, 8C). In contrast, cultures containing Ba-alginate encapsulated S. elongatus CscB beads displayed a minimal, linear increase of OD₇₅₀ (FIG. 8A) that was mostly attributable to visible salt precipitate, but which also contained a small number of “escaped,” planktonic cyanobacteria. Because planktonic cultures were able to grow and divide, while encapsulated cells were likely restricted, the sucrose productivity of each culture was calculated on a per chlorophyll a basis, finding that encapsulated cultures exhibited an approximate 2-fold to 3-fold increase in sucrose specific productivity (FIG. 8C, 8E, 8F).

Cyanobacterial chlorophyll a concentrations are influenced by growth conditions (Muramatsu & Hihara, J. Plant Res. 125: 11-39 (2012)). Attempts were made to estimate sucrose specific productivity on a per cell basis, but unfortunately, a treatment was not identified that could both sufficiently dissolve the resilient Ba-alginate and yield viable, planktonic cells. Cyanobacterial growth was therefore examined within the alginate matrix by transmission electron microscopy (TEM) of bead cross-sections over time. Immediately following encapsulation, approximately 1 cell/pocket was observed within the alginate matrix, and this was unchanged after 7 days of cultivation (FIGS. 8H-8K). These data indicate that the alginate may act as a physical restraint that substantially arrests cell growth. After 42 days of culture, the beads typically exhibited pockets with only 6-10 cells, (FIG. 8J) further indicating that growth within the beads is extremely limited (and that the doubling time is greater than 10 days).

Assuming that the cell number did not significantly change within the beads after 66 hours of encapsulation (FIG. 8H-8I), the specific productivity of sucrose on a per OD₇₅₀ basis was estimated, indicating an approximate 2-fold increase in sucrose production per encapsulated cell, relative to planktonic cultures (FIG. 8G).

### Example 9: Stable, Long-Term Production of PHB from a Synthetic Cyanobacterium/Heterotroph Consortium

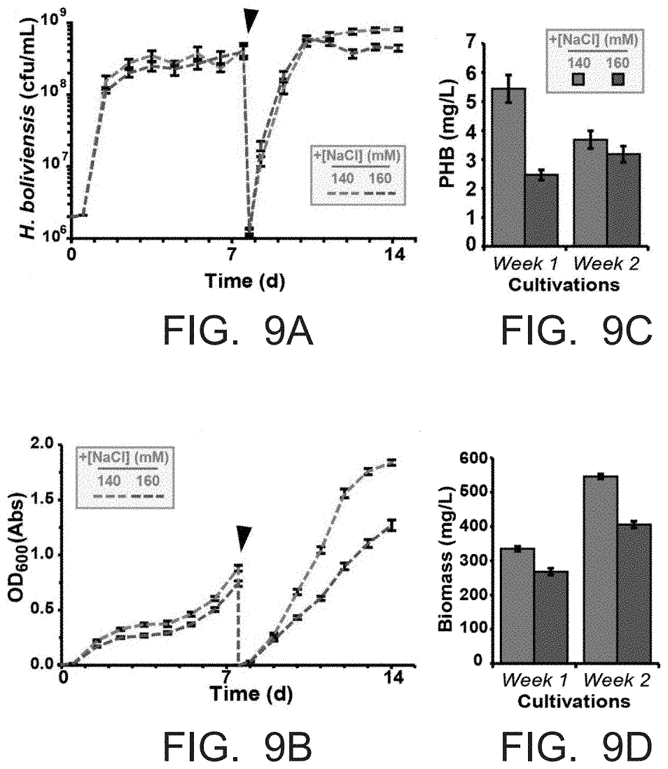

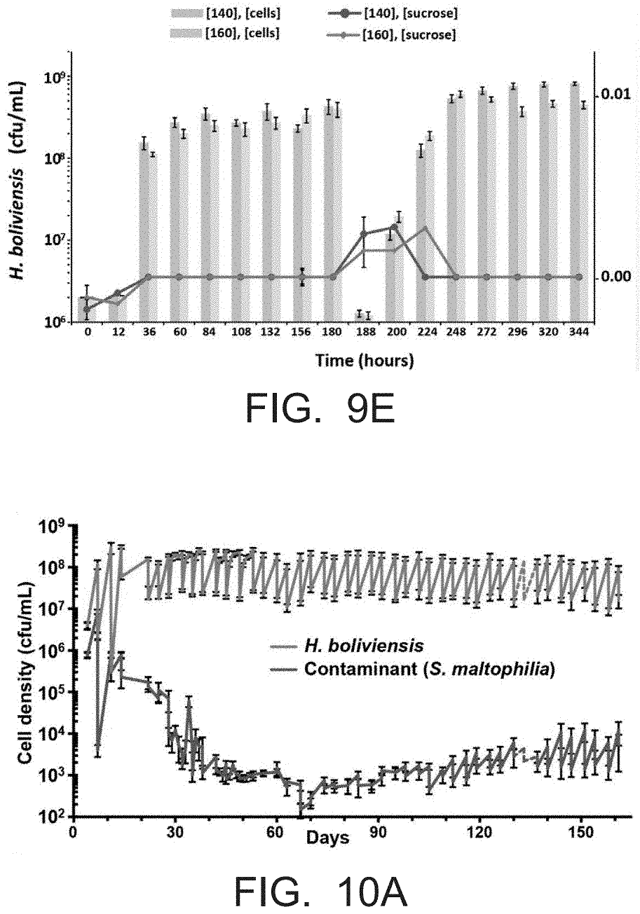

The capacity of sucrose secreted from encapsulated S. elongatus CscB was examined to support the growth of H. boliviensis in direct co-culture. Ba-alginate beads containing S. elongatus CscB were prepared as described in the Examples above and added to liquid cultures of planktonic H. boliviensis (see FIG. 1G). Both the growth of H. boliviensis and intracellular PHB content was monitored over two weeks, removing and replacing the spent liquid medium once after 7 days (FIG. 9). Analysis of H. boliviensis colony forming units (CFU) indicated that H. boliviensis was capable of growing to a high titer (0.5-1×10⁹ CFU/mL), and that cells rapidly recovered following back-dilution (FIG. 9A). Measurements of free sucrose in co-cultures indicated that H. boliviensis efficiently depleted the cyanobacterially-derived carbohydrates, usually keeping the levels below the detection limits of our assay (FIG. 9E). After about 4 days of growth, a rapid increase in OD₆₀₀ measurements (FIG. 9B) uncorrelated with increased CFU/mL—was accompanied by a “greening” of the liquid medium (data not shown), suggesting that some cyanobacteria escaped the sequestration of the alginate bead and grew planktonically.

The cellular biomass was evaluated by withdrawal of media from the liquid phase after each week, and PHB levels were determined to be between 2.5-5.5 mg PHB L⁻¹ (FIG. 9C), equating to <1% of the total harvested dry weight (FIG. 9D). The growth medium was then re-formulated for co-culture with reduced nitrogen (M2 medium; Table 2) to stimulate PHB production. The newly modified BG11 medium, M2, contained low nitrate (2 mM), additional phosphate for nutrition/buffering, and increased Mg²⁺ to reduce H. boliviensis cell adhesion (FIG. 6C-6E).

Alginate-encapsulated S. elongatus CscB in M2 medium supported robust growth of co-cultivated H. boliviensis for more than five months using only CO₂ as the input carbon source (FIG. 10). All spent liquid media containing cultivated biomass was harvested twice weekly (after 3 and 4 days) and replaced with fresh medium. These “back-dilution” events periodically increased nitrate availability, and residual H. boliviensis cells were observed that exhibited rapid growth in the refreshed media, as measured by CFU counts (FIG. 10A) or OD₆₀₀ (FIG. 10B). Furthermore, H. boliviensis growth rates were consistent following each back-dilution (doubling-time of ˜10 h; FIG. 10C). Such consistency also indicated that encapsulated cyanobacteria retained viability throughout the co-culture experiment, since cyanobacterial photoassimilate represents the only available organic carbon source. Importantly, the alginate beads remained intact and green for the entire 5.5 months of the co-culture (FIG. 8G) and did not display characteristics of chlorophyll and degradation, which is typically observed within 24 hours of nitrogen depletion (Barker-Åström et al., Arch. Microbiol. 183: 66-69 (2005); Hickman et al., Algal Res. 2: 98-106 (2013; Lahmi et al., J. Bacteriol. 188: 5258-5265 (2006). Indeed, although free nitrogen was rapidly depleted to undetectable levels within the 24 hours following each back-dilution event (see Example 1), visible pigmentation changes were only observed in encapsulated S. elongatus during an extended (8 day) period of nitrogen depletion. Initially, the encapsulated cyanobacteria have a dark green hue (at Day 0 of nitrogen depletion) that slowly decreases and then slowly returns to nearly the same intensity before stabilizing. Though cell densities within the beads could not be ascertained, this color change apparently reflects a change in the overall chlorophyll a concentration within the beads over time. Color change from Day 12 to Day 20 is especially pronounced because of an extended nitrogen deprivation period; Day 23 and Day 26 reflect recovery despite ongoing back-dilution and periods of nitrogen depletion.

These data indicate that alginate encapsulation delayed the onset of nitrogen stress responses relative to planktonic cells, perhaps because alginate encapsulation restricts growth and thereby reduces cellular nitrogen requirements (as seen in other encapsulated cells, see e.g., Kaya & Picard, Biotechnol. Bioeng. 46, 459-464 (1995). Indeed, intact S. elongatus CscB cells were observed by TEM at greater than 150 days-post encapsulation, although nitrogen was limited for the majority of the cultivation period (see FIG. 8K).

Co-cultivated H. boliviensis produced high titers of PHB and productivity during five months of co-culture, as monitored by brightfield microcopy, Nile red staining, electron microcopy, and chromatography. Following each back-dilution, large, light-diffracting bodies accumulated in H. boliviensis, and these inclusions stained with the hydrophobic fluorescent dye, Nile red (FIG. 10E). These bodies correlated with large cellular inclusions reminiscent of PHB granules visualized via TEM (FIG. 10E; (Quillaguamin et al., Enzyme Microb. Technol. 38, 148-154 (2006)). Finally, HPLC analysis of harvested biomass revealed consistent PHB production from Day 25 to Day 170 (FIG. 10F)—the time during which the contribution of contamination was negligible (see paragraph below)—with an average of 19.5±0.7 mg PHB L⁻¹ d⁻¹ (SEM; n=40), from 63.3±1.8 mg dw L⁻¹ d⁻¹ (SEM; n=40) of cell biomass (FIG. 10G). This equates to an average of 30.6±0.5% PHB/dry weight biomass (SEM; n=40; FIG. 10G), a ˜30-fold increase in production relative to M1 media (FIG. 9A-9D). Maximal PHB productivity, 28.3 mg PHB L⁻¹ d⁻¹, from 36.8% PHB-content biomass, was observed after 90 days (FIG. 10F).

In addition to productivity, S. elongatus CscB/H. boliviensis co-cultures also demonstrated resistance to both perturbation and contamination. Encapsulated cyanobacterial cells remained viable through repeated cycles of nitrogen availability and also were stable through a deliberate extended period of nitrogen deprivation (FIGS. 10A-10B). Indeed, H. boliviensis total cell biomass and PHB increased during the months-long co-culture of periodic nitrogen availability (FIG. 10F). One likely interpretation is that S. elongatus CscB production of photoassimilate increased over time due to slow division within the alginate beads: a hypothesis supported by TEM imaging of Ba-alginate beads at more than 7 days after cyanobacterial encapsulation (FIGS. 8H-8K). All co-cultures were maintained in the absence of antibiotic selection and were therefore susceptible to contamination. Suspensions of each co-culture were routinely plated on both H. boliviensis-selective hypersaline 1097 medium plates and non-selective TSB plates to identify potential contaminating microbes (FIG. 10A). Detection of exogenous microbes was sometimes observed; for example, in the long-term experiments presented in FIG. 10A, CFU counts indicated significant initial contamination, which was attributed to the sucrose-utilizing microbe, Stenotrophomonas maltophilia, by 16s rRNA sequencing. Yet in the absence of other selective pressures, simple back-dilution was sufficient to support H. boliviensis at cell densities between 10⁷ to 10⁸ cells/mL, while S. maltophilia, declined four-orders of magnitude to steady state cell densities <10³ cells/mL (barely above the detection limits of our plating assay). Twice-weekly microscopy observation and Nile red staining also indicated that co-culture contamination was minimal (FIG. 10D). As the contamination was minimal and harvested cell pellets lacked the visible greening of cyanobacterial cells, it is likely that the vast majority of cell biomass harvested was H. boliviensis, which robustly sustained PHB production over months of co-culture (FIG. 10D-10G).

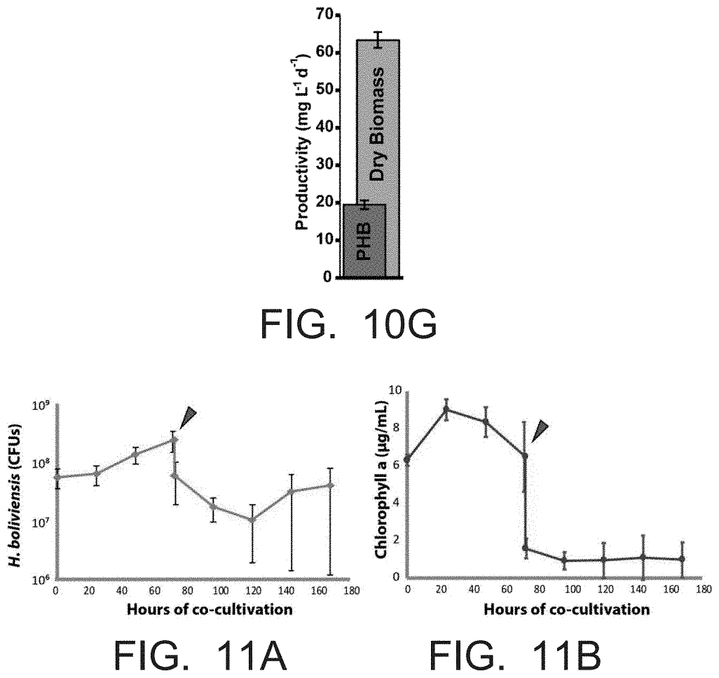

To further evaluate the promising stability and productivity observed in synthetic co-cultures (FIG. 10), and the relative utility of alginate encapsulation and H. boliviensis, the performance of reference co-cultures was examined. First, purely planktonic co-cultures of S. elongatus CscB and H. boliviensis were analyzed, keeping experimental conditions identical to those of the dataset shown in FIG. 10, except that S. elongatus CscB cells were not encapsulated within barium alginate beads. Although, H. boliviensis initially grew in such planktonic co-cultures, recovery was poor following a 1:4 back-dilution of M2 media at 72 hours post-inoculation (FIG. 11A). This was likely due to reduced viability of their partner cyanobacteria, as S. elongatus CscB cells were visibly observed to undergo “bleaching” that is characteristic of prolonged exposure to low nitrogen. The cyanobacterial chlorosis was quantified by extracting chlorophyll a every 24 h of the co-culture (FIG. 11B), observing that chlorophyll density decreased sharply after 24 h and did not recover thereafter. In total, results indicate instability in planktonic co-cultures relative to the comparison experiments conducted with alginate encapsulation. Neither H. boliviensis, nor cyanobacterial cell pigmentation recovered following a single dilution event. Alginate encapsulation can therefore reduce the stress experienced by S. elongatus cells due to nitrogen deprivation, perhaps by restricting growth (FIG. 8H-8K) and thereby reducing nitrogen requirements.

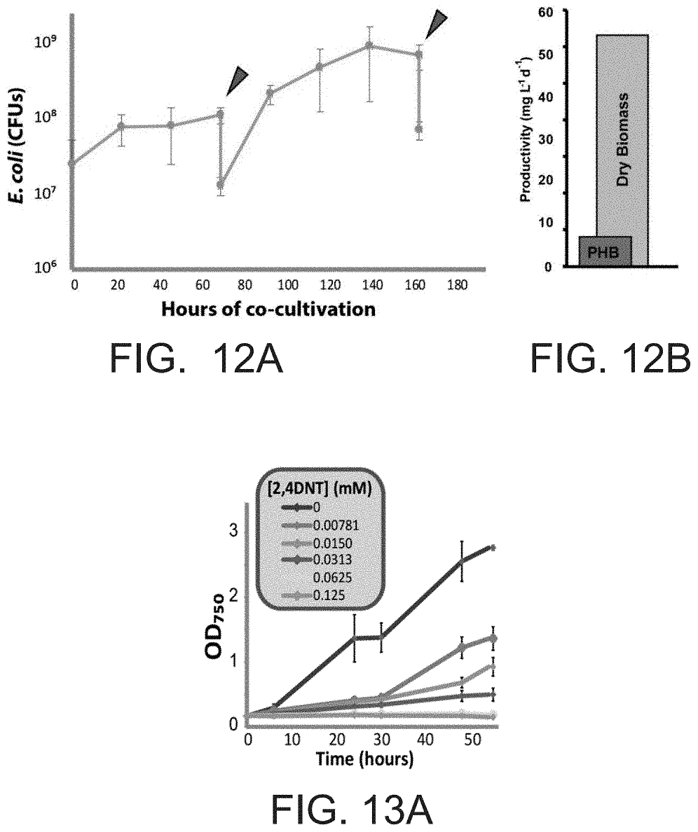

To compare the effectiveness of H. boliviensis for PHB production relative to the co-culture work illustrated in Examples 2-7, the co-culture conditions represented in FIG. 10 were replicated, but a strain of E. coli W deleted for the sucrose catabolism operon repressor (ΔcscR) was and heterologously expressing the phbABC operon (Peoples et al., 1989) substituted to enable PHB production. The ΔcscR E. coli W and phbABC cells were able to grow readily when co-cultured with encapsulated S. elongatus CscB (FIG. 12A), achieving biomass dry weights over the course of a 7-day experiment that were comparable to H. boliviensis and that contained at least about 10% PHB (FIG. 12B).

### Example 10: Co-cultures Facilitate Environmental Clean-Up of Pollutants

This Example illustrates that encapsulated S. elongatus CscB+ cells are more tolerant of the toxin 2,4-dinitrotoluene (2,4-DNT) than non-encapsulated S. elongatus CscB⁺ cells, and that co-cultures of these cyanobacteria with dnt-transgenic Pseudomonas putida can degrade dinitrotoluenes (e.g., 2,4-DNT). Such dinitrotoluenes can be environmental pollutants from industrial activities such as from the manufacture of the explosive trinitrotoluene (TNT).

The transgenic Pseudononas putida have the dnt the operon (expression cassette; pSEVA221-cscRABY) that was integrated in order to confer the capacity to degrade 2,4-DNT. This operon is derived from Burkholderia cepacia sp. R34 This dnt expression cassette includes nucleic acid segments encoding the DntA, DntB, DntD, DntE, DntG and DntR enzymes. Nucleic acid segments in the pSEVA221-cscRABY have the following sequences.

The nucleic acid segment encoding DntR (LysR-type regulatory protein) has the following sequence (SEQ ID NO:16).

| | |
|---|---|
| 1 | ATGGATCTGC GCGACATCGA CTTGAATCTG CTGGTGGTCT |
| 41 | TCAACCAGCT ACTGCTCGAC CGGAGCGTAT CGACGGCCGG |
| 81 | CGAAAAACTG GGGCTGACGC AGCCTGCCGT CAGTAATTCA |
| 121 | CTTAAACGGC TGCGTACAGC GCTGAACGAC GATTTGTTCT |
| 161 | TGCGCACCTC AAAAGGCATG GAGCCGACAC CGTATGCACT |
| 201 | GCATCTTGCG GAGCCCGTGA TCTATGCGCT CAACACGCTG |
| 241 | CAGACGGCAC TGACGACCCG TGACTCTTTC GACCCATTTG |
| 281 | CCAGCACGCG CACCTTCAAC TTGGCAATGA CCGACATCGG |
| 321 | CGAGATGTAC TTCATGCCCC CACTGATGGA AGCGCTTGCG |
| 361 | CAACGAGCTC CTCACATCCA GATCAGCACG CTGCGCCCGA |
| 401 | ATGCTGGCAA TCTGAAGGAG GATATGGAGT CCGGTGCGGT |
| 441 | TGATCTCGCC TTGGGTCTTC TGCCAGAGCT ACAGACCGGA |
| 481 | TTCTTCCAGC GGCGCCTCTT TCGCCACCGC TACGTATGCA |
| 521 | TGTTCCGCAA GGACCATCCA AGCGCCAAAT CCCCCATGAG |
| 561 | CCTGAAACAG TTCAGTGAAC TGGAGCATGT CGGCGTGGTC |
| 601 | GCACTCAACA CCGGACACGG TGAGGTCGAT GGCCTGCTCG |
| 641 | AACGCGCAGG CATCAAAAGG CGCATGCGGC TGGTGGTGCC |
| 681 | GCATTTCATT GCGATCGGCC CCATTCTGCA CAGCACCGAC |
| 721 | CTCATCGCGA CCGTGCCGCA GCGTTTTGCC GTTCGCTGCG |
| 761 | AAGTGCCTTT TGGTCTGACG ACATCCCCGC ACCCGGCCAA |
| 801 | GCTGCCTGAC ATCGCCATCA ACCTGTTTTG GCATGCCAAG |
| 841 | TACAACCGGG ATCCGGGCAA CATGTGGCTA CGTCAGTTGT |
| 881 | TCGTCGAGCT TTTCTCTGAA GCATAA |

The nucleic acid segment encoding DntAa (2,4-dinitrotoluene dioxygenase system ferredoxin—NAD(+), reductase component) has the following sequence (SEQ ID NO: 17).

| | |
|---|---|
| 1 | ATGGAACTGG TAGTAGAACC CCTCAATTTG CATCTGAACG |
| 41 | CGGAGACCGG CAGCACCCTG CTTGACGTGC TCAGGTCCAA |
| 81 | CGAGGTCCCC ATTTCTTATA GCTGCATGTC GGGCCGCTGC |
| 121 | GGCACTTGCC GTTGCCGTGT GATTGCCGGC CATCTTCGCG |
| 161 | ATAACGGCCC CGAGACAGGG CGCCCGCAGG CAGGAAAGGG |
| 201 | GGCCTATGTC CTGGCCTGTC AGGCGGTTCT GACCGAAGAC |
| 241 | TGCACGATCG AGATTCCTGA ATCTGACGAG ATCGTGGTTC |
| 281 | ACCCGGCGCG CATCGTCAAG GGGACGGTCA CAGCGATAGA |
| 321 | CGAAGCCACC CATGACATCC GGCGCCTGCG CATCAAACTG |
| 361 | GCCAAACCGC TTGAGTTCAG CCCTGGCCAG TACGCAACGG |
| 401 | TGCAGTTCAC GCCCGAATGC GTCCGCCCCT ATTCGATGGC |
| 441 | CGGGCTGCCT AGCGATGCGG AAATGGAGTT TCAGATTCGC |
| 481 | GCGGTTCCGG GCGGGCATGT CAGCAACTAC GTTTTCAATG |
| 521 | AACTGTCCGT AGGCGCTTCG GTGCGGATCA GCGGCCCCCT |
| 561 | CGGAACGGCC TATCTGCGGC GCACGCACAC CGGCCCCATG |
| 601 | CTTTGTGTGG GGGGTGGAAC AGGTCTGGCG CCCGTCCTTT |
| 641 | CGATCGTTCG AGGCGCACTG GAAAGCGGGA TGAGCAACCC |
| 681 | CATCCATCTG TACTTCGGTG TGCGGAGCGA GCAGGACATC |
| 721 | TATGACGAGG AACGCCTTCA CGCATTGGCT GCAAGGTTTC |
| 761 | CGAATCTCAA GGTGAATGTC GTTGTTGCAA CAGGCCCTGC |
| 801 | CGGCCCTGGT CATCGATCCG GCCTGGTCAC CGATCTGATC |
| 841 | GGCCGTGACT TGCCCAATTT GGCGGGATGG CGCGCCTACC |
| 881 | TGTGTGGCGC TCCGGCCATG GTCGAGGCCC TGAACCTGCT |
| 921 | CGTTGCTCGC CTAGGCATAG TACCCGGGCA CATCCATGCC |
| 961 | GATGCGTTCT ATCCCAGCGG CGTCTGA |

The nucleic acid segment encoding DntAb (2,4-dinitrotoluene dioxygenase system, ferredoxin component) has the following sequence (SEQ ID NO:18).

| | |
|---|---|
| 1 | ATGAGCGAGA ACTGGATCGA CGCCGCCGCC CGCGACGAGG |
| 41 | TGCCCGAGGG CGACGTGATC GGCATCAATA TCGTCGGCAA |
| 81 | GGAGATTGCC CTCTACGAGG TGGCGGGCGA GATCTACGCC |
| 121 | ACCGACAACA CCTGCACTCA CGGCGCCGCC CGCATGAGCG |
| 161 | ATGGCTTTCT CGAAGGCCGG GAAATTGAAT GTCCTTTGCA |
| 201 | TCAAGGCCGA TTCGATGTTT GCACGGGTAA AGCCTTGTGC |
| 241 | ACACCCCTGA CACAGGACAT CAAAACCTAC CCCGTAAAAA |
| 281 | TCGAAAACAT GCGCGTGATG CTCAAGCTGG ACTAA |

The nucleic acid segment encoding DntAc (2,4-dinitrotoluene dioxygenase system, large oxygenase component) has the following sequence (SEQ ID NO:19).

| | |
|---|---|
| 1 | ATGAGTTACC AAAACTTAGT GAGTGAAGCA GGGCTGACGC |
| 41 | AAAAGCACCT GATTTATGGC GACAAAGAAC TTTTCCAGCA |
| 81 | CGAATTGAAG ACCATCTTCG CGCGGAACTG GCTTTTTCTG |
| 121 | ACCCATGACA GTCTGATTCC CTCCCCCGGC GACTATGTCA |
| 161 | AAGCCAAAAT GGGCGTCGAT GAAGTCATCG TCTCCCGCCA |
| 201 | GAACGATGGC TCGGTGCGAG CCTTTTTGAA TGTTTGCCGT |
| 241 | CACCGGGGCA AGACAATAGT TGACGCTGAA GCCGGAAATG |
| 281 | CGAAAGGCTT TGTGTGCGGT TACCACGGCT GGGGCTATGG |
| 321 | CTCCAACGGC GAACTGCAAA GCGTTCCCTT TGAAAAAGAG |
| 361 | TTGTACGGAG ATGCGATCAA AAAGAAATGC CTGGGCTTGA |
| 401 | AAGAAGTCCC CCGCATCGAA AGCTTTCATG GCTTTATCTA |
| 441 | TGGCTGTTTT GATGCAGAAG CTCCCCCGCT CATCGATTAT |
| 481 | CTGGGTGATG CAGCCTGGTA CCTGGAACCC ACCTTCAAGC |
| 521 | ACTCTGGTGG CCTGGAACTT GTAGGCCCCC CCGGCAAAGT |
| 561 | GGTGGTTAAG GCCAACTGGA AGCCTCTTGC GGAAAACTTT |
| 601 | GTAGGTGACG TCTACCACAT TGGTTGGACG CACGCATCTA |
| 641 | TTTTGCGCGC AGGGCAGTCG ATATTTGCTC CTCTTGCGGG |
| 681 | CAACGCTATG TTTCCACCCG AAGGCGCGGG CTTGCAAATG |
| 721 | ACCACCAAGT ATGGCAGTGG AATTGGCGTA TTGTGGGACG |
| 761 | CCTACTCCGG TATCCAGAGC GCTGATATGG TTCCCGAAAT |
| 801 | GATGGCATTC GGCGGCGCAA AACAGGAAAA GCTCGCCAAA |
| 841 | GAAATCGGCG ATGTCCGGGC GCGGATTTAC CGCAGCCAAC |
| 881 | TGAACGGCAC GGTTTTCCCG AACAACAGCT TTTTGACCTG |
| 921 | CTCCGGTGTC TTCAAGGTCT TTAACCCGAT CGATGAAAAC |
| 961 | ACGACCGAGG TTTGGACGTA TGCCATCGTA GAAAAAGACA |
| 1001 | TGCCTGAGGA CTTAAAGCGT CGCTTGGCTG ACGCGGTTCA |
| 1041 | GCGCAGTGTC GGACCAGCAG GATACTGGGA AAGCGACGAC |
| 1081 | AACGACAACA TGGGGACGTT GTCGCAAAAT GCCAAGAAAT |
| 1121 | ACCAATCCAG CAACAGTGAT CTGATTGCCG ATTTGGGTTT |
| 1161 | CGGCAAGGAC GTCTACGGCG ACGAATGCTA TCCGGGCGTC |
| 1201 | GTTGGCAAAT CGGCAATCAG CGAAACCAGC TATCGCGGAT |
| 1241 | TCTACCGTGC CTACCAGGCT CACATCAGCA GCTCCAATTG |
| 1281 | GGCCGAGTTC GAAAACACCT CCCGAAATTG GCACACCGAA |
| 1321 | CTCACCAAGA CGACTGATCG CTAA |

The nucleic acid segment encoding DntAd (2,4-dinitrotoluene dioxygenase system, small oxygenase component) has the following sequence (SEQ ID NO:20).

| | |
|---|---|
| 1 | ATGATGATCA ATACCCAGGA AGACAAGCTG GTCTCCGCGC |
| 41 | ACGACGCCGA AGAATTTCAC CGTTTCTTCG TCGGGCACGA |
| 81 | CAGCGATCTG CAGCAAGAAG TCACCACACT CCTGACCCGC |
| 121 | GAAGCGCACC TGCTGGACAT TCAGGCCTAC AAAGCCTGGC |
| 161 | TTGAACACTG CGTTGCCCCC GAGATCAAAT ACCAAGTGAT |
| 201 | CTCGCGAGAA CTTCGCTCCA CTTCCGAGCG TCGATACCAA |
| 241 | CTGAATGATG CGGTGAATAT CTACAACGAG AACTATCAAC |
| 281 | AGCTGAAAGT TCGAGTTGAA CACCAGATGG ATCCTCAGAA |
| 321 | CTGGTACAAC AGCCCGAAGA TCCGCTTCAC CCGCTTCGTC |
| 361 | ACCAATGTCA CGGCGGCCAA GGACAAGAGC GCACCGGAAA |
| 401 | TGCTGCATGT GCGGTCCAAC CTCATTCTCC ATCGCGCCAG |
| 441 | ACGAGGAAAC CAAGTTGACG TCTTCTATGC AACGCGAGAA |
| 481 | GACAAATGGA AACGCATCGA AGGTGGTGGC ATCAAATTGG |
| 521 | TCGAACGCTT TGTGGACTAC CCGGAGCGCA GTCCCCAAAC |
| 561 | CCACAACCTG ATAATCTTCC TGTGA |

The nucleic acid segment encoding DntB (4-methyl-5-nitrocatechol 5-monooxygenase) has the following sequence (SEQ ID NO:21).

| | |
|---|---|
| 1 | GTGCATCACG TTTCTACTAA GTCGCCGTCT ACCTTGTCGG |
| 41 | CGGAGTGTGA AGTTCTCATC GTCGGGGGTA GCTTGGTCGG |
| 81 | CTTGTCGCTT GCAAACTTTC TCGGCCACCA CGGCGTAAGC |
| 121 | GCTGCAGTCG TCGAGCGGCA CAAGGGAACG GCCATCCACC |
| 161 | CTCGTGCTGG CCACTTTCAC CTGAGGACCA TTGAAGCATT |
| 201 | TCGATATGCA GGAATCGAGC CAGAAGTCAT GCAGGAGTCT |
| 241 | CTTCGACAGT TCGATCCGGA CGGCGGTATC AACGTCGTCG |
| 281 | AATCGCTTGC CGGCAAGGAG ATCGCCAGCC TGATTGGCAA |
| 321 | CTTGAACGAA GGCGTCGAAA AACTCAGTCC AAGTAAGCGC |
| 361 | CTGTTCATGA CACAACAAAG TCTCGAACCC TTGCTTAGAA |
| 401 | AGAACGCTGA AAAGCTGGGG GCCCAACTTA ACTACCAGAT |
| 441 | GGAATTGGTT TCATTCGAGC AGGACGCCAC GGGTGTGACT |
| 481 | GCGCGAGTCA GGTACATCCC ATCCGGGGCC GTGAGCCAAG |
| 521 | TGCGTGCCAA GTACCTAATC GCCGCCGACG GTAACCGCAG |
| 561 | CCCAGTCCGA GAGAAGCTCG GAATTGAAAT GCGCGGCTAC |
| 601 | GGATTACTCT CCAACAGTAT CACCATCTAC TTCAAGGCGG |
| 641 | ACTGCACAAA ATGGATGGCT GGACGCAACC TCGGCGTGGT |
| 681 | CTATGTCAAC AATCCTGACG TTCGCGGCTT CTTCCGCTTG |
| 721 | ACGCGGGAGG CCAAGAGTGG CTTCTTGGGT GTGAACACCG |
| 761 | TAGGAGATGT CAGCCGCCCG GAGGCGAACA ATGTCGCTGA |
| 801 | AGGTATCACG GCAGAGCGCT GCGTCGAAAT TGTACGTTCC |
| 841 | GCTGTGGGCA TTCCCGATCT GGAAGTTGAA ATTGAGGGCA |
| 881 | TTGCCCCGTG GCGCGCCGTT GCCGATGTGG CTGACCGTTA |
| 921 | CAGAAGTGGA AATGTGTTTC TCATCGGCGA CGCCGCACAC |
| 961 | GTCGTTCCTC CTACCGGCGG ATTCGGCGGG AACACTGGTG |
| 1041 | TTCAGGACGC GCACAACCTT GGTTGGAAGC TGGCTTCAGT |
| 1081 | ACTAAAGGGT CAAGCAGGGC CGGCTCTGCT CGACACCTAC |
| 1121 | GAGGAGGAGC GGCGCCCGGT CGGTCAACTT ACGATCGAGC |
| 1161 | AGGCCTATTC CCGATATGTG CTGCGGATCG CACCTGAACT |
| 1201 | GGGCCGTGAA ACGATGAAGC CTGTGGTCGA CGACTTGAGC |
| 1241 | ATGGAAATTG GCTACCGCTA TTTCTCATCA GCCATCTTGT |
| 1281 | CGAACGAAAA GCGGGGAGAT CGTGTTTACG TTGATCCGCG |
| 1321 | CACGTCGTTC AGCTTGCCTG GGACCAGAGT GGGACACCTC |
| 1361 | GTCTTTCAGC GAGACGGGAA ATCGATGGCG ACCCTGGACG |
| 1401 | TTTGTGCCGG TGGTATGACC CTTCTTGCCG GAGCGGGCGG |
| 1441 | CGTCGCCTGG TGTCAGTCGA CGACTGAAGC TGCCGTAAAG |
| 1481 | CTGGGTATCG AGGTCGAGTC GAACGTCATT GGAAACGCGG |
| 1521 | GTGGACTGAC AGATGTTTCA GGTCGCGCGC TCGAGGTTCT |
| 1561 | CGGAATCGAA AGCGCAGGTG CAATTCTGGT GCGCCCCGAC |
| 1601 | GGTTTCGTGG CTTGGCGCTC AGAACCCGGT GAGGCCGCGA |
| 1641 | GTGTCGCGAG GATGATCAAC GTGCTGACCG CTGTAATGTG |
| 1681 | TCTCCAGAGT CAGCGCGTAG ATGCATCGGT CGTAGCTGCC |
| 1721 | TAA |

The nucleic acid segment encoding DntD (trihydroxytoluene oxygenase) has the following sequence (SEQ ID NO:22).

| | |
|---|---|
| 1 | ATGGCCATCA CCGACATTGC CTATTTGGTA AACGACCACA |
| 41 | CCGACCTGGA AGCGGCAGAG CGCTTCTACA CGGACTTCGG |
| 81 | CTTGAAGGTC GCCTACCGCA CCGCTGACGA GATCGGCTTT |
| 121 | CGACCCGCTC TGGCGCGTGG TTACTGCTAC GTGGCACGCA |
| 161 | AATCGAGCAA GCCCGGTCTG CGCGCAATTG CCTTCACCGC |
| 201 | CAGCACGCGG GCCGATCTGG ATGTCGCGGC GCGTTTCCCT |
| 241 | GAGGCATCGC CCATCACGCC AATCGAGCGT GAAGGTGGTG |
| 281 | GCGATAAAGT GATGCTCCAG AGCCCGGACG GCCTGCCGTT |
| 321 | CGAGATCGTG CACGGCATCC ATTCCTACGA GGAACTGGCG |
| 361 | GTGCGCCCCG CCTTGACGTT CAATACGGGC CGCGTCAAAC |
| 401 | GCCGCCACGG AGAGTTCCAA CGCCCGCCGC TGGAGCCAGC |
| 441 | GCCCATCTTG CGGCTGGGGC ACGTCGCCCT GCTGACGAAC |
| 481 | GACTTCAAGC GCAATTTCGA GTGGATGCAG TCGCGTCTCG |
| 521 | GACTACGGCC GACCGACACC ATGTATGGCA AGGACAAGAA |
| 561 | TGACCTCGTC GGCTCATTCA TGCACCTTTC CGGCGGTGGC |
| 601 | GAGTGGACAG ATCATCACTC GCTTGCCCTT TTCCCGGACC |
| 641 | CGAGTCCCCG CGTCCACCAC TTCTCGTTCG AGGTGGAGGA |
| 681 | CCTCGATGCG CAGGGAATGG GAAACCAATG GCTGATCAAG |
| 721 | CACGGGTGGA AGCATACCTG GGGCATTGGT CGTCACATAT |
| 761 | ACGGCAGCCA GATCTTCGAT TACTGGTTCG ATCCCGACGG |
| 801 | CAACATTGTC GAGCATTTCA CCGATGGCGA CCTTGTCCGT |
| 841 | CCCGGCTGTA AGCCAGGCTT GGTCGCGCTC GACGAGTCGC |
| 881 | TCTTTGCTTG GGGTCCGCCG ATGCAGCCCG CCAACTTCGT |
| 921 | CGAACTGGCT GCACACCACG AATGA |

The nucleic acid segment encoding DntE (methylmalonate-semialdehyde dehydrogenase) has the following sequence (SEQ ID NO:23).

| | |
|---|---|
| 1 | ATGTCTGCAG TTCGAGAGTC GCTCAGTGAT CAAGTCCCCA |
| 41 | CCGTCAAACT ACTGATCGAC GGCAAGTTCG TCGATTCCAA |
| 81 | TACGACGGAA TGGCTGGATG ACGTTAATCC GGCGACGCAG |
| 121 | GCCGTTCTGT CACGTGTCCC TATGGCAACC GCCGAAGAGG |
| 161 | TGAACGCCGC CGTTGCGGCA GCCAAGCGCG CATTCGCAAG |
| 201 | CTGGAGGCAC GCACCAATCG GGACAAGGGC GCGAATTTTC |
| 241 | CTCAAGTACC AGCAACTGAT TCGGGAAAAC ATGGGGGAAC |
| 281 | TGGCCGCATT GCTGACGGCT GAGCAAGGCA AGACCCGATT |
| 321 | GGATGCCGAA GGCGACATCT TCCGCGGACT CGAGGTGGTT |
| 361 | GAACACGCTG CGGCCATCGG GAACCTGCAA CTAGGCGAAC |
| 401 | TTGCCAACAA CGTTGCCAAG ATGGTGGATA CGTACACGCG |
| 441 | TCTGCAGCCC ATCGGTGTTT GTGCGGGCAT CACGCCATTC |
| 481 | AACTTCCCTG CAATGATTCC GCTGTGGATG TTTCCGATGG |
| 521 | CTATCGCATG CGGCAACACC TTCGTGCTCA AGCCATCCGA |
| 561 | GCAGGATCCC ATGGTGACCA TGCGCCTCGT CGAACTGGCG |
| 601 | CTCGAGGCCG GCGTCCCATC CGGTGTTCTG AACGTGGTAC |
| 641 | ATGGCGGAGC GCAGGTCGTT GACGCTATCT GCGATCATCC |
| 681 | CGACATCAAG GCGATCTCGT TTGTGGGGTC CACCCGTGTA |
| 721 | GGCACGCATG TCTACAACCG GGCCACGCTG GCAGGCAAGC |
| 761 | GTGTGCAATG CATGATGGGC GCCAAGAATC ACGCGATCCT |
| 801 | CATGCCCGAT GCCAACCGGG AGCAGTCGCT GAATGCGATC |
| 841 | GCGGGCGCCG CTTTCGGCGC TGCTGGCCAG CGTTGCATGG |
| 881 | CTCTGCCGGT GCTGGTCATG GTTGGCGAGG CACAAAGGTG |
| 921 | GCTGCCCGAT TTGGTCGCCA AGGCCAAGGC ACTGACCGTC |
| 961 | AATGCCGGCG ATGCAGCAGG TGCCGATGTT GGTCCGCTTA |
| 1001 | TCAGCCCATC CGCGTGTGGC CGAGTTCGTG AACTAATTGG |
| 1041 | TGAGGGCGTT GAGGCTGGCG CCAAGCTCGA ACTGGATGGC |
| 1081 | CGTGCTGTCA GCGTCTCCGG GTACGAGAAA GGCAACTTCG |
| 1121 | TCGGCCCGAC CATCTTTTCT GGCGTCAAGC CGGGAATGTC |
| 1161 | AATCTATGAC ATCGAGATCT TCGGCCCCGT GCTCTGCGTG |
| 1201 | ATTGCGGCGG ACAACCTCGA CGAGGCGATC GAGATCATCA |
| 1241 | ATGCCAATCC GAGCGGGAAC GGCACCGCCA TTTTCACGCA |
| 1281 | GTCGGGTGCA GCGGCGCGCA GGTTCGAAGA GGATATCGAC |
| 1321 | GTTGGACAGG TAGGCATCAA TGTGCCGATC CCCGTGCCTG |
| 1401 | TTCCGATGTT CTCCTTTACT GGCTCTCGCG CCTCGAAGCT |
| 1441 | GGGCGATCTC GGTCCTTACG GCAAGCAGGT CATCATGTTC |
| 1481 | TACACGCAGA CCAAGACCAT CACCGCACGC TGGTTCGACG |
| 1521 | ATGCCAGCGT CCGCAAGAAC GTGAACACGA CCATCGCGTT |
| 1561 | GGACTGA |

The nucleic acid segment encoding DntG has the following sequence (SEQ ID NO:24).

| | |
|---|---|
| 1 | ATGAAATTGG CCAGCTTTTT GTTGGATGGG AAGCGTCGCA |
| 41 | GTGGGGCCTT GACTGACAAG GGCCTCGCAG TGTTCGATGT |
| 81 | CGACGGCGAC ATCGGTGACT TGCTGCGCAA AGGGACCGAC |
| 121 | CTCGCGGGCT TCGACGCATT GCTCGCCAAG AGCTCGCGCA |
| 161 | TCGTCGACGC GGCCAGTGTG ACCTTTGTGC AACCGTCGGC |
| 201 | CGAGCCGCCC AAAATCTTCT GTGTGGGATT GAACTACGCT |
| 241 | GATCACACGG CCGAGAGTCC GTACGAACAG CCCGACTATC |
| 281 | CGACCATCTT TTTGCGCGTG GCTACCGCTA CGACGGGGCA |
| 321 | CGGCGCGAGT ATTGCCTTGC CTGATCTGAG CGAGCAACTC |
| 361 | GACTATGAAG GCGAGATGAT TGCTGTGCTG GGCAAGGGTG |
| 401 | GCAAGCGTAT CCCGAAGGAG CGTGCGCATG AGCACGTGTT |
| 441 | TGGCTACGCT GTCGGCAACG AGGTGTCGGT TCGCGACTAC |
| 481 | CAGTTCAAGT CGCCGCAGTG GACCATCGGC AAGAACTTCG |
| 521 | ACGGCACCGG AGCATGGGGA CCGTACATCG TCACCGCCGA |
| 561 | CGAACTCCCG CTGGGGGGAC ATGGTCTGAA GATCGAGGTT |
| 601 | CGCCTGAACG GCAAGACGGT TCAGTCATCG AATACCGGGC |
| 641 | ACATGATCTT TGACGTCGCC ACCATCATCT CGACCATCAG |
| 681 | CCAGGCCATC ACTTTGCAGC CCGGTGACAT CATCTTCACG |
| 721 | GGAACGCCTG CTGGTGTGGG CCTTGGCCAC AAGCCTCCGC |
| 761 | TGTGGATGAA GGATGGCGAC AAGGTCGAGG TCGAGATCGA |
| 801 | GGGCATCGGT CTGCTCAAGA ACTCGATCGC CAAAGAGGTC |
| 841 | ACTTGA |

A primary consideration when expanding the range of functionalities for consortia into the capacity to degrade harmful chemical compounds is the sensitivity of S. elongatus CscB+ to the toxic and bacteriostatic properties of the harmful chemicals such as dinitrotoluenes (e.g., 2,4-DNT).

Preliminary toxicity assays of planktonic (i.e., free-floating) cultures of S. elongatus cyanobacteria were monitored in the presence of increasing 2,4-DNT. Such S. elongatus cyanobacteria did not contain the enzymatic machinery to breakdown 2,4-DNT. As illustrated in FIG. 13A, when not encapsulated, exponentially growing S. elongatus CscB+ cells were vulnerable to the toxic effects of 2,4-DNT. Growth of S. elongatus was severely inhibited at 2,4-DNT concentrations >0.008 mM (FIG. 13A) and cells exhibited bleaching phenotypes indicative of severe stress and/or cell death at concentrations higher than 0.03 mM (FIG. 13B).

S. elongatus CscB+ cyanobacterial cells were encapsulated in alginate hydrogels as illustrated in FIG. 13C (procedures described in more detail in the foregoing Examples). Results illustrated herein show that the S. elongatus CscB+ cells are viable inside these hydrogels for long time periods (e.g., more than 5 months, see, e.g., FIG. 10). As shown in FIG. 13E, even though these cyanobacterial cells did not express the enzymatic machinery to breakdown 2,4-DNT, such encapsulation enhanced the production of sucrose relative to non-encapsulated planktonic cultures and increased the persistence of these cyanobacteria in the face of nutrient depravation (not shown).

Significantly, encapsulation also improved the resilience of cyanobacterial cells to the toxic effects of 2,4-DNT. Encapsulated S. elongatus CscB+ cells were exposed to increasing concentrations of 2,4-DNT. FIG. 13D-13E illustrates that such encapsulated S. elongatus CscB+ cells remained pigmented, viable, and equally capable of exporting sucrose when exposed to 2,4-DNT concentrations equal to the threshold where P. putida exhibits growth inhibition (0.25 mM). FIG. 13D graphically illustrates that Ba-alginate encapsulated S. elongatus CscB+ cells are resistant to much higher concentrations of 2,4-DNT (at least 0.25 mM) than non-encapsulated S. elongatus CscB+ cells (about 0.03 mM), as evaluated by chlorophyll extraction from beads after 7 days of 2,4-DNT exposure. FIG. 13E graphically illustrates that encapsulated S. elongatus CscB+ cells continue to export sucrose at comparable rates to control encapsulated S. elongatus CscB+ cells that are not exposed to the toxin (measured over 24 hours).

FIG. 13H graphically illustrates the degradation of 2,4-DNT by engineered P. putida strains bearing the heterologous pathway (P. putida-EM-DNT-S) described in FIGS. 13F and 13G. The appearance of pathway intermediates 4-methyl-5-dinitrocatechol (4M5NC) and 2-hydroxy-5-methylquinone (2H5MQ) were tracked by their characteristic absorption spectra (absorption maxima at 420 nm and 485 nm, respectively). FIG. 13I graphically illustrates that the degradation pathway intermediate 4M5NC is only produced by strains bearing the heterologous 2,4-DNT processing pathway, as assayed by liquid chromatography/mass spectrometry. FIG. 13J graphically illustrates that P. putida strains can also produce the bioplastic polymer polyhydroxyalkanoate (PHA). The P. putida-EM-DNT-S can synthesize PHA while also degrading 2,4-DNT.

### Example 11: Indole Manufacture by E. coli-S. elongatus Co-Cultures

This Example illustrates that E. coli strains engineered to overexpress the tryptophanase gene (tnaA) can grow in co-culture with sucrose secreting S. elongatus to produce and secrete the product indole.

A nucleic acid encoding the TnaA tryptophanase enzyme that was used in E. coli is shown below as SEQ ID NO:25.

| | |
|---|---|
| 1 | ATGGAAAACT TTAAACATCT CCCTGAACCG TTCCGCATTC |
| 41 | GTGTTATTGA GCCAGTAAAA CGTACCACTC GCGCTTATCG |
| 81 | TGAAGAGGCA ATTATTAAAT CCGGTATGAA CCCGTTCCTG |
| 121 | CTGGATAGCG AAGATGTTTT TATCGATTTA CTGACCGACA |
| 161 | GCGGCACCGG GGCGGTGACG CAGAGCATGC AGGCTGCGAT |
| 201 | GATGCGCGGC GACGAAGCCT ACAGCGGCAG TCGTAGCTAC |
| 241 | TATGCGTTAG CCGAGTCAGT GAAAAATATC TTCGGTTATC |
| 281 | AATACACCAT TCCGACTCAC CAGGGCCGTG GCGCAGAGCA |
| 321 | AATCTATATT CCGGTACTGA TTAAAAAACG CGAGCAGGAA |
| 361 | AAAGGCCTGG ATCGCAGCAA AATGGTGGCG TTCTCTAACT |
| 401 | ATTTCTTTGA TACCACGCAG GGCCATAGCC AGATCAACGG |
| 441 | CTGTACCGTG CGTAACGTCT ATATCAAAGA AGCCTTCGAT |
| 481 | ACGGGCGTGC GTTACGACTT TAAAGGCAAC TTTGACCTTG |
| 521 | AGGGATTAGA ACGCGGTATT GAAGAAGTTG GTCCGAATAA |
| 561 | CGTGCCGTAT ATCGTTGCAA CCATCACCAG TAACTCTGCA |
| 601 | GGTGGTCAGC CGGTTTCACT GGCAAACTTA AAAGCGATGT |
| 641 | ACAGCATCGC GAAGAAATAC GATATTCCGG TGGTAATGGA |
| 681 | CTCCGCGCGC TTTGCTGAAA ACGCCTATTT CATTAAGCAG |
| 721 | CGTGAAGCAG AATACAAAGA CTGGACCATC GAGCAGATCA |
| 761 | CCCGCGAAAC CTACAAATAT GCCGATATGC TGGCGATGTC |
| 801 | CGCCAAGAAA GATGCGATGG TGCCGATGGG CGGCCTGCTG |
| 841 | TGCATGAAAG ACGACAGCTT CTTTGATGTG TACACCGAGT |
| 881 | GCAGAACCCT TTGCGTGGTG CAGGAAGGCT TCCCGACATA |
| 921 | TGGCGGCCTA GAAGGCGGCG CGATGGAGCG TCTGGCGGTA |
| 961 | GGTCTGTATG ACGGCATGAA TCTCGACTGG CTGGCTTATC |

The tryptophanase with SEQ ID NO:14, is encoded by this nucleic acid segment with SEQ ID NO:25, and was expressed in the E. coli-S. elongatus co-culture.

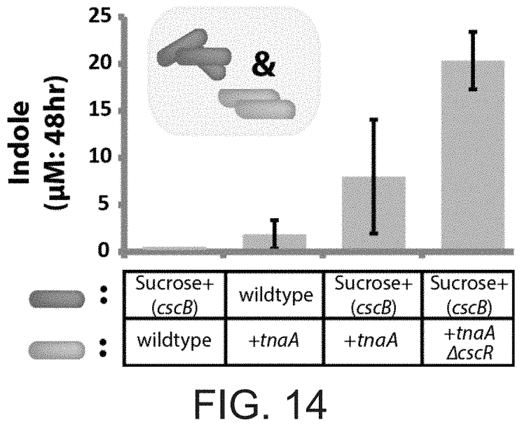

As shown in FIG. 14, the E. coli strain engineered to overexpress this tryptophanase enzyme that was grown in co-culture with sucrose secreting S. elongatus secretes substantial amounts of the product indole. Detectable levels of indole were only measured in cultures with E. coli modified to over-express tnaA. Higher levels of indole are produced in cultures with sucrose-secreting S. elongatus (Sucrose+ cscB) relative to the wildtype. The highest levels of indole are achieved when E. coli is also modified to more efficiently utilize sucrose through the deletion of the sucrose catabolism repressor protein, CscR.

### REFERENCES

1. Hays S G, Ducat D C. Engineering cyanobacteria as photosynthetic feedstock factories. Photosynth Res. 2015; 123:285-95.
2. Möllers K B, Cannella D, Jørgensen H, Frigaard N-U. Cyanobacterial biomass as carbohydrate and nutrient feedstock for bioethanol production by yeast fermentation. Biotechnol Biofuels. 2014; 7:64.
3. Aikawa S, Joseph A, Yamada R, Izumi Y, Yamagishi T, Matsuda F. Kawai H., Chang J-S, Hasunuma T, Kondo A, Ducat D C, Way J C, Silver P A, Lopez P J, Desclés J, Allen A E, Bowler C, Rosenberg J N, Oyler G A, Wilkinson L, Betenbaugh M J, Melis A, Dismukes G C, Carrieri D, Bennette N, Ananyev G M, Posewitz M C, Harun R, Jason WSY, Cherrington T, et al. Direct conversion of Spirulina to ethanol without pretreatment or enzymatic hydrolysis processes. Energy Environ Sci. 2013; 6:1844.
4. Markou H, Angelidaki I, Georgakakis D. Carbohydrate-enriched cyanobacterial biomass as feedstock for bio-methane production through anaerobic digestion. Fuel. 2013; 111:872-9.
5. John R P, Anisha G S, Nampoothiri K M, Pandey A. Micro and macroalgal biomass: A renewable source for bioethanol. Bioresour Technol. 2011; 102:186-93.
6. Rosgaarda L, Jara de Porcellinisa A, Jacobsene J H, Frigaarde N U, Sakuragia
7. Y. Bioengineering of carbon fixation, biofuels, and biochemicals in cyanobacteria and plants. J Biotechnol. 2012; 162(1):134-47.
8. Song K, Tan X, Liang Y, Lu X. The potential of Synechococcus elongatus UTEX 2973 for sugar feedstock production. Appl Microbiol Biotechnol. 2016 100:7865-75.
9. Du W, Liang F, Duan Y, Tan X, Lu X. Exploring the photosynthetic production capacity of sucrose by cyanobacteria. Metab Eng. 2013; 19:17-25.
10. Duan Y, Luo Q, Liang F, Lu X. Sucrose secreted by the engineered cyanobacterium and its fermentability. J Ocean Univ China. 201615:890-6.
11. Xu Y, Guerra L T, Li Z, Ludwig M, Dismukes G C, Bryant D A. Altered carbohydrate metabolism in glycogen synthase mutants of
12. Synechococcus sp. strain PCC 7002: Cell factories for soluble sugars. Metab Eng. 2013; 16:56-67.
13. Niederholtmeyer H, Wolfstädter B T, Savage D F, Silver P A, Way J C. Engineering cyanobacteria to synthesize and export hydrophilic products. Appl Environ Microbiol. 2010; 76:3462-6.
14. Ducat D C, Avelar-Rivas J A, Way J C, Silver P A. Rerouting carbon flux to enhance photosynthetic productivity. Appl Environ Microbiol. 2012; 78: 2660-8.
15. Aikens J, Turner R I. Transgenic photosynthetic microorganisms and photobioreactor. U.S. Pat. No. 8,367,379 B2. Grant, USA. 2013.
16. Aikens J, Turner R J. Method of producing a fermentable sugar. U.S. Pat. No. 8,597,914 B2. Grant, USA. 2013.
17. Chisti Y. Constraints to commercialization of algal fuels. J Biotechnol. 2013; 167:201-14.
18. Bux F, Chisti Y (Eds): Algae Biotechnology. Chain: Springer International Publishing; 2016. [Green Energy and Technology]
19. Singh J. Gu S. Commercialization potential of microalgae for biofuels production. Renew Sustain Energy Rev. 2010; 14:2596-610.
20. Ortiz-Marquez J C F, Do Nascimento M, Zehr J P, Curatti L. Genetic engineering of multispecies microbial cell factories as an alternative for bioenergy production. Trends Biotechnol. 2013:31:521-9.
21. Klähn S, Hagemann M. Compatible solute biosynthesis in cyanobacteria. Environ Microbiol. 2011; 13:551-62.
22. Bockmann J, Heuel H, Lengeler J W. Characterization of a chromosomally encoded, non-PTS metabolic pathway for sucrose utilization in Escherichia coli EC3132. MGG Mol Gen Genet. 1992; 235:22-32.
23. Song H-S, Renslow R S, Fredrickson J K, Lindemann S R. Integrating Ecological and Engineering Concepts of Resilience in Microbial Communities. Front Microbiol. 2015:6:1298.
24. Kim H J, Boedicker J Q, Choi J W, Ismagilov R F. Defined spatial structure stabilizes a synthetic multispecies bacterial community. Proc Natl Acad Sci. 2008; 105:18188-93.
25. Winternute E H, Silver P A. Dynamics in the mixed microbial concourse. Genes Dev. 2010; 24:2603-14.
26. Mee M T, Collins J J, Church G M, Wang H H. Syntrophic exchange in synthetic microbial communities. Proc Natl Acad Sci USA. 2014; 111:E2149-56.
27. Koschwanez J H, Foster K R, Murray A W. Improved use of a public good selects for the evolution of undifferentiated multicellularity. Elife. 2013; 2013:1-27.
28. Koschwanez J H, Foster K R, Murray A W, Keller L. Sucrose Utilization in Budding Yeast as a Model for the Origin of Undifferentiated Multicellularity. PLoS Biol. 2011; 9(8):e1001122.
29. Archer C T, Kim J F, Jeong H, Park J, Vickers C E, Lee S, Nielsen L K. The genome sequence of E. coli W (ATCC 9637): comparative genome analysis and an improved genome-scale reconstruction of E. coli. BMC Genomics. 2011; 12:9.
30. Sabri S, Nielsen L K. Vickers C E. Molecular control of sucrose utilization in Escherichia coli W, an efficient sucrose-utilizing strain. Appl Environ Microbiol. 2013; 79:478-87.
31. Arifin Y, Sabri S, Sugiarto I, Kr6merJO, Vickers C E, Nielsen L K. Deletion of cscR in Escherichia coli W improves growth and poly-3-hydroxybutyrate (PUB) production from sucrose in fed batch culture.J Biotechnol. 56:275-8.
32. Villa F, Pitts B, Lauchnor E, Cappitelli F, Stewart P S. Development of a laboratory model of a phototroph-heterotroph mixed-species biofilm at the stone/air interface. Front Microbiol. 2015:6:1-14.
33. Rossi F, De Philippis R. Role of Cyanobacterial Exopolysaccharides in Phototrophic Biofilms and in Complex Microbial Mats. Life. 2015; 5:1218-38.
34. Lucker B F, Hall C C, Zegarac R, Kramer D M. The environmental photobioreactor (ePBR): An algal culturing platform for simulating dynamic natural environments. Algal Res. 2014; 6(PB):242-9.
35. van Dijl J M, Hecker M. Bacillus subtilis: from soil bacterium to super-secreting cell factory. Microb Cell Fact. 2013; 12:3.
36. Green D M, Colarusso U. The physical and genetic characterization of a transformable enzyme: Bacillus subtilis a-amylase. Biochim Biophys Acta-Spec Sect Enzymol Subj. 1964; 89:277-90.
37. Peoples O P, Sinskey A J. Poly-p-hydroxybutyrate (PHB) biosynthesis in Alcaligenes eutrophus H16. Identification and characterization of the PHB polymerase gene (phbC). J Biol Chem. 1989; 264:15298-303.
38. Song H, Ding M-Z, Jia X-Q. Ma Q Yuan Y-J. Synthetic microbial consortia: from systematic analysis to construction and applications. Chem Soc Rev Chem Soc Rev. 2014; 6954:6954-81.
39. Boehme De, Vincent K, Brown Or. Oxygen and toxicity inhibition of amino acid biosynthesis. Nature. 1976; 262:418-20.
40. Imlay J A. The molecular mechanisms and physiological consequences of oxidative stress: lessons from a model bacterium. Nat Rev Microbiol. 2013; 11:443-54.
41. Gregory E M, Fridovich 1. Oxygen Toxicity and the Superoxide Dismutase. J Bacteriol. 1973; 114:1193-7.
42. Das P K, Bagchi S N. Bentazone and bromoxynil induce H+ and H₂O₂ accumulation, and inhibit photosynthetic 02 evolution in Synechococcous elongatus PCC7942. Pestic Biochem Physiol. 2010; 97:256-61.
43. Morris J J, Kirkegaard R, Szul M J, Johnson Z I, Zinser E R. Facilitation of robust growth of Prochlorococcus colonies and dilute liquid cultures by “helper” heterotrophic bacteria. Appl Environ Microbiol. 2008; 74:4530-4.
44. Beliaev A S, Romine M F, Serres M, Bernstein H C, Linggi B E, Markillie L M, Isern N G, Chrisler W B, Kucek L A, Hill E A, Pinchuk G E, Bryant D A, Wiley S, Fredrickson J K, Konopka A. Inference of interactions in cyanobacterial-heterotrophic co-cultures via transcriptome sequencing. ISME J. 2014; 869: 2243-55.
45. Venn A A, Loram J E, Douglas A E. Photosynthetic symbioses in animals. J Exp Bot. 2008; 59:1069-80.
46. Kihara S, Hartzler D A, Savikhin S. Oxygen Concentration Inside a Functioning Photosynthetic Cell. Biophys J. 2014; 106:1882-9.
47. Leflaive J, Ten-Hage L. Algal and cyanobacterial secondary metabolites in freshwaters: a comparison of allelopathic compounds and toxins. Freshw Biol. 2007; 52:199-214.
48. Do Nascimento M, Dublan M de los A, Ortiz-Marquez J C F, Curatti L. High lipid productivity of an Ankistrodesmus-Rhizobium artificial consortium. Bioresour Technol. 2013; 146:400-7.
49. Bernstein H C, Carlson R P. Microbial Consortia Engineering for Cellular Factories: in vitro to in silico systems. Comput Struct Biotechnol J. 2012; 3, e201210017.
50. Hays S G, Patrick W G, Ziesack M, Oxman N, Silver P A. Better together: Engineering and application of microbial symbioses. Curr Opin Biotechnol. 2015:36:40-9.
51. Balagadd6 F K, Song H, Ozaki J, Collins C H, Barnet M, Arnold F H, Quake S R, You L. A synthetic Escherichia coli predator-prey ecosystem. Mol Syst Biol. 2008; 4:187.
52. Yurtsev E A, Conwill A, Gore J. Oscillatory dynamics in a bacterial cross-protection mutualism. Proc Natl Acad Sci USA. 2016; 113:6236-41.
53. Gore J. Youk H, van Oudenaarden A. Snowdrift game dynamics and facultative cheating in yeast. Nature. 2009; 459:253-6.
54. Chen Y, Kim J K, Hirning A J, Josid K, Bennett M R. Emergent genetic oscillations in a synthetic microbial consortium. Science. 2015; 80-:349.
55. Basu S, Gerchman Y. Collins C H, Arnold F H, Weiss R. A synthetic multicellular system for programmed pattern formation. Nature. 2005.
56. Shou W, Ram S. Vilar JMG: Synthetic cooperation in engineered yeast populations. Proc NatI Acad Sci USA. 2007; 104(6):1877-82.
57. Tamsir A. Tabor J J, Voigt C A. Robust multicellular computing using genetically encoded NOR gates and chemical “wires.”. Nature. 2011; 469:212-5.
58. Scott S R, Hasty J. Quorum Sensing Communication Modules for Microbial Consortia. ACS Synth Biol. 2016; 5(9):969-7⁷.
59. Wintermute E H, Silver P A. Emergent cooperation in microbial metabolism. Mol Syst Biol. 2010:6:407.
60. Mee NIT, Collins J J, Church G M, Wang H-H. Syntrophic exchange in synthetic microbial communities. Proc Natl Acad Sci. 2014; 20.
61. Moffitt J Rl, Lee J B, Cluzel P. The single-cell chemostat: an agarose-based, microfluidic device for high-throughput, single-cell studies of bacteria and bacterial communities. Lab Chip. 2012; 12(8):1487-94.
62. Kim H J, Boedicker J Q, Choi J W, Ismagilov R F. Defined spatial structure stabilizes a synthetic multispecies bacterial community. Proc Natl Acad Sci U.S.A. 2008; 105:18188-93.
63. Smith M J, Francis M B: A Designed A. vinelandii-S. elongatus Coculture for Chemical Photoproduction from Air, Water, Phosphate, and Trace Metals. ACS Synth Biol. 2016; 5(9):955-61.
64. Rai A N, Bergman B, Rasmussen U. Cyanobacteria in Symbiosis. Dordrecht: Springer Netherlands; 2002.
65. Martínez-Pérez C, Mohr W, Löscher C R, Dekaezemacker J, Littmann S, Yilmaz P, Lehnen N, Fuchs B M, Lavik G, Schmitz R A, LaRoche J. Kuypers M M M. The small unicellular diazotrophic symbiont, UCYN-A. is a key player in the marine nitrogen cycle. Nat Microbiol. 2016; 1:16163.
66. Aschenbrenner I A, Cernava T. Berg G, Grube M. Understanding microbial multi-species symbioses. Front Microbiol. 2016; 7:180.
67. Hom E F Y, Murray A W. Plant-fungal ecology. Niche engineering demonstrates a latent capacity for fungal-algal mutualism. Science. 2014; 345:94-8.
68. Ramanan R, Kim B-H, Cho D-H, Oh H-M, Kim H-S: Algae-bacteria interactions: Evolution, ecology and emerging applications. Biotechnol Adv. 2016; 34:14-29.
69. Kearns D B, Chu F, Branda S S, Kolter R. Losick R. A master regulator for biofilm formation by Bacillus subtilis. Mol Microbiol. 2005; 55:739-49.
70. Torella J P, Gagliardi C J, Chen J S, Bediako D K, Colón B, Way J C, Silver P A, Nocera D G. Efficient solar-to-fuels production from a hybrid microbial-water-splitting catalyst system. Proc Natl Acad Sci. 2015:112:2337-42.
71. Liu C, Gallagher J J, Sakimoto K K, Nichols E M, Chang C J, Chang MCY, Yang P: Nanowire-Bacteria Hybrids for Unassisted Solar Carbon Dioxide Fixation to Value-Added Chemicals. Nano Lett. 2015; 15(5):3634-9.
72. Datsenko K A, Wanner B L. One-step inactivation of chromosomal genes in Escherichia coli K-12 using PCR products. Proc Natl Acad Sci USA. 2000; 97: 6640-5.
73. Xuan Y H, Hu Y B, Chen L-Q, Sosso D, Ducat D C, Hou B-H, Frommer W B. Functional role of oligomerization for bacterial and plant SWEET sugar transporter family. Proc Natl Acad Sci. 2013; 110(39):E3685-E3694.
74. Abramson B W, Kachel B, Kramer D M, Ducat D C. Increased photochemical efficiency in cyanobacteria via an engineered sucrose sink. Plant Cell Physiol. 2016; 57(12):2451-2460.
75. Nikel P I, Martinez-Garcia E, de Lorenzo V. Biotechnological domestication of pseudomonads using synthetic biology. Nat Rev Microbiol. 2014; 12(5):368-379.
76. Nielsen A Z. Mellor S B, Vavitsas K, et al. Extending the biosynthetic repertoires of cyanobacteria and chloroplasts. Plant J. 2016; 87(1):87-102.
77. Waclawovsky A J, Sato P M, Lembke C G, Moore P H, Souza G M. Sugarcane for bioenergy production: An assessment of yield and regulation of sucrose content. Plant Biotechnol J. 2010; 8(3):263-276.
78. Richardson J W, Outlaw J L, Allison N. The economics of microalgae oil. AgBioForum. 2010; 13(2):119-130.
79. Lee R A, Lavoie J-M. From first- to third-generation biofuels: Challenges of producing a commodity from a biomass of increasing complexity. Anim Front. 2013; 3(2):6-11.
80. Eisentraut A. Sustainable Production of Second-generation Biofules—. Renew Energy. 2010:1-39.
81. Runge C F, Senauer B. Kill king corn. Nature. 2007; 449(7163):637.
82. Williams P R D, Inman D, Aden A, Heath G A. Environmental and sustainability factors associated with next-generation biofuels in the U.S.: What do we really know? Environ Sci Technol. 2009; 43(13):4763-4775.
83. Carpenter D, Westover T L, Czernik S. Jablonski W. Biomass feedstocks for renewable fuel production: a review of the impacts of feedstock and pretreatment on the yield and product distribution of fast pyrolysis bio-oils and vapors. Green Chem. 2014; 16(2):384-406.
84. Merugu R, Girisham S, Reddy S M. Production Of PHB (Polyhydroxybutyrate) by Rhodopseudomonas Palustris Ku003 and Rhodobacter Capsulatus KU002 Under Phosphate Limitation. Int J Appl Biol Pharm Technol. 2010; 1:847-850.
85. Dismukes G C, Carrieri D, Bennette N, Ananyev G M, Posewitz M C. Aquatic phototrophs: efficient alternatives to land-based crops for biofuels. Curr Opin Biotechnol. 2008; 19(3):235-240.
86. Ducat D C, Way J C, Silver P A. Engineering cyanobacteria to generate high-value products. Trends Biotechnol. 2011; 29(2):95-103.
87. Hondo S, Takahashi M, Osanai T, et al. Genetic engineering and metabolite profiling for overproduction of polyhydroxybutyrate in cyanobacteria. J Biosci Bioeng. 2015; 120(5):510-517.
88. Ansari S, Fatma T. Cyanobacterial polyhydroxybutyrate (P-1B): Screening, optimization and characterization. PLoS One. 2016; 11(6).
89. Wang B, Pugh S, Nielsen D R, Zhang W, Meldrum D R. Engineering cyanobacteria for photosynthetic production of 3-hydroxybutyrate directly from CO₂. Metab Eng. 2013; 16(1):68-77.
90. Tsang T K, Roberson R W, Vermaas WFJ. Polyhydroxybutyrate particles in Synechocystis sp. PCC 6803: Facts and fiction. Photosynth Res. 2013; 118(1-2):37-49.
91. Nishioka M, Nakai K, Miyake M, Asada Y, Taya M. Production of poly-β-hydroxybutyrate by thermophilic cyanobacterium, Synechococcus sp. MA19, under phosphate-limited conditions. Biotechnol Lett. 2001; 23(14):1095-1099.
92. Zhang S, Liu Y, Bryant D A. Metabolic engineering of Synechococcus sp. PCC 7002 to produce poly-3-hydroxybutyrate and poly-3-hydroxybutyrate-co-4-hydroxybutyrate. Metab Eng. 2015; 32:174-183.
93. Criddle, C. S., Billington, S. L., Frank, C. W., 2014. Renewable bioplastics and biocomposites from biogas methane and waste-derived feedstock: Development of enabling technology, life cycle assessment, and analysis of costs, California Department of Resources, Recycling, and Recovery. California Department of Resources Recycling and Recovery, State of California.
94. Cheali, P., Posada, J. A., Gernaey, K. V., Sin, G., 2016. Economic risk analysis and critical comparison of optimal biorefinery concepts. Biofuels, Bioprod. Biorefining 10, 435-445.
95. Ghatak, H. R., 2011. Biorefineries from the perspective of sustainability: Feedstocks, products, and processes. Renew. Sustain. Energy Rev. 15, 4042-4052.
96. Tegtmeier, E. M., Duffy, M. D., 2004. External costs of agricultural production in the United States. Int. J. Agric. Sustain. 2, 1-20.
97. Leong, Y. K., Show, P. L., Ooi, C. W., Ling, T. C., Lan, J. C.-W., 2014. Current trends in polyhydroxyalkanoates (PHAs) biosynthesis: Insights from the recombinant Escherichia coli. J. Biotechnol. 180, 52-65.
98. Reddy, M. M., Vivekanandhan, S., Misra, M., Bhatia, S. K., Mohanty, A. K., 2013. Biobased plastics and bionanocomposites: Current status and future opportunities. Prog. Polym. Sci. 38, 1653-1689.
99. Venkata Mohan, S., Venkateswar Reddy, M., 2013. Optimization of critical factors to enhance polyhydroxyalkanoates (PHA) synthesis by mixed culture using Taguchi design of experimental methodology. Bioresour. Technol. 128, 409-416.
100. Gavrilescu, M., Chisti, Y., 2005. Biotechnology-A sustainable alternative for chemical industry. Biotechnol. Adv. 23, 471-499.
101. Vickers, C. E., Klein-Marcuschamer, D., Krömer, J. O., 2012. Examining the feasibility of bulk commodity production in Escherichia coli. Biotechnol. Lett. 34, 585-596.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A cyanobacteria encapsulated in a hydrogel.
2. The cyanobacteria of statement 1, modified to express a sucrose/proton symporter.
3. The cyanobacteria of statement 1 or 2, comprising an expression vector comprising a nucleic acid segment encoding a sucrose/proton symporter.
4. The cyanobacteria of statement 1, 2, or 3, comprising a heterologous expression vector comprising a promoter operably linked to a nucleic acid segment encoding a sucrose/proton symporter.
5. The cyanobacteria of statement 1-3, or 4, comprising a heterologous expression vector comprising a promoter operably linked to a nucleic acid segment encoding a sucrose/proton symporter, wherein the promoter is operable in a cyanobacteria.
6. The cyanobacteria of statement 1-4, or 5, wherein the sucrose/proton symporter is a bacterial cscB protein.
7. The cyanobacteria of statement 1-5, or 6, wherein the encapsulated cyanobacteria are viable for at least three months, or at least four months, or at least five months, or at least six months.
8. The cyanobacteria of statement 1-6, or 7, wherein the encapsulated cyanobacteria are viable through repeated cycles of nitrogen availability.
9. The cyanobacteria of statement 1-7, or 8, wherein the encapsulated cyanobacteria are stable and viable through extended periods of nitrogen deprivation.
10. The cyanobacteria of statement 1-8, or 9, wherein the encapsulation of the cyanobacteria blocks predation planktonic grazer species.
11. The cyanobacteria of statement 1-9, or 10, wherein the encapsulation of the cyanobacteria blocks infection by cyanobacterial viruses.
12. The cyanobacteria of statement 1-10, or 11, wherein the encapsulation of the cyanobacteria slows cyanobacterial division.
13. The cyanobacteria of statement 1-11, or 12, wherein the encapsulation of the cyanobacteria reduces loss of recombinantly introduced expression cassettes.
14. A consortium comprising a heterotrophic microbial population and an autotrophic cyanobacteria population.
15. The consortium of statement 14, wherein the cyanobacteria population comprises the cyanobacteria of statement 1-12, or 13.
16. The consortium of statement 14 or 15, wherein the cyanobacteria population comprises cyanobacteria that convert carbon dioxide to sugar when the cyanobacteria are exposed to light.
17. The consortium of statement 14, 15 or 16, wherein the heterotrophic microbial population comprises microbes that synthesize a product or metabolize an environmental toxin or metabolize an environmental pollutant.
18. The consortium of statement 14-16 or 17, wherein the heterotrophic microbial population comprises at least one type of bacteria, fungi, algae, or a combination thereof.
19. The consortium of statement 14-17 or 18, wherein the heterotrophic microbial population comprises a strain of bacteria that can utilize sugar produced by the cyanobacteria as a carbon source.
20. The consortium of statement 14-18 or 19, wherein the heterotrophic microbial population comprises at least one type of transgenic microbe.
21. The consortium of statement 14-19 or 20, wherein the heterotrophic microbial population comprises microbes that transgenically express one or more types of enzymes.
22. The consortium of statement 14-20 or 21, wherein the heterotrophic microbial population comprises microbes that transgenically express one or more types of enzymes that can manufacture polyhydroxybutyrate (PHB)
23. The consortium of statement 14-21 or 22, wherein the heterotrophic microbial population comprises microbes that transgenically express one or more types of enzymes that metabolize aromatic toxins.
24. The consortium of statement 14-22 or 23, wherein the heterotrophic microbial population comprises microbes that transgenically express one or more types of enzymes that can manufacture indole.
25. The consortium of statement 14-23 or 24, wherein the hydrogel comprises alginate, latex, silica, or a combination thereof.
26. The consortium of statement 14-24 or 25, wherein the hydrogel encapsulation allows diffusion of gases, nutrients, and other molecules to and from the cyanobacteria.
27. A method comprising culturing a consortium comprising a cyanobacteria population and a heterotrophic microbial population in a culture medium for a time and under conditions sufficient for the consortium to produce a product, and isolating the product from the culture medium, the heterotrophic microbial population, or the cyanobacteria.
28. The method of statement 27, further comprising sparging the culture medium with a low oxygen gas to reduce oxygen accumulation, or culturing in a culture medium containing DCMU (3-(3,4-dichlorophenyl)-1,1-dimethylurea) or thiosulfate.
29. The method of statement 27 or 28, wherein the cyanobacteria population comprises the cyanobacteria of statement 1-12, or 13.
30. The method of statement 27, 28 or 29, wherein the consortium is the consortium of statement 14-24 or 25.
31. The method of statement 27-29 or 30, wherein the heterotrophic microbial population comprises bacteria, fungi, algae, or a combination thereof.
32. The method of statement 27-30 or 31, wherein the heterotrophic microbial population comprises engineered (e.g., recombinant) bacteria, fungi, algae, or a combination thereof.
33. The method of statement 27-31 or 32, wherein the heterotrophic microbial population comprises one or more strains of E. coli, Bacillus subtilis, Halomonas boliviensis, Saccharomyces cerevisiae, Pseudomonas putida, or a combination thereof.
34. The method of statement 27-32 or 33, wherein the product is at least one drug, enzyme, nutrient, protein, oil, carbohydrate, alcohol, fatty acid, vitamin, pigment, pharmaceutical enzyme, biotechnological enzyme, gas (e.g., hydrogen), polymer substrate, polymer monomer, polymer, biofuel, metabolite, and combinations thereof.
35. The method of statement 27-33 or 34, wherein the product is alpha-amylase, or hydroxylated alkyl acid.
36. The method of statement 27-34 or 35, wherein the product is polyhydroxybutyrate or indole.
37. The method of statement 27-35 or 36, wherein the product is biomass.
38. A method comprising applying the consortium of statement 14-25 or 26 to a site for a time and under conditions sufficient for the consortium to metabolize an environmental toxin and/or metabolize an environmental pollutant, and to thereby reduce the toxin or pollutant concentration at the site.
39. The method of statement 38, wherein the consortium comprises a population of encapsulated cyanobacteria and a population of heterotrophic microbes engineered to express enzymes that metabolize the environmental toxin or metabolize the environmental pollutant.
40. The method of statement 38 or 39, wherein the consortium comprises a population of encapsulated cyanobacteria and Pseudomonas putida engineered to express enzymes that metabolize aromatic toxins.

The specific products, consortia, methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential.

The specific products, consortia, methods and compositions illustratively described herein suitably may be practiced in differing orders of steps, and the methods and processes are not necessarily restricted to the orders of steps indicated herein or in the claims. As used herein and in the appended claims, the singular forms “a,” “an,” and “the” include plural reference unless the context clearly dictates otherwise. Thus, for example, a reference to “a microbe,” “a compound,” “a nucleic acid” or “a promoter” includes a plurality of such microbes, compounds, nucleic acids or promoters (for example, a solution of microbes, compounds or nucleic acids, or a series of promoters), and so forth.

The term “about” can allow for a degree of variability in a value or range, for example, within 10%, within 5%, or within 1% of a stated value, or of a stated limit of a range. When a range or a list of sequential values is given, unless otherwise specified any value within the range or any value between the given sequential values is also disclosed.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

What is claimed:

1. A consortium comprising a population of heterotrophic microbes and a population of the cyanobacterial cells, where the cyanobacterial cells are modified to express a sucrose/proton symporter, and where the cyanobacterial cells are encapsulated in a hydrogel.

2. The consortium of claim 1, wherein the cyanobacteria are encapsulated.

3. The consortium of claim 1, wherein the cyanobacteria are encapsulated into hydrogel beads.

4. The consortium of claim 1, wherein the cyanobacteria are Synechocystis, Synechococcus, Thermosynechococcus, Nostoc, Prochlorococcu, Microcystis, Anabaena, Spirulina, or Gloeobacter cyanobacteria.

5. The consortium of claim 1, wherein the heterotrophic microbes comprise at least one type of bacteria, fungi, algae, or a combination thereof.

6. The consortium of claim 1, wherein at least one type of the heterotrophic microbes comprises a transgene or expression cassette encoding a heterologous enzyme.

7. The consortium of claim 1, wherein at least one type of the heterotrophic microbes expresses a heterologous enzyme.

8. The consortium of claim 1, wherein the heterotrophic microbes comprise microbes that synthesize a product or metabolize an environmental toxin or metabolize an environmental pollutant.

9. The consortium of claim 1, wherein the heterotrophic microbes comprise a strain of bacteria that can utilize sugar produced by the cyanobacteria as a carbon source.

10. The consortium of claim 1, wherein the heterotrophic microbes transgenically express one or more types of enzymes.

11. The consortium of claim 1, wherein the heterotrophic microbes comprise microbes that transgenically express one or more types of enzymes that can manufacture polyhydroxybutyrate (PHB).

12. The consortium of claim 1, wherein the heterotrophic microbes comprise microbes that transgenically express one or more types of enzymes that metabolize aromatic toxins.

13. The consortium of claim 1, wherein the heterotrophic microbes comprise microbes that transgenically express one or more types of enzymes that can manufacture indole.

14. A method comprising culturing the consortium of claim 1 under conditions that promote the growth of the heterotrophic microbes.

15. The method of claim 14, further comprising culturing the consortium under conditions that promote the synthesis of a product by heterotrophic microbes.

16. The method of claim 14, further comprising harvesting the product.

17. The method of claim 14, wherein the cyanobacteria are encapsulated into beads of alginate.

18. The method of claim 14, wherein the cyanobacteria are encapsulated by a method comprising dripping a cyanobacteria cell/alginate suspension into a barium chloride curing solution to produce a population of encapsulated cyanobacteria.

19. A method comprising applying the consortium of claim 1 to a site for a time and under conditions sufficient for the consortium to metabolize an environmental toxin and/or metabolize an environmental pollutant, to thereby reduce the toxin or pollutant concentration at the site.

20. The method of claim 19, wherein the conditions promote the growth of the heterotrophic microbes.

21. The method of claim 19, wherein at least one type of the heterotrophic microbes comprises a transgene or expression cassette encoding a heterologous enzyme.

22. The method of claim 19, wherein the heterotrophic microbes comprise microbes that transgenically express one or more types of enzymes that can manufacture polyhydroxybutyrate (PHB).

23. The method of claim 19, wherein the heterotrophic microbes comprise microbes that transgenically express one or more types of enzymes that metabolize aromatic toxins.

24. The method of claim 19, wherein the heterotrophic microbes comprise microbes that transgenically express one or more types of enzymes that can manufacture indole.


SEQUENCE LISTING (25 sequences)

SEQ ID NO: 1 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKGHQCVAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 2 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctaggattaacagggacggaattaggtacactttattcggtcaaccagtttaccagcattctatttatgatgttctacggcatcgttcaggataaactcggtctgaagaaaccgctcatctggtgtatgagtttcattctggtcttgaccggaccgtttatgatttacgtttatgaaccgttactgcaaagcaatttttctgtaggtctaattctgggggcgctcttttttggcctggggtatctggcgggatgtggtttgcttgacagcttcactgaaaaaatggcgcgaaattttcatttcgaatatggaacagcgcgcgcctggggatcttttggctatgctattggcgcgttctttgccggcatattttttagtatcagtccccatatcaacttctggctggtctcgctatttggcgctgtatttatgatgatcaacatgcgttttaaagataagggtcaccagtgtgtagcggcggatgcgggaggggtaaaaaaagaggattttatcgcagttttcaaggatcgaaacttctgggtttttgtcatatttattgtggggacgtggtctttctataacatttttgatcaacaactctttcctgtcttttatgcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggtgttgtgattatggcgttgcgtatcctttcctgcgcgttgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatag

SEQ ID NO: 3 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCIAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 4 - DNA, 1369 bp, Escherichia coli
gtcgacaagaactggaaagtgagaagtaaacggcgaggccgctcttatctctaaataggacatgaattttttaacgacaggcaggtaattatggcactgaatattccattcagaaatgcgtactatcgttttgcatccagttactcatttctcttttttatttcctggtcgctgtggtggtcgttatacgctatttggctgaaaggacatctaggattaacagggacggaattaggtacactttattcggtcaaccagtttaccagcattctatttatgatgttctacggcatcgttcaggataaactcggtctgaagaaaccgctcatctggtgtatgagtttcattctggtcttgaccggaccgtttatgatttacgtttatgaaccgttactgcaaagcaatttttctgtaggtctaattctgggggcgctcttttttggcctggggtatctggcgggatgcggtttgcttgacagcttcaccgaaaaaatggcgcgaaattttcatttcgaatatggaacagcgcgcgcctggggatcttttggctatgctattggcgcgttctttgccggtatattttttagtatcagtccccatatcaacttctggttggtctcgctatttggcgctgtatttatgatgatcaacatgcgttttaaagataaggatcaccagtgcatagcggcggatgcgggaggggtaaaaaaagaggattttatcgcagttttcaaggatcgaaacttctgggttttcgtcatatttattgtggggacgtggtctttctataacatttttgatcaacaactctttcctgtcttttatgcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggtgttgtgattatggcgttgcgtatcctttcctgcgcgttgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatagacgtaaactttttccggttgttgtcgatatc

SEQ ID NO: 5 - protein, 447 aa, Uncultured bacterium
MIMASATKSAWKNPSYLQSSFGIFMFFCSWGIWWSFFQRWLISGVGLTNAEVGTIYSINSLATLVIMFVYGVIQDQLGIKRKLVIVVSVIAACVGPFVQFVYAPMILAGGTTRWIGALIGSIVLSAGFMSGCSLFEAVTERYSRKFGFEYGQSRAWGSFGYAIVALCAGFLFNINPLINFWVGSAFGLGMLLVYAFWVPAEQKEELKKETDPNAAPTNPSLKEMVAVLKMPTLWVLIVFMLLTNTFYTVFDQQMFPTYYANLFPTEEIGNATYGTLNGFQVFLESAMMGVVPIIMKKIGVRNALLLGATVMFLRIGLCGVFHDPITISIVKLFHSIEVPLFCLPAFRYFTLHFDTKLSATLYMVGFQIASQVGQVIFSTPLGAFHDKMAQILPNNDMGSRVTFWVISAIVLCALIYGFFVIKRDDQEVGGDPFYTDKQLRQMEAAKA

SEQ ID NO: 6 - protein, 352 aa, Escherichia coli
LDLASDVCFCYSARFTANGLGMASLKDVARLAGVSMMTVSRVMHNAESVRPATRDRVLQAIQTLNYVPDLSARKMRAQGRKPSTLAVLAQDTATTPFSVDILLAIEQTASEFGWNSFLINIFSEDDAARAARQLLAHRPDGIIYTTMGLRHITLPESLYGENIVLANCVADDPALPSYIPDDYTAQYESTQHLLAAGYRQPLCFWLPESALATGYRRQGFEQAWRDAGRDLAEVKQFHMATGDDHYTDLASLLNAHFKPGKPDFDVLICGNDRAAFVAYQVLLAKGVRIPQDVAVMGFDNLVGVGHLFLPPLTTIQLPHDIIGREAALHIIEGREGGRVTRIPCPLLIRCST

SEQ ID NO: 7 - DNA, 1059 bp, Escherichia coli
tcaggtggaacaacggatcaacagcgggcaagggatccgcgtcactcttcccccttcacgaccttcaataatatgcaatgcagcttcccgcccgataatgtcatgtggaagctgaattgtggtcagcggcggtaaaaacagatgcccgacgccaaccagattatcaaagcccattacggcgacatcctgcgggattcgtacccccttcgccagaagaacctgataagccacaaaggctgcgcgatcgttaccacatatcagaacatcaaaatctggtttgcccggtttgaagtgggcattgagtaaacttgcgagatcggtgtagtgatcatcacctgttgccatgtgaaattgtttcacctcagccagatctcgtccagcatcacgccaggcctgctcaaatccctgccgacgataccctgttgccaacgcactttccggtagccagaagcataacggttgacgatagcccgccgcgagcaaatgctgtgttgattcatattgtgcagtgtaatcatcagggatataactgggtaacgctgggtcatccgccacacagttcgccaatacaatattttcaccatacagagactcaggcagcgtgatatgtcgcagccccattgtagtatagataatgccatccggacggtgggcaagcagctgacgtgccgcgcgggcagcgtcatcttcagaaaaaatattgattaaaaaactattccagccgaactcgctggcggtttgctcaatggcaagcagaatatcaacagagaaaggagtggtagccgtgtcctgcgccagcacggcgagagtcgacggcttacgtccttgagcgcgcatcttacgggcggaaagatcaggaacataattcagggtctggattgcctgcaatacgcggtcacgcgttgcaggacgcacagattctgcattatgcatcacccgggagactgtcatcatcgacactcccgccaggcgtgcgacatcctttaatgaagccatacccaagccgtttgccgtaaaacgggcactgtagcagaaacagacgtcactggcgagatccaa

SEQ ID NO: 8 - protein, 392 aa, Zoogloea ramigera
MSTPSIVIASARTAVGSFNGAFANTPAHELGATVISAVLERAGVAAGEVNEVILGQVLPAGEGQNPARQAAMKAGVPQEATAWGMNQLCGSGLRAVALGMQQIATGDASIIVAGGMESMSMAPHCAHLAGGVKMGDFKMIDTMIKDGLTDAFYGYHMGTTAENVAKQWQLSRDEQDAFAVASQNKAEAAQKDGRFKDEIVPFIVKGRKGDITVDADEYIRHGATLDSMAKLRPAFDKEGTVTAGNASGLNDGAAAALLMSEAEASRRGIQPLGRIVSWATVGVDPKVMGTGPIPASRKALERAGWKIGDLDLVEANEAFAAQACAVNKDLGWDPSIVNVNGGAIAIGHPIGASGARILNTLLFEMKRRGARKGLATLCIGGGMGVAMCIESL

SEQ ID NO: 9 - protein, 246 aa, Ralstonia eutropha
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 10 - protein, 589 aa, Cupriavidus necator
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 11 - protein, 104 aa, Burkholderia sp
MSENWIDAAARDEVPEGDVIGINIVGKEIALYEVAGEIYATDNTCTHGAARMSDGFLEGREIECPLHQGRFDVCTGKALCTPLTQDIKTYPVKIENMRVMLKLD

SEQ ID NO: 12 - protein, 161 aa, Burkholderia sp
MVDFKTYFELLNLYSDYAMVCDSANWEKWPDFFIETGTYRLQPRENFEQDLPLCLLALESKAMIRDRVYGVKETMYHDPYYQRHIVGTPRVLSVERDADGERITAEASYAVIRTKYDGDSTIFNAGYYRDVIVRTPEGLKLKSRLCVYDSEMIPNSIIYPI

SEQ ID NO: 13 - protein, 276 aa, Burkholderia sp
MAEIVAGFMLPHDPLIASIPDAPPLQKRETCMAAYAAIVERIKDLKVDTVIVIGDDHYTMHSPACIPRCLIGIGDVEGPREEWLGIPRAKIENNEALAHHIMQTGFDVGVDWAVAKTLVIDHSTTIPIHYAVRPAAGVRAIPVYLNTGFEPLISSRRAYQIGKVIGEAVASWSGTERVAIYGTGGLSHWPGMAQMGKVNAEWDRKILSHVEEGNVEALIALTDEEILRDGGNGGLEIKNWICAMGALGTVRGELIAYESVPEWVCGCGYLEMKRAA

SEQ ID NO: 14 - protein, 333 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAY

SEQ ID NO: 15 - protein, 460 aa, Sediminispirochaeta smaragdinae
MAITYLPEPFRIKMVETIKMLTPEEREQRIAEAKYNLFNLKGKDVYIDLLTDSGTNAMSQEQWAGVMKGDEAYAGGASYYKLLEAAQDITGYAYIQPVHQGRAAEKVLFGILMGKGQFSISNMHFDTTRAHVELTGARAIDCVVSDAMDPAKRAPFKGNMDVDKLESLIKKYGAEKVALVVMTVTNNSAGGQPVSMQNIRETASVCKKYNIKFCIDAARFAENAFFIKKREDGYRDASIKDITNEMFSYADMFTMSAKKDAIVNMGGLIGIKDDAELFSACKGRTISFEGFITYGGLSGRDLESLAIGLYEGIDENQLRYRIGQIEYLASRLDDAGITYQAPAGGHGIFVDAKALLPHIPYYEFPGQALAVELYKEAGIRTCDIGSYMLGNDPDTGKQLHAEFEFTRLAIPRRVYTQAHIDVMAEALIEIKKRANRVKGYKITWEPPVLRHFQASLKPLT

SEQ ID NO: 16 - DNA, 906 bp, Pseudomonas putida
atggatctgcgcgacatcgacttgaatctgctggtggtcttcaaccagctactgctcgaccggagcgtatcgacggccggcgaaaaactggggctgacgcagcctgccgtcagtaattcacttaaacggctgcgtacagcgctgaacgacgatttgttcttgcgcacctcaaaaggcatggagccgacaccgtatgcactgcatcttgcggagcccgtgatctatgcgctcaacacgctgcagacggcactgacgacccgtgactctttcgacccatttgccagcacgcgcaccttcaacttggcaatgaccgacatcggcgagatgtacttcatgcccccactgatggaagcgcttgcgcaacgagctcctcacatccagatcagcacgctgcgcccgaatgctggcaatctgaaggaggatatggagtccggtgcggttgatctcgccttgggtcttctgccagagctacagaccggattcttccagcggcgcctctttcgccaccgctacgtatgcatgttccgcaaggaccatccaagcgccaaatcccccatgagcctgaaacagttcagtgaactggagcatgtcggcgtggtcgcactcaacaccggacacggtgaggtcgatggcctgctcgaacgcgcaggcatcaaaaggcgcatgcggctggtggtgccgcatttcattgcgatcggccccattctgcacagcaccgacctcatcgcgaccgtgccgcagcgttttgccgttcgctgcgaagtgccttttggtctgacgacatccccgcacccggccaagctgcctgacatcgccatcaacctgttttggcatgccaagtacaaccgggatccgggcaacatgtggctacgtcagttgttcgtcgagcttttctctgaagcataa

SEQ ID NO: 17 - DNA, 987 bp, Pseudomonas putida
atggaactggtagtagaacccctcaatttgcatctgaacgcggagaccggcagcaccctgcttgacgtgctcaggtccaacgaggtccccatttcttatagctgcatgtcgggccgctgcggcacttgccgttgccgtgtgattgccggccatcttcgcgataacggccccgagacagggcgcccgcaggcaggaaagggggcctatgtcctggcctgtcaggcggttctgaccgaagactgcacgatcgagattcctgaatctgacgagatcgtggttcacccggcgcgcatcgtcaaggggacggtcacagcgatagacgaagccacccatgacatccggcgcctgcgcatcaaactggccaaaccgcttgagttcagccctggccagtacgcaacggtgcagttcacgcccgaatgcgtccgcccctattcgatggccgggctgcctagcgatgcggaaatggagtttcagattcgcgcggttccgggcgggcatgtcagcaactacgttttcaatgaactgtccgtaggcgcttcggtgcggatcagcggccccctcggaacggcctatctgcggcgcacgcacaccggccccatgctttgtgtggggggtggaacaggtctggcgcccgtcctttcgatcgttcgaggcgcactggaaagcgggatgagcaaccccatccatctgtacttcggtgtgcggagcgagcaggacatctatgacgaggaacgccttcacgcattggctgcaaggtttccgaatctcaaggtgaatgtcgttgttgcaacaggccctgccggccctggtcatcgatccggcctggtcaccgatctgatcggccgtgacttgcccaatttggcgggatggcgcgcctacctgtgtggcgctccggccatggtcgaggccctgaacctgctcgttgctcgcctaggcatagtacccgggcacatccatgccgatgcgttctatcccagcggcgtctga

SEQ ID NO: 18 - DNA, 315 bp, Pseudomonas putida
atgagcgagaactggatcgacgccgccgcccgcgacgaggtgcccgagggcgacgtgatcggcatcaatatcgtcggcaaggagattgccctctacgaggtggcgggcgagatctacgccaccgacaacacctgcactcacggcgccgcccgcatgagcgatggctttctcgaaggccgggaaattgaatgtcctttgcatcaaggccgattcgatgtttgcacgggtaaagccttgtgcacacccctgacacaggacatcaaaacctaccccgtaaaaatcgaaaacatgcgcgtgatgctcaagctggactaa

SEQ ID NO: 19 - DNA, 1344 bp, Pseudomonas putida
atgagttaccaaaacttagtgagtgaagcagggctgacgcaaaagcacctgatttatggcgacaaagaacttttccagcacgaattgaagaccatcttcgcgcggaactggctttttctgacccatgacagtctgattccctcccccggcgactatgtcaaagccaaaatgggcgtcgatgaagtcatcgtctcccgccagaacgatggctcggtgcgagcctttttgaatgtttgccgtcaccggggcaagacaatagttgacgctgaagccggaaatgcgaaaggctttgtgtgcggttaccacggctggggctatggctccaacggcgaactgcaaagcgttccctttgaaaaagagttgtacggagatgcgatcaaaaagaaatgcctgggcttgaaagaagtcccccgcatcgaaagctttcatggctttatctatggctgttttgatgcagaagctcccccgctcatcgattatctgggtgatgcagcctggtacctggaacccaccttcaagcactctggtggcctggaacttgtaggcccccccggcaaagtggtggttaaggccaactggaagcctcttgcggaaaactttgtaggtgacgtctaccacattggttggacgcacgcatctattttgcgcgcagggcagtcgatatttgctcctcttgcgggcaacgctatgtttccacccgaaggcgcgggcttgcaaatgaccaccaagtatggcagtggaattggcgtattgtgggacgcctactccggtatccagagcgctgatatggttcccgaaatgatggcattcggcggcgcaaaacaggaaaagctcgccaaagaaatcggcgatgtccgggcgcggatttaccgcagccaactgaacggcacggttttcccgaacaacagctttttgacctgctccggtgtcttcaaggtctttaacccgatcgatgaaaacacgaccgaggtttggacgtatgccatcgtagaaaaagacatgcctgaggacttaaagcgtcgcttggctgacgcggttcagcgcagtgtcggaccagcaggatactgggaaagcgacgacaacgacaacatggggacgttgtcgcaaaatgccaagaaataccaatccagcaacagtgatctgattgccgatttgggtttcggcaaggacgtctacggcgacgaatgctatccgggcgtcgttggcaaatcggcaatcagcgaaaccagctatcgcggattctaccgtgcctaccaggctcacatcagcagctccaattgggccgagttcgaaaacacctcccgaaattggcacaccgaactcaccaagacgactgatcgctaa

SEQ ID NO: 20 - DNA, 585 bp, Pseudomonas putida
atgatgatcaatacccaggaagacaagctggtctccgcgcacgacgccgaagaatttcaccgtttcttcgtcgggcacgacagcgatctgcagcaagaagtcaccacactcctgacccgcgaagcgcacctgctggacattcaggcctacaaagcctggcttgaacactgcgttgcccccgagatcaaataccaagtgatctcgcgagaacttcgctccacttccgagcgtcgataccaactgaatgatgcggtgaatatctacaacgagaactatcaacagctgaaagttcgagttgaacaccagatggatcctcagaactggtacaacagcccgaagatccgcttcacccgcttcgtcaccaatgtcacggcggccaaggacaagagcgcaccggaaatgctgcatgtgcggtccaacctcattctccatcgcgccagacgaggaaaccaagttgacgtcttctatgcaacgcgagaagacaaatggaaacgcatcgaaggtggtggcatcaaattggtcgaacgctttgtggactacccggagcgcagtccccaaacccacaacctgataatcttcctgtga

SEQ ID NO: 21 - DNA, 1683 bp, Pseudomonas putida
gtgcatcacgtttctactaagtcgccgtctaccttgtcggcggagtgtgaagttctcatcgtcgggggtagcttggtcggcttgtcgcttgcaaactttctcggccaccacggcgtaagcgctgcagtcgtcgagcggcacaagggaacggccatccaccctcgtgctggccactttcacctgaggaccattgaagcatttcgatatgcaggaatcgagccagaagtcatgcaggagtctcttcgacagttcgatccggacggcggtatcaacgtcgtcgaatcgcttgccggcaaggagatcgccagcctgattggcaacttgaacgaaggcgtcgaaaaactcagtccaagtaagcgcctgttcatgacacaacaaagtctcgaacccttgcttagaaagaacgctgaaaagctgggggcccaacttaactaccagatggaattggtttcattcgagcaggacgccacgggtgtgactgcgcgagtcaggtacatcccatccggggccgtgagccaagtgcgtgccaagtacctaatcgccgccgacggtaaccgcagcccagtccgagagaagctcggaattgaaatgcgcggctacggattactctccaacagtatcaccatctacttcaaggcggactgcacaaaatggatggctggacgcaacctcggcgtggtctatgtcaacaatcctgacgttcgcggcttcttccgcttgacgcgggaggccaagagtggcttcttgggtgtgaacaccgtaggagatgtcagccgcccggaggcgaacaatgtcgctgaaggtatcacggcagagcgctgcgtcgaaattgtacgttccgctgtgggcattcccgatctggaagttgaaattgagggcattgccccgtggcgcgccgttgccgatgtggctgaccgttacagaagtggaaatgtgtttctcatcggcgacgccgcacacgtcgttcctcctaccggcggattcggcgggaacactggtgttcaggacgcgcacaaccttggttggaagctggcttcagtactaaagggtcaagcagggccggctctgctcgacacctacgaggaggagcggcgcccggtcggtcaacttacgatcgagcaggcctattcccgatatgtgctgcggatcgcacctgaactgggccgtgaaacgatgaagcctgtggtcgacgacttgagcatggaaattggctaccgctatttctcatcagccatcttgtcgaacgaaaagcggggagatcgtgtttacgttgatccgcgcacgtcgttcagcttgcctgggaccagagtgggacacctcgtctttcagcgagacgggaaatcgatggcgaccctggacgtttgtgccggtggtatgacccttcttgccggagcgggcggcgtcgcctggtgtcagtcgacgactgaagctgccgtaaagctgggtatcgaggtcgagtcgaacgtcattggaaacgcgggtggactgacagatgtttcaggtcgcgcgctcgaggttctcggaatcgaaagcgcaggtgcaattctggtgcgccccgacggtttcgtggcttggcgctcagaacccggtgaggccgcgagtgtcgcgaggatgatcaacgtgctgaccgctgtaatgtgtctccagagtcagcgcgtagatgcatcggtcgtagctgcctaa

SEQ ID NO: 22 - DNA, 945 bp, Pseudomonas putida
atggccatcaccgacattgcctatttggtaaacgaccacaccgacctggaagcggcagagcgcttctacacggacttcggcttgaaggtcgcctaccgcaccgctgacgagatcggctttcgacccgctctggcgcgtggttactgctacgtggcacgcaaatcgagcaagcccggtctgcgcgcaattgccttcaccgccagcacgcgggccgatctggatgtcgcggcgcgtttccctgaggcatcgcccatcacgccaatcgagcgtgaaggtggtggcgataaagtgatgctccagagcccggacggcctgccgttcgagatcgtgcacggcatccattcctacgaggaactggcggtgcgccccgccttgacgttcaatacgggccgcgtcaaacgccgccacggagagttccaacgcccgccgctggagccagcgcccatcttgcggctggggcacgtcgccctgctgacgaacgacttcaagcgcaatttcgagtggatgcagtcgcgtctcggactacggccgaccgacaccatgtatggcaaggacaagaatgacctcgtcggctcattcatgcacctttccggcggtggcgagtggacagatcatcactcgcttgcccttttcccggacccgagtccccgcgtccaccacttctcgttcgaggtggaggacctcgatgcgcagggaatgggaaaccaatggctgatcaagcacgggtggaagcatacctggggcattggtcgtcacatatacggcagccagatcttcgattactggttcgatcccgacggcaacattgtcgagcatttcaccgatggcgaccttgtccgtcccggctgtaagccaggcttggtcgcgctcgacgagtcgctctttgcttggggtccgccgatgcagcccgccaacttcgtcgaactggctgcacaccacgaatga

SEQ ID NO: 23 - DNA, 1527 bp, Pseudomonas putida
atgtctgcagttcgagagtcgctcagtgatcaagtccccaccgtcaaactactgatcgacggcaagttcgtcgattccaatacgacggaatggctggatgacgttaatccggcgacgcaggccgttctgtcacgtgtccctatggcaaccgccgaagaggtgaacgccgccgttgcggcagccaagcgcgcattcgcaagctggaggcacgcaccaatcgggacaagggcgcgaattttcctcaagtaccagcaactgattcgggaaaacatgggggaactggccgcattgctgacggctgagcaaggcaagacccgattggatgccgaaggcgacatcttccgcggactcgaggtggttgaacacgctgcggccatcgggaacctgcaactaggcgaacttgccaacaacgttgccaagatggtggatacgtacacgcgtctgcagcccatcggtgtttgtgcgggcatcacgccattcaacttccctgcaatgattccgctgtggatgtttccgatggctatcgcatgcggcaacaccttcgtgctcaagccatccgagcaggatcccatggtgaccatgcgcctcgtcgaactggcgctcgaggccggcgtcccatccggtgttctgaacgtggtacatggcggagcgcaggtcgttgacgctatctgcgatcatcccgacatcaaggcgatctcgtttgtggggtccacccgtgtaggcacgcatgtctacaaccgggccacgctggcaggcaagcgtgtgcaatgcatgatgggcgccaagaatcacgcgatcctcatgcccgatgccaaccgggagcagtcgctgaatgcgatcgcgggcgccgctttcggcgctgctggccagcgttgcatggctctgccggtgctggtcatggttggcgaggcacaaaggtggctgcccgatttggtcgccaaggccaaggcactgaccgtcaatgccggcgatgcagcaggtgccgatgttggtccgcttatcagcccatccgcgtgtggccgagttcgtgaactaattggtgagggcgttgaggctggcgccaagctcgaactggatggccgtgctgtcagcgtctccgggtacgagaaaggcaacttcgtcggcccgaccatcttttctggcgtcaagccgggaatgtcaatctatgacatcgagatcttcggccccgtgctctgcgtgattgcggcggacaacctcgacgaggcgatcgagatcatcaatgccaatccgagcgggaacggcaccgccattttcacgcagtcgggtgcagcggcgcgcaggttcgaagaggatatcgacgttggacaggtaggcatcaatgtgccgatccccgtgcctgttccgatgttctcctttactggctctcgcgcctcgaagctgggcgatctcggtccttacggcaagcaggtcatcatgttctacacgcagaccaagaccatcaccgcacgctggttcgacgatgccagcgtccgcaagaacgtgaacacgaccatcgcgttggactga

SEQ ID NO: 24 - DNA, 846 bp, Pseudomonas putida
atgaaattggccagctttttgttggatgggaagcgtcgcagtggggccttgactgacaagggcctcgcagtgttcgatgtcgacggcgacatcggtgacttgctgcgcaaagggaccgacctcgcgggcttcgacgcattgctcgccaagagctcgcgcatcgtcgacgcggccagtgtgacctttgtgcaaccgtcggccgagccgcccaaaatcttctgtgtgggattgaactacgctgatcacacggccgagagtccgtacgaacagcccgactatccgaccatctttttgcgcgtggctaccgctacgacggggcacggcgcgagtattgccttgcctgatctgagcgagcaactcgactatgaaggcgagatgattgctgtgctgggcaagggtggcaagcgtatcccgaaggagcgtgcgcatgagcacgtgtttggctacgctgtcggcaacgaggtgtcggttcgcgactaccagttcaagtcgccgcagtggaccatcggcaagaacttcgacggcaccggagcatggggaccgtacatcgtcaccgccgacgaactcccgctggggggacatggtctgaagatcgaggttcgcctgaacggcaagacggttcagtcatcgaataccgggcacatgatctttgacgtcgccaccatcatctcgaccatcagccaggccatcactttgcagcccggtgacatcatcttcacgggaacgcctgctggtgtgggccttggccacaagcctccgctgtggatgaaggatggcgacaaggtcgaggtcgagatcgagggcatcggtctgctcaagaactcgatcgccaaagaggtcacttga

SEQ ID NO: 25 - DNA, 1000 bp, Escherichia coli
atggaaaactttaaacatctccctgaaccgttccgcattcgtgttattgagccagtaaaacgtaccactcgcgcttatcgtgaagaggcaattattaaatccggtatgaacccgttcctgctggatagcgaagatgtttttatcgatttactgaccgacagcggcaccggggcggtgacgcagagcatgcaggctgcgatgatgcgcggcgacgaagcctacagcggcagtcgtagctactatgcgttagccgagtcagtgaaaaatatcttcggttatcaatacaccattccgactcaccagggccgtggcgcagagcaaatctatattccggtactgattaaaaaacgcgagcaggaaaaaggcctggatcgcagcaaaatggtggcgttctctaactatttctttgataccacgcagggccatagccagatcaacggctgtaccgtgcgtaacgtctatatcaaagaagccttcgatacgggcgtgcgttacgactttaaaggcaactttgaccttgagggattagaacgcggtattgaagaagttggtccgaataacgtgccgtatatcgttgcaaccatcaccagtaactctgcaggtggtcagccggtttcactggcaaacttaaaagcgatgtacagcatcgcgaagaaatacgatattccggtggtaatggactccgcgcgctttgctgaaaacgcctatttcattaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtacaccgagtgcagaaccctttgcgtggtgcaggaaggcttcccgacatatggcggcctagaaggcggcgcgatggagcgtctggcggtaggtctgtatgacggcatgaatctcgactggctggcttatc